(19) United States
(12) Patent Application Publication
(10) Pub. No.: US 2023/0405054 A1
(43) Pub. Date: Dec. 21, 2023

(54) NOVEL NR1 ES-DERIVED NEURAL STEM CELLS HAVING A NORMAL KARYOTYPE AND USES THEREOF

(71) Applicant: The Board of Trustees of the Leland Stanford Junior University, Stanford, CA (US)

(72) Inventor: Gary K. Steinberg, Redwood City, CA (US)

(21) Appl. No.: 18/029,875

(22) PCT Filed: Oct. 11, 2021
(86) PCT No.: PCT/US2021/054362

§ 371 Date: Mar. 31, 2023

Related U.S. Application Data
(60) Provisional application No. 63/090,671, filed on Oct. 12, 2020.

(51) Int. Cl. A61K 35/30; A61P 25/00; A61P 9/10

(52) U.S. Cl. CPC A61K 35/30; A61P 9/10; A61P 25/00

(57) ABSTRACT

Compositions and methods are provided relating to NR1 ES-derived neural stem cells. The cells are useful in methods of treatment for an adverse neurologic conditions, including without limitation stoke.

### CROSS REFERENCE TO RELATED APPLICATION

The present application claims the benefit of and priority to U.S. Provisional Patent Application No. 63/090,671, filed Oct. 12, 2020, the entire disclosure of which is hereby.

### BACKGROUND

To date there have been no effective treatments for improving residual structural and functional deficits resulting from stroke. Stroke is the leading cause of adult disability, with the highest annual incidence of any neurological disorder, including Alzheimer's disease, traumatic brain injury, epilepsy and Parkinson's disease. Each year 795,000 people in the US will experience stroke, and approximately 7 million Americans are currently living with disability from stroke. Stroke-related costs are >$34 billion dollars/year and these numbers are predicted to rise as the age of the general population increases. Due to improved acute medical management, the stroke mortality rate is declining, thus changing stroke from an acute killer to a chronic disabling disease. Yet few treatment options exist to target this chronic phase of stroke. Such treatments for recovery remain focused on physical medicine-occupational therapy, physical therapy and speech/cognitive therapy. However, these are time consuming, costly, have a very modest benefit on functional recovery, and are not effective in further improving neurologic function more than 6 months following a stroke. These facts define a common, devastating, chronic disease with no existing medical therapy and establish stroke recovery as a major area of unmet medical need.

### SUMMARY

Compositions and methods are provided relating to NR1 ES-derived neural stem cells, which cells express trophic factors that are active in restoring neurologic function in a subject, where the trophic factors augment endogenous neural repair processes. The cells are not genetically manipulated or altered. The cells have a normal karyotype. Other benefits of the cells include improved viability after thaw and before transplant (12 hrs viability); efficacy at low doses, and a robust, scalable cGMP manufacturing process.

The disclosed NR-1 cells are useful in methods of treatment for adverse neurologic conditions, including without limitation stroke or traumatic brain injury. In some methods, the NR1 cells are delivered intracerebrally into a patient in need of treatment thereof. The NR-1 cells can be administered by implantation of an effective dose of the cells into or near the cerebral cortex of the patient, wherein the cerebral cortex of the subject may include any of the prefrontal cortex, motor association cortex, primary motor cortex or primary somatosensory cortex. Such administration may be, for example, cortical or sub-cortical, where a subcortical area of the brain may be any of the hippocampus, amygdala, extended amygdala, claustrum, basal ganglia, or basal forebrain.

In some embodiments, an effective dose of NR1 cells are delivered to a patient for treatment of stroke. In other embodiments, an effective dose of NR1 cells are delivered to a patient for the treatment of a neurologic condition selected from ischemic stroke, chronic hemorrhagic stroke, subacute ischemic and hemorrhagic stroke patients, patients with traumatic brain injury, spinal cord injury, Parkinson's Disease, ALS (Lou Gherig's Disease), and Alzheimer's Disease.

The NR1 cells are shown to secrete factors that improve neurologic function by enhancing multiple native (endogenous) molecular and cellular mechanisms, including modulation of the immune system, neovascularization and improving the excitatory/inhibitory balance of neural networks. The NR1 cells do not work through integration and cell replacement of neurons, astrocytes oligodendrocytes or other cells in the brain.

NR1 ES-derived neural stem cells can be characterized as expressing one or more of ColA1, LGALS1, TGF-B3, TIMP1, COL6A1, COL3A1, MMP2, SPARC, NRG3, SDF1(a), Galectin 1, FGF18, CSF3, CCL2 (aka MCP-1), FGF7, FGF17, PAI1/serpine 1, VEGF-A, MCP1 (CCL2), and/or SDF1α or a combination thereof. The expressed trophic factors can augment the repair processes in neural neovascularization by increasing blood flow and vascular signals within the cortical and/or subcortical brain area and increasing structural plasticity. Increased structural plasticity may involve, for example, synaptogenesis, dendritic sprouting, axonal sprouting, etc. For example, trophic factors may comprise a combination of a growth factor, cytokine and/or extracellular matrix protein. The extracellular matrix protein may be any of a ColA1, LGALS1, TGF-B3, TIMP1, COL6A1, COL3A1, MMP2, SPARC, NRG3, SDF1(a), Galectin 1, FGF18, CSF3, CCL2 (aka MCP-1), FGF7, FGF17, PAI1/serpine 1, VEGF-A, MCP1 (CCL2), and/or SDF1α or a combination thereof.

In some embodiments the effective dose of cells is at from about 1×10⁶, up to about 10⁸ cells per individual dose, and may be from about 2.5×10⁶ to about 2×10⁷, e.g. about 2.5×10⁶, about 5.0×10⁶, about 10×10⁶, about 20×10⁶/dose. The concentration of cells for delivery may be from about 10⁶ to about 10⁸ cells/ml of excipient, e.g. about 8.3×10⁶, about 16.6×10⁶, about 33.3×10⁶, about 66.6×10⁶/ml excipient.

In some embodiments, an isolated population of mammalian NR1 ES-derived neural stem cells having a normal karyotype that secrete trophic factors comprising a combination of a growth factor, cytokine and/or extracellular matrix protein is provided. In some embodiments, the NR1 cells are derived from a non-genetically modified NR1 ES neural stem cells having abnormal karyotype and cultured for after at least 15, 16, 17, 18 19, 20 or more passages so as to exhibit a normal karyotype.

In some embodiments a method is provided for production of human NR1 ES-derived neural stem cells having a normal karyotype under defined cell culture condition, wherein the method comprises: (a) culturing human NR1 ES-derived neural stem cells on a solid support in a cultured medium with glutamax, N2 supplement, B27 without RA, EGF, FGF, LIF and PHS in the absence of feeder cells; and thereby, producing human NR1 ES-derived neural stem cells in vitro under defined cell culture condition.

Administration of the NR1 ES-derived neural stem cells may involve implantation of the NR1 ES-derived neural stem cells into or near the cerebral cortex of the subject, wherein the cerebral cortex of the subject includes is any of the prefrontal cortex, motor association cortex, primary motor cortex or primary somatosensory cortex. The subcortical area of the brain may be any of the hippocampus, amygdala, extended amygdala, claustrum, basal ganglia, or basal forebrain. The cortical areas of the brain may be of the prefrontal cortex, motor association cortex, primary motor cortex or primary somatosensory cortex. Administration may be at least one week after the stroke or traumatic brain injury, and may be in an individual with hemiparesis from stable ischemic stroke that occurred from about 1 month, about 2 months, about 3 months, about 4 months, about 5 months, about 6 months and up to about 60 months previously.

Increased structural plasticity may involve any of synaptogenesis, dendritic branching and axonal sprouting. Restored neurologic function may partially or fully restores an upper extremity function, e.g. raising or lifting an arm of the subject. Restoring neurologic function may include restoring the ability to walk or balance or reversing a gait impairment, inability to walk, or loss of balance, e.g. wherein gait impairment comprises any of decreased walking velocity, asymmetric walking pattern, decreased stride length, increased stride width, prolonged swing phase of affected limb, diminished ability to negotiate physical obstacle, diminished ability to adjust walking to changes in terrain, loss of rhythmic movement, diminished ability to move across a beam and a combination thereof. Reversing the impaired mobility in the subject may comprise any of greater walking velocity, an increase in symmetric walking pattern, greater stride length, decreased stride width, reduced duration of swing phase of affected limb, greater ability to negotiate physical obstacle, greater ability to adjust walking to changes in terrain, increased rhythmic movement, greater ability to move across a beam or ladder and a combination thereof.

NR1 is the product from a progenitor cell line, originally derived from the H9 human Embryonic Stem Cell (hESC) line, by culture in neural-inducing conditions. It is a stable cell line that is not gene-modified. NR1 can be manufactured from the NR1 cGMP Master Cell Bank and exhibits consistent growth and quality attributes over a wide range of passages to p30 post-drug product. This evaluation gives confidence in the clinical drug product lots, which may be expanded only 3 passages from the MCB. Along with consistent growth characteristics, the attributes maintained across the 3 passages of clinical material production include normal karyology and levels of residual hESCs of less than 0.01%.

In some embodiments an individual is treated with one or more doses of NR1, stereotactically implanted into grey or white matter sites adjacent to subcortical infarct region. The one burr-hole craniotomy technique may be used, and cells implanted using needle tracks with deposits in each track at varying depths around the damaged area.

In some embodiments, the brain lesion may be located outside the cortex or subcortical brain region of the subject, e.g. wherein the brain lesion is located at or about the cortex or subcortical brain region of the subject.

A method for inhibiting, reducing or reversing a motor deficit in a subject after a stroke by increasing exogenous trophic factors in the cerebral cortex to augment endogenous neural repair processes in the subject comprising administering to the subject a therapeutically effective amount of neural stem cells, which express trophic factors, so as to restore neurologic function in the subject so as to inhibit, reduce or reverse the motor deficit of the subject.

In some embodiments a method is provided for treating a motor deficit of a subject after a stroke by increasing exogenous trophic factors in the cerebral cortex to augment endogenous neural repair processes in the subject comprising administering to the subject a therapeutically effective amount of neural stem cells, which express trophic factors, so as to restore neurologic function in the subject thereby treating the motor deficit of the subject.

In some embodiments a method is provided for a rehabilitating subject suffering from a motor deficit as a result of a stroke by reducing or reversing the motor deficits of the subject by inhibiting, reducing or reversing a motor deficit, e.g. as the result of a stroke.

### BRIEF DESCRIPTION OF THE DRAWINGS

FIG. 1. Teratoma formation as a function of H9 and NR-1 cell content.

FIG. 2. Flow chart for drug substance production.

FIG. 3. Flow chart for culture and pooling of NR-1 cells.

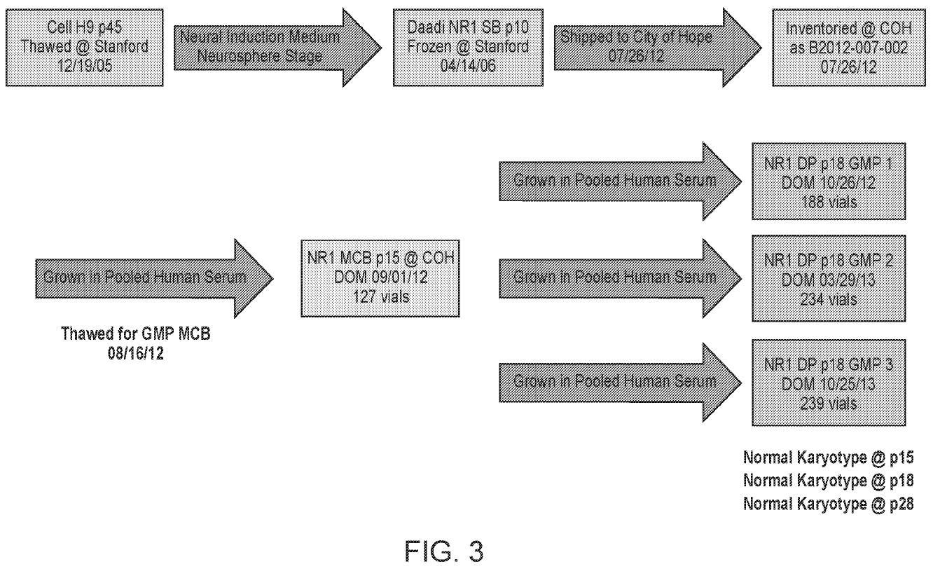

FIG. 4. Elevated body swing test of motor asymmetry.

FIG. 5. Mean neurologic score test of sensimotor function.

FIG. 6. Rotorod test of motor coordination.

FIG. 7. VP score over time comparing NR-1 doses.

FIG. 8. Vibrissae-Paw test comparing NR-1 doses.

FIG. 9. Posture-Reflex test comparing NR-1 doses.

FIG. 10. Modified Neurological score test comparing NR-1 doses.

FIG. 11. Histology slides comparing effects of NR-1 doses.

FIG. 12. Histology slides comparing effects of NR-1 doses.

FIG. 13. Comparison of L2 intensity and response curves.

FIG. 14. NR1 synaptogenesis values.

FIG. 15. GO biological process chart.

FIG. 16. Plots of monocytes and granulocytes post-transplantation.

FIG. 17. Expression of cytokines by M1 and M2 macrophages.

FIG. 18. Expression of cytokines by M1 and M2 macrophages.

FIG. 19. Protein expression of unstimulated macrophages.

FIG. 20. NR-1 survival time course.

FIG. 21. Evaluation of gene expression.

FIG. 22. Evaluation of gene expression.

FIG. 23. Microglia cells in the presence or absence of PLX.

FIG. 24. Microglia histology.

FIG. 25. Brain histology.

FIG. 26. Staining for Iba1 and GFAP.

FIG. 27. T2 MRI analysis.

### DETAILED DESCRIPTION

### Definitions

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease (e.g., reduction in viral titers).

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; avians, and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

### NR1 Cells

The NR1, from Neural Regenerative 1, cell line is a unique investigational cell therapy product developed to treat the motor sequelae of stroke and traumatic brain injury by direct intracerebral injection. Such motor deficits can be extremely disabling and any improvement in arm and/or leg motor control significantly improves subjects' health and quality of life. In animal stroke models NR1 has shown efficacy to improve motor functional control, and NR1 demonstrates an excellent safety profile.

NR1 is the drug product from a progenitor cell line, originally derived from the H9 human Embryonic Stem Cell (hESC) line, by culture, in neural-inducing conditions. It is a stable cell line that is not gene-modified. NR1 (Drug Product) is manufactured from the NR1 cGMP Master Cell Bank (NR1 MCB) and exhibits consistent growth and quality attributes over a wide range of passages to p30 post-drug product. Along with consistent growth characteristics, the attributes maintained across the 3 passages of clinical material production include normal karyology and levels of residual hESCs as assessed by qPCR for Oct and Rex1.

The drug, NR1, is a unique embryonic stem cell-derived product being developed for the treatment of chronic ischemic subcortical stroke with motor deficit. As the core of the stroke consists of nonviable tissue with no blood flow, the transplanted cells will not survive even transiently in this region. Therefore, the administration target for NR1 is the subcortical portion of the brain tissue adjacent to the subcortical stroke, a region shown in functional human brain imaging and nonclinical studies to be active in tissue repair and reorganization. This administration is consistent with nonclinical pharmacology studies in which injection coordinates were identified to directly deliver NR1 into tissue adjacent to the stroke lesion in rodent models of subcortical and cortical ischemic stroke, based on prior determination of the average size and location of lesions in these respective models. With this model, sensorimotor and other neurologic outcomes in animal models were positive and indicate that brain subcortical stroke tissue can be stimulated to at least partially restore neurologic function in subcortical and cortical regions. Because this same activity with restoration of neural circuits and clinical improvement can similarly occur in human brains after stroke, this study is proposed and supporting nonclinical studies designed as described below.

NR1 transplantation significantly increases motor circuit activity in the peri-infarct cortex after transplantation, while inhibitory connections are reduced in magnitude and delayed in onset. NR1 cells increase motor circuit activity by releasing individual neurons in the peri-infarct region from inhibitory control. NR1 cells can affect the plasticity of neuronal cells, e.g. by secreting factors that enhance synaptogenesis of retinal ganglion neuronal cells (RGCs). NR1 transplantation is linked to expression of genes associated with plasticity. NR1 cells cause a specific and differential expression of genes involved in stimulating adult neural precursor cell proliferation, neuronal differentiation, gated channels directly involved in the electrophysiological properties of neurons, axon guidance, axonogenosis, and synaptogenesis.

NR1 secretes proteins associated with brain repair, including extracellular matrix (ECM) remodeling (involved in most brain repair mechanisms) and processes related to inflammation and axon guidance. Specifically, NRG3 and TGFβ3 are upregulated in the stroke environment in vivo.

The immune response after transplantation by NR1 is shown to change macrophages from an M1/pro-inflammatory state to an M2/anti-inflammatory state. The T2-FLAIR MRI may be used as a clinical predictor of NR1-induced functional recovery in stroke subjects. The T2-MRI FLAIR signal is associated with inflammation. It can be used with a second non-invasive imaging modality, PET imaging of the translocator protein 18 kDa (TSPO), called TSPO-PET. TSPO comprises activated immune cells including brain resident immune cells, microglia and astrocytes, and infiltrating myeloid cells, such as monocytes/macrophages, neutrophils and dendritic cells. TSPO levels are low in the healthy brain but upregulated under inflammatory conditions. TSPO-PET radioligands thus serve as a useful index of neuroinflammation. These assays can show immunomodulation as a effect of NR1 facilitating stroke recovery.

NR1 cells can cause a specific and differential expression of genes involved in stimulating adult neural precursor cell proliferation, neuronal differentiation, gated channels directly involved in the electrophysiological properties of neurons, axon guidance, axonogenosis, and synaptogenesis. The cells contribute to the plasticity of peri-infarct regions after stroke by releasing individual neurons in the peri-infarct cortex from inhibitory control, as well as by modulating plasticity by secreting factors that stimulate synaptogenesis.

Some of the mostly highly expressed secreted genes by NR1 in vivo include: Col1A1 (collagen type 1 alpha 1 chain), which is involved in ECM organization and can affect leucocyte migration and inflammation4; LGALS1 (galectin 1), which has been shown to shift macrophage polarization to an M2-like (more anti-inflammatory) state in vivo after stroke5; TGFβ3, which regulates ECM formation and has both pro- and anti-inflammatory effects; TIMP1, an inhibitor of MMPs that is involved in ECM degradation. It also has MMP-independent actions, acting as a growth factor with a role in myelin repair. Genes involved in plasticity include: COL6A1, COL3A1, MMP2, SPARC, NRG3, and SDF1.

NR1 is formed from expandable and homogenous long-term cultures in vitro that are stable and infinite without changes in phenotype or loss of karyotype stability. The manufacturing process for NR1 does not require repeated use of hESCs and differentiation into neural progenitor cells, rather, vials of NR1 Master Cell Bank (MCB) cryopreserved at passage15 (p15) are thawed and expanded in defined culture media in feeder-free adherent culture to p18 to produce NR1 Drug Substance (DS). No vials of NR1 DS are produced, as such, because the manufacture of the DS is part of a continuous process including the fill, finish and cryopreservation as NR1 DP.

### NR1 Production

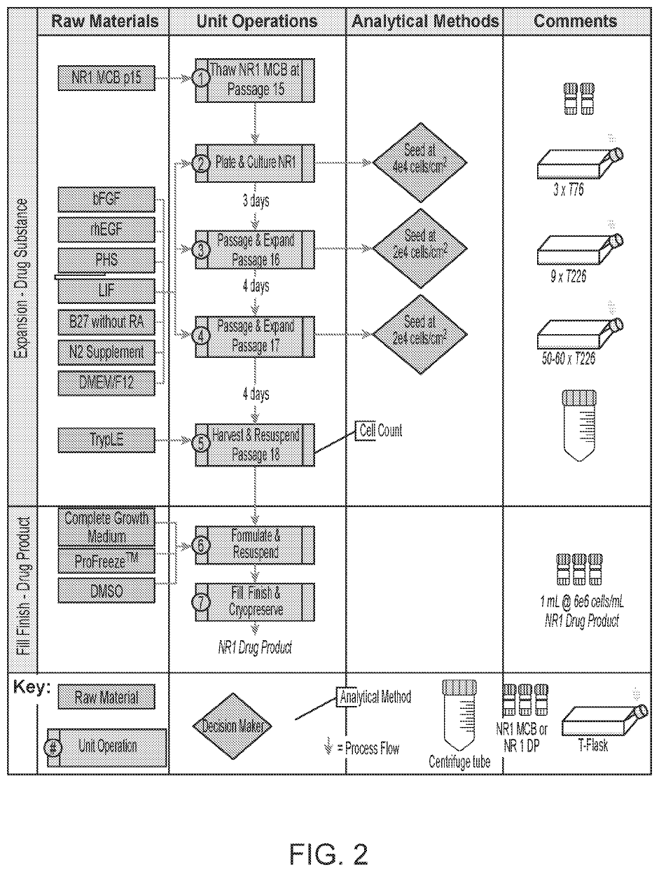

NR1 drug substance and drug product are manufactured as cGMP compliant, initiated with generation of the cGMP NR1 MCB. An exemplary “high level” process flow diagram of the NR1 manufacturing process is provided in FIG. 2. During cell expansion, culture passage numbers increase by one, after the monolayer is exposed to trypsin; (e.g. when passage 16 cells are seeded, then expanded into a near confluent monolayer and trypsinized, the resulting suspension is considered passage 17, and so forth). Bulk Drug Substance is identified in the process flow diagram as the harvest and first resuspension step in #5 of the “unit operations” column and is continuously processed on the same day to make NR1. The NR1 drug product consists of 6×10⁶ of a specified passage NR1 cells cryopreserved in 1 mL of the final cryopreservation formulation consisting of 7.5% DMSO, 42.5% ProFreeze, and 50% Complete Growth Medium, per vial.

NR1 lots may be tested for one or more of 1) sterility (USP), 2) bacteriostasis and fungistasis, 3) mycoplasma (USP), 4) mycoplasmastasis, 5) endotoxin, 6) in vitro adventitious viruses, 7) cell viability, 8) cell count (strength), 9) karyotype, 10) identity (CD73 expression by flow cytometry), 11) purity (β-Tubulin-Ill expression by flow cytometry), and 12) impurities (Tra-1-60 expression by flow cytometry, and OCT4 and REX1 by RT-qPCR) and used only if standards are met for these parameters.

### Endogenous Neural Repair Pathways and Potential Mechanisms

Administration of NR1 provides for long-term neurological recovery of both cortical and subcortical stroke, when administered in either the subacute or chronic timeframe. Reduction of neuro-inflammation and increased peri-infarct neovascularization are early, known mechanisms of action. In all in vivo studies conducted using NR1 to date, observations, data and results have shown no evident clinical signs of toxicity, no macroscopic or microscopic evidence of tumor or teratoma formation, and no test article-related adverse findings.

In some embodiments the administration target for NR1 is the subcortical portion of the brain tissue adjacent to the subcortical stroke, a region shown to be active in tissue repair after stroke and in reorganization, as observed in functional human brain imaging. This same region has been shown to be active in tissue repair and reorganization in studies also using the same site of administration. Injection coordinates are identified to directly deliver NR1 into tissue adjacent to the stroke lesion, based on prior determination of the average size and location of lesions in these respective models.

In some embodiments, efficacy is evaluated by clinical and radiologic responses as well as utility of efficacy measurement tools. Subjects may be compared to their own stable pre-transplant baseline. Established scales and the primary assessment of efficacy may be neurologic functional outcome on the Fugl Meyer (FM) motor score at 6 months post-transplant. Exploratory endpoint outcome measures will include NIHSS, Comfortable Gait Speed (CGS) test, mRS and Neuro Quality of Life (QOL), and total FM score at 6 months after treatment.

### Treatment of Chronic Stroke

To date there have been no effective treatments for improving residual structural and functional deficits resulting from stroke. Stroke is the leading cause of adult disability, with the highest annual incidence of any neurological disorder. Due to improved acute medical management, the stroke mortality rate is declining, thus changing stroke from an acute killer to a chronic disabling disease. Yet few treatment options exist to target this chronic phase of stroke. Such treatments for recovery remain focused on physical medicine—occupational therapy, physical therapy and speech/cognitive therapy. However, these are time consuming, costly, have a very modest benefit on functional recovery, and are not effective in further improving neurologic function more than 6 months following a stroke. These facts define a common, devastating, chronic disease with no existing medical therapy and establish stroke recovery as a major area of unmet medical need.

Transplantation of cells derived from cord blood, bone marrow or brain tissue (fetal and adult) secretes factors that enhance sensorimotor function in experimental models of stroke. However, the normal human-derived somatic stem cells used in these studies are not amenable for large-scale cell production. In studies in the Ischemic Subcortical Stroke (ISS) model, animals have shown that intracerebral transplantation with NR1 (neural stem cell drug product) secrete factors that enhance endogenous recovery of motor function, without significant toxicity. Yet, unlike those stem cells previously used, NR1 is amenable to large scale production.

### Intracerebral Transplantation

Patients receive an effective dose of NR1 cells, which may be provided as a single or as multiple doses, stereotactically implanted into grey or white matter sites adjacent to a subcortical infarct region. An effective dose may be a fixed dose ranging from about 2.5×10⁶ cells, 5.0×10⁶ cells, 10×10⁶ cells, 20×10⁶ cells, 50×10⁶ cells, 100×10⁶ cells.

In some embodiments, a one burr-hole craniotomy technique is used, and cells implanted using multiple needle tracks with from 1, 2, 3, 4, 5, etc. deposits in each track at varying depths around the damaged area. In some embodiments the cells are manually administered using a delivery system comprising a stabilizing cannula with stylet and injection needle.

Due to the allogeneic nature of NR1 and its attendant preparation, recipients may be treated with an immunosuppressive regimen, including without limitation tacrolimus, standard-dose cyclosporine A (CsA), low-dose CsA, etc.

### EXPERIMENTAL

### Example 1

### Example 1

The Stanford University Neural Transplant Program (SUNTP) has developed a new cell drug product, NR1, from their NR1 (Neural Regenerative 1) cell line. NR1 is a unique investigational cell therapy product developed to treat the motor sequelae of stroke by direct intracerebral injection. Such motor deficits can be extremely disabling and any improvement in arm and/or leg motor control would significantly improve subjects' health and quality of life. In animal stroke models NR1 has shown efficacy to improve motor functional control, and NR1 demonstrates an excellent safety profile.

NR1 is the drug product from a progenitor cell line, originally derived from the H9 human Embryonic Stem Cell (hESC) line, by culture, in neural-inducing conditions. It is a stable cell line that is not gene-modified. NR1 (Drug Product) is manufactured from the NR1 cGMP Master Cell Bank (NR1 MCB) and exhibits consistent growth and quality attributes over a wide range of passages to p30 post-drug product. This evaluation gives much confidence in the clinical drug product lots which are only expanded 3 passages from the MCB. Along with consistent growth characteristics, the attributes maintained across the 3 passages of clinical material production include normal karyology and levels of residual hESCs as assessed by qPCR for Oct4 (below the assay limit of detection of 0.01%) and Rex1 (not detectable).

As noted above, NR1 cells are a human neural stem cell line, derived from the WiCell Research Institute WA09 (H9) human Embryonic Stem Cell (hESC) line and are the active component in the drug product (DP), NR1. NR1 is formed from expandable and homogenous long-term cultures in vitro that are stable and infinite without changes in phenotype or loss of karyotype stability. The manufacturing process for NR1 does not require repeated use of hESCs and differentiation into neural progenitor cells, rather, vials of NR1 Master Cell Bank (MCB) cryopreserved at passage 15 (p15) are thawed and expanded in defined culture media in feeder-free adherent culture to p18 to produce NR1 Drug Substance (DS). No vials of NR1 DS are produced, as such, because the manufacture of the DS is part of a continuous process including the fill, finish and cryopreservation as NR1 DP.

### Clinical Evaluation of NR1

A combined Phase 1/Phase 2 clinical study has been designed to assess the safety, tolerability, and obtain an indication of efficacy of intra-cerebral transplantation (ICT) of NR1 in subjects with chronic ischemic subcortical stroke (cISS). It is an open-label safety and tolerability study using stereotactic, intracranial injection of NR1 cells in subjects 18 to 75 years with hemiparesis from stable ischemic stroke. Stability is evaluated by identifying subjects who have remained stable during the screening and baseline period prior to surgery (at least one week apart). While primarily a safety study, efficacy parameters will also be evaluated.

Each of 4 cohorts receive single doses of NR1, escalating by cohort, which are stereotactically implanted into grey or white matter sites adjacent to the subcortical infarct region. The one burr-hole craniotomy technique is used, and cells implanted using 3 needle tracks with 5 deposits in each track at varying depths around the damaged area. Cell implantation is standardized for volume (20 μL/deposit) and rate of infusion (10 μL/min), with spacing between each implant of approximately 5-6 mm. Each deposit is expected to take approximately 2-3 minutes, with each needle track being completed within 15 minutes.

NRI is manually administered using a delivery system consisting of a custom manufactured stabilizing cannula with stylet and injection needle, manufactured by Bausch & Lomb, and a Hamilton Gastight 1700 series 100 μL borosilicate glass syringe. Currently there are no needles or syringes with FDA market clearance compatible with the administered dose volumes required for NR1 administration. To eliminate dead space, minimize damage to tissue made by the needle cannula and maximize dose accuracy, the NR1 injection needle and stabilizing cannula with stylet are custom manufactured.

The NR1 delivery system proposed for use is identical to that utilized in six prior (and one ongoing) clinical trials of stem cell therapeutics for stroke. It has been designed to provide and demonstrated to ensure consistent and accurate delivery of the stem cell dose in animals and in humans. In this study, NRI will be manually administered using a delivery system consisting of a stabilizing implant cannula and an injection needle attached to a borosilicate glass 100 μL syringe. The NR1 delivery system specifications have been designed to fit with the Leksell Stereotactic System device. In addition, this custom-designed injection needle and stabilizing cannula configuration has been used in prior Phase 1 and 2 clinical trials.

Due to the allogeneic nature of NR1 and its attendant preparation, all subjects will receive pretreatment and concomitant administration of oral tacrolimus (Prograf) to minimize any potential for acute host rejection reaction. In a published study of multiple immunosuppressant agents, tacrolimus was found to be more effective than both standard-dose cyclosporine A (CsA) and low-dose CsA. The selection of immunotherapeutic regimen is covered in a Clinical Protocol appendix describing the rationale and use of tacrolimus in the clinic. Low-dose tacrolimus was chosen for immune suppression in this study, and the dose and duration will adhere to the Stanford University Clinical Protocol for clinical tacrolimus use.

All subjects enrolled would receive the best medical practices and supportive care. The 4 doses expected to be tested are based on doses evaluated nonclinically, and are 2.5, 5.0, 10 and 20×10⁶ cells in a fixed volume (300 μL) with safety confirmed in each of the cohorts over the month after the last subject is treated prior to moving to the next higher dose cohort. In addition to assessment of both safety and tolerability, multiple neurological and functional parameters will be evaluated in an attempt to define which of the safe and well-tolerated doses will best facilitate clinical improvement in the direction of return toward normal function.

### NR1 Production

NR1 drug substance and drug product were manufactured cGMP compliant at the Center for Applied Technology Development (CATD), City of Hope (COH) GMP manufacturing facility. Production was initiated with generation of the cGMP NR1 MCB, which was vialed and cryopreserved on Sep. 1, 2012. From this “source” material, thus far three cGMP runs have been performed to manufacture the NR1 DS and NR1 DP. A “high level” process flow diagram of the NR1 manufacturing process from NR1 MCB (p15) is shown in FIG. 2. During cell expansion, culture passage numbers increase by one, after the monolayer is exposed to trypsin; (e.g. when passage 16 cells are seeded, then expanded into a near confluent monolayer and trypsinized, the resulting suspension is considered passage 17, and so forth). Bulk Drug Substance is identified in the process flow diagram as the harvest and first resuspension step in #5 of the “unit operations” column and is continuously processed on the same day to make NR1. The NR1 drug product consists of 6×10⁶ passage 18 (p18) NR1 cells cryopreserved in 1 mL of the final cryopreservation formulation consisting of 7.5% DMSO, 42.5% ProFreeze, and 50% Complete Growth Medium, per vial. It is important to emphasize that the NR1 drug product manufactured for clinical use and used in pivotal nonclinical studies was manufactured following cGMP compliance at the CATD, City of Hope (COH) GMP manufacturing facility.

All lots of NR1 drug product intended for use in definitive nonclinical and clinical studies have been and will continue to be manufactured under cGMP compliant conditions. The NR1 drug product was fully characterized and released for use after applying release criteria appropriate for the current early stage of clinical development. The three cGMP NR1 lots proposed for use in the Clinical Study have been tested for 1) sterility (USP), 2) bacteriostasis and fungistasis, 3) mycoplasma (USP), 4) mycoplasmastasis, 5) endotoxin, 6) in vitro adventitious viruses, 7) cell viability, 8) cell count (strength), 9) karyotype, 10) identity (CD73 expression by flow cytometry), 11) purity (β-Tubulin-Ill expression by flow cytometry), and 12) impurities (Tra-1-60 expression by flow cytometry, and OCT4 and REX1 by RT-qPCR). Tests for mycoplasma (USP), mycoplasmastasis, in vitro adventitious viruses, and karyotype were conducted with the NR1 DS. Specifications have been established to ensure safe and consistent manufacture of the NR1 drug product.

Pilot and GLP nonclinical experimental study data are presented based on the preponderance of the nonclinical study toxicology results, are consistent across nonclinical studies with NR1.

### Endogenous Neural Repair Pathways and Potential Mechanisms

Administration of NR1 results in long-term neurological recovery in standard animal models of both cortical and subcortical stroke, when administered in either the subacute or chronic timeframe. Data from these models suggest that reduction of neuro-inflammation and increased peri-infarct neovascularization are early, known mechanisms of action.

NR1 has an excellent safety profile, which has been supported by investigations in multiple stroke models, in the naïve condition, in neonatal and adult animals, and in genetically immunodeficient and pharmacologically immunosuppressed animals. In this wide range of animal models covering many possible immunological, age and health compromised circumstances, combined with the use of cell formulations to optimize cell product persistence, NR1 shows modified persistence relative to the long-term duration of its efficacy. In in vivo studies, the persistence, ongoing efficacy and mechanistic data have been correlated, in multiple instances, on an animal-by-animal and group average basis. In all in vivo studies conducted using NR1 to date (MPI, USF, Stanford), observations, data and results have shown no evident clinical signs of toxicity, no macroscopic or microscopic evidence of tumor or teratoma formation, and no test article-related adverse findings.

Potential tests for functional recovery were investigated, as well as several histology-based quantification endpoints, including host brain lesion size and host brain repair pathways, such as neovascularization, inflammation, and increased axonal sprouting by host neurons, referred to as plasticity. These endogenous repair pathways were previously investigated as surrogate measures of recovery and potential human neural progenitor cell (hNPC) in vivo mechanisms of action. In addition, the survival of transplanted cells in the brain was assessed to characterize NR1 persistence and its potential for post-transplant differentiation, migration, ectopic tissue formation and tumorigenicity.

### Proposed Clinical Evaluation of NR1: Summary

The primary objective of the proposed clinical study is to evaluate the safety and tolerability of escalating doses of NR1 cells, using increasing numbers of cells in a fixed volume, when administered intracerebrally at a single time point after injury to subjects with chronic ISS with or without cortical stroke. The study will be a dose escalation using up to 4 cohorts, with each cohort at a single dose level, of intracerebral administration of NR1 cells. Additional objectives are to evaluate clinical and radiologic responses as well as utility of established efficacy measurement tools. Subjects are compared to their own stable pre-transplant baseline. Further exploratory endpoints include monitoring changes in Magnetic Resonance (MR) Diffusion Tensor Imaging (DTI), Fluid Attenuation Inversion Recovery (FLAIR), and Resting State functional Magnetic Resonance Imaging (fMRI) also in comparison to baseline.

The primary objective is to evaluate the safety and tolerability of escalating doses of NR1 (increasing numbers of cells in a fixed volume) administered intracerebrally at a single time point, after injury, to subjects with chronic Ischemic Subcortical Stroke (ISS) with or without cortical stroke. The study is conducted in adult subjects (18 to 75 year of age) with hemiparesis from stable ischemic stroke that occurred 6-60 months previously as over 90% of ischemic stroke subjects are stable by 90 days post stroke.

The proposed study uses a dose escalation design for intracerebral administration of NR1 cells. The administration target for NR1 is the subcortical portion of the brain tissue adjacent to the subcortical stroke, a region shown to be active in tissue repair after stroke and in reorganization, as observed in functional human brain imaging. This same region has been shown to be active in tissue repair and reorganization in nonclinical studies also using the same site of administration. The clinical administration is consistent with nonclinical pharmacology studies in which injection coordinates were identified to directly deliver NR1 into tissue adjacent to the stroke lesion in rodent models of subcortical and cortical ischemic stroke, based on prior determination of the average size and location of lesions in these respective models. The demonstrated safety, tolerability and efficacy in nonclinical studies at exaggerated doses, allowing for differences in brain size, is the basis for the clinical study proposed.

Secondary objectives include evaluating attendant clinical and radiologic responses as well as utility of efficacy measurement tools. Subjects will be compared to their own stable pre-transplant baseline. Subjects will be evaluated for safety and tolerability of NR1 post-operatively at 1-day, 1-week, and at 1-, 2-, 3-, and 6-months during the study. Safety and tolerability will be assessed by monitoring clinical symptoms, edema, inflammation, other pathological changes and clinical laboratory findings. At the same time points, efficacy data is collected using established scales and the primary assessment of efficacy will be neurologic functional outcome on the Fugl Meyer (FM) motor score at 6 months post-transplant. Exploratory endpoint outcome measures will include NIHSS, Comfortable Gait Speed (CGS) test, mRS and Neuro Quality of Life (QOL), and total FM score at 6 months after treatment.

Four successive cohorts receive escalating single doses of NR1, which are to be stereotactically implanted into grey or white matter sites adjacent to the subcortical infarct region. One burr-hole craniotomy will be created, and cells will be implanted using 3 needle tracks with 5 deposits for each track at varying depths around the damaged area. Cell implantation will be standardized as to volume (20 μL/deposit) and rate (10 μL/min), with spacing between each implant of approximately 5-6 mm. Each deposit is expected to take approximately 3 minutes, with each needle track being completed within 20 minutes. This surgical procedure may be modified as surgically appropriate if indicated. Both FDA and IRB will be apprised of need for changes as soon as such need is determined.

Subject cohorts are treated with increasing doses of NR1 using a traditional 3+3 trial design with each subject receiving only a single dose. The first 3 subjects will receive NR1 at a dose shown to be safe in animals based on extrapolation from animal toxicology studies. Subsequent cohorts are treated with an a priori determined increase in NR1 dose using a fixed volume. If subjects in a cohort do not experience a dose-limiting toxicity (DLT) defined using the CTCAE scale, another 3 subjects are started on treatment at the next higher dose level cohort following a 4-week observation hold between cohorts. If only 1 subject of the first three in a cohort exhibits a DLT, an additional 3 subjects will be treated at this dose level and, if no additional DLT occurs at this dose level, the total treated in the cohort is 6 subjects.

A DLT is defined as a Grade 3 or 4 clinical event on the CTCAE scale, version 5 that is determined by the investigator to be possibly related to NR1. Dose escalation will continue until at least 2 subjects in a cohort of up to 6 subjects exhibit DLT (i.e. >33% of subjects at that dose). The Maximum Tolerated Dose (MTD) is designated as either the dose level immediately below the dose at which 2 subjects have experienced a DLT or if the maximum dose is reached with no dose limiting toxicity, and additional 6 subjects will be treated at that dose to establish the MTD.

Safety monitoring will be by clinical symptoms, MRI (edema, inflammation, other pathological changes) and clinical laboratory findings. Subjects will then be followed for 15 years after NR1 implantation.

### Details of the NR1 Clinical Study

The drug, NR1, is a unique embryonic stem cell-derived product being developed for the treatment of chronic ischemic subcortical stroke with motor deficit. As the core of the stroke consists of nonviable tissue with no blood flow, the transplanted cells will not survive even transiently in this region. Therefore, the administration target for NR1 is the subcortical portion of the brain tissue adjacent to the subcortical stroke, a region shown in functional human brain imaging and nonclinical studies to be active in tissue repair and reorganization. This administration is consistent with nonclinical pharmacology studies in which injection coordinates were identified to directly deliver NR1 into tissue adjacent to the stroke lesion in rodent models of subcortical and cortical ischemic stroke, based on prior determination of the average size and location of lesions in these respective models. With this model, sensorimotor and other neurologic outcomes in animal models were positive and indicate that brain subcortical stroke tissue can be stimulated to at least partially restore neurologic function in subcortical and cortical regions. Because this same activity with restoration of neural circuits and clinical improvement can similarly occur in human brains after stroke, this study is proposed and supporting nonclinical studies designed as described below.

The primary objective evaluates the safety and tolerability of up to 4 escalating dose levels of NR1 (increasing numbers of cells in a fixed 300 μL volume) administered intracerebrally at a single time point after injury to subjects with chronic ISS with or without cortical stroke. Total study duration is expected to be 2.5 years, allowing 2 years for study enrollment.

The administration target for NR1 is the subcortical portion of the brain tissue adjacent to the subcortical stroke, a region shown to be active in tissue repair and reorganization in functional human brain imaging and nonclinical studies. This administration is consistent with nonclinical pharmacology studies in which rodent models of subcortical and cortical ischemic stroke demonstrated improved function following NR1 directly injected into tissue adjacent to ischemic stroke lesions.

Subjects are evaluated for safety postoperatively at 1-week, and at 1-, 2-, 3-, and 6-months during the study. During study safety follow up, at 12- and 24-months, indications of efficacy will continue to be followed. Also, during the study, subject safety monitoring will assess clinical symptoms, Magnetic Resonance Imaging (MRI) for edema and other pathological changes and clinical laboratory findings. Primary assessment of efficacy is neurologic functional outcome on the Fugl Meyer (FM) motor score at 6 months post-transplant, and secondary outcome measures will include NIHSS, Comfortable Gait Speed (CGS) test, mRS, Neuro Quality of Life (QOL) and FM motor score at additional time points.

Nonclinical studies were designed and conducted to support the safety, activity and evaluate risk of the administration at the proposed NR1 doses to be tested, 2.5, 5.0, 10 and 20×10⁶ cells in a fixed volume (300 μL) in the clinic. As designed for safe evaluation in the clinical study, safety and tolerability parameters will be evaluated and safety will be confirmed in each of the cohorts prior to moving to the next higher dose cohort. At least 11 and no more than 30 subjects will be treated. Subjects will receive single ascending doses of NR1 in a standard 3+3 study design. Cohorts will normally consist of 3 subjects, with an additional 3 subjects added to the cohort if a dose-limiting toxicity (DLT) occurs in one subject. As described previously, dose escalation will continue unless 2 subjects of the first 3 to 6 subjects in any one cohort experience a dose-limiting toxicity (DLT 33%). The Maximum Tolerated Dose (MTD) will be designated as either the dose level immediately below the dose at which DLT is reached, or the maximum dose reached in the highest dose cohort, cohort 4, if no dose gives rise to DLT. An additional 6 subjects will receive the MTD.

Subjects are required to discontinue antithrombotic therapy in the peri-operative period consistent with routine care practices and will be given tacrolimus to minimize the potential for acute rejection of the NR1 administered. Subjects enrolled will receive the best medical practices and supportive care.

NR1, as cryopreserved NR1 cells, are supplied to clinical sites in single-use vials, each containing 6×10⁶ cells, stored in vapor phase liquid nitrogen. “On Site Preparation” NR1 cells will be thawed, washed, re-suspended in a delivery solution comprised of PlasmaLyte-A™ buffer and Human Serum Albumin (HSA), then checked for viability and absence of organisms detectable by gram stain (immediately) and by 14 day USP sterility (retrospectively), and finally loaded into the cryovials for transport to the surgical suite. NR1 cells will be administered within 6 hours following completion of dose preparation.

NR1 cells, prepared as planned for clinical evaluation, have demonstrated no discernable safety concerns in nonclinical studies designed to detect toxicity, were it to occur. The NR1, so prepared, remains at the injection site without migration and NR1 has not shown any tumor production over 350+ animals treated with NR1. Of course, theoretical risks do exist, including generalized toxicity, tumorigenicity/teratoma formation, transmission of undetected human or murine viruses, or an immune response to transplantation of cells. All attempts to study the actual potential and minimize the NR1 cell adverse effects have been made and include: demonstration that no detectable residual H9 cells exists using Oct4 and Rex1 qPCR assays with a sensitivity of 0.01%. Additionally, an extensive study of possible adventitious agents has been conducted and levels were found to be below levels of detection.

Use of immunosuppression to avoid NR1 cell death has been standard in 2 nonclinical studies and is proposed for use in the clinical study. The risks due to tacrolimus will be reduced to the extent possible by maintaining a low whole blood trough level until Day 30, by tapering tacrolimus from Day 41 to Day 60 and by discontinuing tacrolimus at Day 60 after NR1 injection. In addition, subjects will be carefully monitored for potential tacrolimus toxicity by frequent laboratory tests for safety.

NR1 cells require intracerebral injection during an open surgical procedure following initiation of monitored anesthesia care (MAC), without general anesthesia. The surgery requires MAC, injection of local anesthetic, stereotactic-frame placement, burr-hole placement and opening of the dura. The risks of this procedure will be mitigated by using best surgical practices. The delivery model and surgical approach has been successfully used in clinical trials for other stem cell biologic agents.

### Nonclinical Evaluation of NR1

NR1 is a human embryonic stem cell-derived product being developed for the treatment of chronic ischemic subcortical stroke with motor deficit. The administration target for NR1 is the subcortical portion of the brain tissue adjacent to the subcortical stroke, a region shown to be active in tissue repair and reorganization in functional human brain imaging and nonclinical studies.

Pharmacology studies have been conducted to evaluate the mechanisms of activity in the animals, accompanied by studies to select the optimal animal model for toxicity/tumorigenicity/teratoma evaluation. The NR1 administration in animal models is consistent with the clinical administration route, in which injection coordinates are identified to directly deliver NR1 into tissue adjacent to the stroke lesion in rodent models of subcortical and cortical ischemic stroke, based on prior determination of the average size and location of lesions in these respective models. The animals so treated showed improved function.

### Evaluation of Activity

Pharmacology, biodistribution and toxicology studies were conducted with NR1 drug product to assess its efficacy, its persistence and migration in vivo, and its potential to cause adverse events, including the development of teratomas. Selected models of immunodeficient or immunosuppressed animals were used in all the studies to minimize the potential for rejection of the human NR1 cells.

The objective of two pharmacology studies was to measure the efficacy of NR1 transplanted by intracerebral (IC) injection. The primary pharmacology study was done in a model of ischemic subcortical stroke (ISS) in immunosuppressed Sprague-Dawley (SD) rats; this model is a well-characterized ISS model that is routinely used in the stroke field and provides the closest match to the proposed target indication of subjects with chronic ISS. A supportive pharmacology study used a model of ischemic cortical stroke (ICS) in immunodeficient athymic nude rats. This model is also widely used in the stroke field and it allows for assessment of NR1 efficacy specific to a cortical lesion.

### Evaluation of Activity and Mechanism of Action

Pharmacology studies were performed to test for improvement in functional studies and behavioral recovery of NR1 treatment in nonclinical rat models o ischemic subcortical stroke (ISS) and ischemic cortical stroke (ICS), Partial functional recovery was measured using a battery of motor and behavioral tests to quantify post-injury recovery of sensorimotor function. Histological evaluations were used to explore candidate mechanisms of action for potential correlation with NR1-induced changes in both studies. The pivotal efficacy study evaluated the potential therapeutic value of IC transplantation of NR1 in a rat model of ISS using immunosuppressed SD rats in the middle cerebral artery occlusion model of ISS. Immunosuppression was included to decrease the rejection of the human NR1 cells in this rat model. The system is a well-characterized ISS model that is routinely used in the stroke field and provides the closest match to the proposed target indication of subjects with chronic ISS.

The primary endpoint was to test the hypothesis that NR1 treatment at either 7 days or 28 days post-stroke would result in a statistical improvement in the elevated body swing test (EBST), with secondary endpoints of improvements in the neurological exam and the Rotorod test. The studies demonstrated that transplantation of the NR1 drug product after stroke has the potential to bring about at least a partial recovery of sensorimotor function as measured by both motor and neurobehavioral endpoints in animal models, including an ISS model of chronic stroke that most closely matches the proposed NR1 target indication of subjects with chronic ISS. A supportive study in a model of ICS provided data, with improved sensorimotor function measured using several different endpoints (the Vibrissae-Paw and Posture Recovery tests and an overall neurological assessment. Neurologic recovery was sustained for at least 3 months (total duration of the study) after transplantation of NR1 drug product. Histological assessment at 3 months after treatment suggested a link between increased peri-infarct blood vessel density and NR1-associated enhancement of functional recovery when NR1 was administered during the subacute phase (7 days) post-stroke and between decreased peri-infarct microglial/immune cell activation and NR1-associated enhancement of functional recovery in the high-dose NR1 in the chronic phase (28 days) post-stroke.

In the Stanford-5 study, the primary endpoint in this ICS model was to compare the functional recovery of animals treated with test vehicle alone (Plasmalyte-A plus 0.5% human serum albumin) to animals treated with test vehicle containing NR1 (4×10⁵ or 1×10⁵ cells/rat). A sensorimotor behavior test known as the Vibrissae-Paw Test was selected as the primary measure of functional recovery, since this test has reproducibly shown increased functional recovery following cortical ischemic stroke and hNPC treatment. Secondary endpoints included the Posture-Reflex Test, which has been found to positively correlate with the Vibrissae-Paw Test and is an assessment of sustainability of functional recovery. Tertiary endpoints included comparisons between the two active treatments for a trend in dose effect using the Modified Neurological Score Test. Recovery was partial but durable, with evidence of recovery sustained for as long as 2 months (the total duration of study) after NR1 treatment. Histology-based quantification endpoints, including host brain lesion size and host brain repair pathways, such as neovascularization, inflammation, and increased axonal sprouting by host neurons, referred to as plasticity. These endogenous repair pathways were previously investigated as surrogate measures of recovery and potential hNPC in vivo mechanisms of action. In addition, the survival of transplanted cells in the brain was assessed to characterize NR1 persistence and its potential for post-transplant differentiation, migration, ectopic tissue formation and tumorigenicity.

In both USF-1 and Stanford-5 recovery was partial but durable, with evidence of recovery as long as 2 to 3 months after NR1 treatment. The exact mechanism by which NR1 cells mediate their efficacious effect is not yet fully understood, but appears to be related, at least in part, to immunomodulatory function as reflected in treatment-related decreases in immune cell activation that correlate with the observed partial functional recovery after NR1 treatment, as well as possible effects on neural plasticity. Evidence suggests that NR1 has this effect due to the stimulation of processes believed central to stroke recovery, including the process of inflammation and the organization of the extracellular matrix, via in part due to the increased expression of genes known to shift macrophage polarization to a more beneficial M2-like (more anti-inflammatory) state in vivo. Furthermore, NR1 appears to improve the excitatory/inhibitory balance within the post stroke cortex. NR1 cell persistence data also suggest that the cells may survive as long as 6 months after transplantation into the brain, potentially enhancing the observed recovery.

As in USF-1, recovery in the Stanford-5 study was partial but durable, with evidence of recovery as long as 2 months (total duration of study) after NR1 treatment. The exact mechanism by which NR1 cells mediate their efficacious effect is not yet fully understood, but it appears to be related, at least in part, to immunomodulatory function as reflected in treatment-related decreases in immune cell activation that correlate with the observed partial functional recovery after NR1 treatment, as well as possible effects on neural plasticity. Evidence suggests that NR1 has this effect due to the stimulation of processes believed central to stroke recovery, including the process of inflammation and the organization of the extracellular matrix, via in part due to the increased expression of genes known to shift macrophage polarization to a more beneficial M2-like (more anti-inflammatory) state in vivo. Furthermore, NR1 appears to improve the excitatory/inhibitory balance within the post-stroke cortex. NR1 cell persistence data also suggest that the cells may survive as long as 6 months after transplantation into the brain, potentially enhancing the observed recovery.

### Discussion of Selection of Animal Models for Biodistribution and Toxicology Testing

Persistence of NR1 cells in the selected animal models is critical to obtaining accurate measures of efficacy, distribution and toxicity. Several different approaches were evaluated to select the animal model with the best cell persistence. NR1 survival was compared in different immunodeficient rodent models, including adult and neonatal athymic nude rats and adult NSG mice. The highest rate of cell survival and persistence was measured in immunodeficient adult athymic nude rats, which were used to further test approaches to improve persistence by depletion of specific immune cell populations. Neither depletion of microglial cells, which act as macrophages in the CNS, nor depletion of Natural Killer cell numbers increased the survival of NR1 in the adult athymic nude rat. NR1 cell persistence was increased by including Matrigel in the cell injection. Matrigel is a heterogeneous mixture of structural proteins and cellular growth factors known to improve the survival of stem cells. In the pilot studies, inclusion of Matrigel significantly improved long-term NR1 cell survival. Matrigel was not used in the pharmacology and distribution studies because of possible confounding effects, though it was included in the injection medium only in the toxicology studies to improve NR1 survival and growth.

### Discussion of GLP Biodistribution Study and NR1 Persistence

The GLP biodistribution study was conducted in the ISS model in immunosuppressed Sprague-Dawley rats to provide the most complete assessment of the persistence and migration of IC-injected NR1 cells in a stroked animal that best models the clinical condition of human subjects with chronic stroke. At time points ranging from 24 hours to 60 days post-transplantation slides were prepared from the brain and selected peripheral tissues for analysis of the presence of NR1 cells by IHC and frozen samples were collected for qPCR analysis. IHC showed the occurrence of NR1 at minimal to mild to moderate magnitude of NR1 cells in brains of animals in both the acute and chronic stroke groups as long as 45 days after transplant with a general decreasing trend in both incidence and magnitude. The presence of NR1 cells was limited to the hemisphere of the brain into which the cells were injected, and cells were not detected in other organs examined in any animal. The qPCR analysis showed the presence of NR1 cells at the injection site in animals as long as 1-week post-treatment. No NR1 cells were detected in any area of the brain distant from the injection site or in peripheral tissues using either IHC or qPCR, with the possible exception of cells inadvertently injected into a brain ventricle in a few animals and findings related to contamination at necropsy or via the tissue processing procedures.

Cell persistence studies were also conducted in the GLP toxicology study, but in the presence of Matrigel. Using both IHC and qPCR analysis NR1 cells were detected at minimal to moderate levels in most treated animals as long as 180 days after treatment. As in the biodistribution study, NR1 cells were present at the injection sites, visualized as cavitated spaces microscopically, within the brain parenchyma or less frequently within the ventricular spaces, with no evidence of migration of NR1 cells away from the injection sites. The presence of NR1 cells in the ventricles was attributed to inadvertent injection of the cells into the ventricle during transplantation.

Overall, the NR1 biodistribution studies clearly showed no migration of NR1 cells away from the site of the intracerebral injection.

### Discussion of GLP Toxicology/Tumorigenicity/Teratoma Formation Study

As noted above, the selection of the athymic nude rat model for the six-month GLP toxicology study was based on extensive evaluation of immunocompromised rodent models and persistence of NR1 at the site of action. Studies evaluating NR1 persistence in the brain include three pilot toxicology studies in naïve (non-stroked) NSG mice or nude rats as well as a small persistence study conducted in naïve nude rats. No NR1 cells were detected in brain at 10 weeks post-transplant in a pharmacology study in the ISS model in immunosuppressed SD rats, in which no NR1 cells were detected in the brain in either study at 9 weeks and 3 months post-transplant, are also presented in this table.

The toxicology study using the athymic nude rat was designed to evaluate tumorigenicity and injection site persistence of NR1, as well general toxicology endpoints including extensive histopathological evaluations. Nine treatment groups of naïve (non-stroked) male and female nude rats per group received IC transplants of cGMP NR1 cells or a combination of NR1 cells and H9 cells. Cells were stereotactically infused into two (one cortex and one striatum) injection sites per hemisphere of the brain. Matrigel, a heterogeneous mixture of structural proteins and cellular growth factors, was included in the cell medium to enhance the likelihood of cell survival and growth of teratomas. The use of groups that received combinations of NR1 and H9 cells, i.e., that were spiked with H9 cells, were included to show that the athymic rat model supports the development of H9 stem cell-derived teratomas. These H9-spiked groups showed a linear dose-related formation of teratomas ranging from ˜67% of the treated animals at the high H9-spiked-NR1 doses (50%) to approximately 5% (1/19) of the animals treated with low H9 spiked NR1 doses (0.1%), respectively. The overall role of Matrigel, which was included in the cell medium used in the toxicology study, in promoting the growth of teratomas is not entirely understood. However, it is clear that the use of Matrigel supports the growth and persistence of NR1 without producing teratomas or toxicity, as shown in studies in which cell persistence was compared in the presence and absence of Matrigel. Matrigel is a complex protein mixture derived from mouse Engelbreth-Holm-Swarm sarcoma cells, that is reported to contain murine growth factors. Matrigel was only used to promote survival of the human cells in rodents and will not be included in the injection medium of the clinical NR1 drug product.

Administration of NR1 at doses up to 2×10⁶ NR1 cells IC or 1×10⁷ cells injected IM was not associated with any unexpected mortality, clinical findings, changes in body weight or food consumption, changes in clinical pathology or bone marrow endpoints, or macroscopic or microscopic observations. There were no NR1-related early deaths and/or euthanasia and no teratoma formation was seen in the brain or in peripheral tissues in any NR1-only group. NR1 was well tolerated at the low and high dose, 4×10⁵ and 2×10⁶ cells/animal, respectively, for as long as 180 days post-dose. No cells or teratomas were detected when H9 cells alone were administered IC into the brain or when NR1 or NR1 spiked with H9 cells were injected IM. In the H9 only-treated groups, cavitation was noted at the site of administration in the brain, suggesting the cells did not survive or engraft. The reason for the lack of growth of H9 cells in the brain when administered alone at a dose of 1×10⁶ is not clear. It was not due lack of viability of the H9 cell lot used, however, since an equal number of H9 cells spiked into 1×10⁶ NR1 cells resulted in the formation of teratomas (Group 4), as did the lower numbers of H9 cells used in the Groups 5-8. Rats that received IC injections of 1×10⁶ H9 cells/animal in a different cell medium, DMEM/F12 plus 0.5% HSA, with 30% rather than 40% Matrigel, developed visible teratomas by approximately 50 days post-treatment. The lack of persistence of the IM-injected cells NR1 and NR1+H9 at doses up to 1×10⁷ was likely due to a less permissive growth environment at the IM injection site.

No teratomas were detected in the brain or in peripheral tissues in any rat treated with NR1 cells only. A total of 100 athymic nude rats received up to 2×10⁶ NR1 cells per animal and were followed for up to 6 months post-transplantation. The model was able to detect teratomas at H9 levels as low 0.1% H9 cells (2000 H9 cells added to a dose of 2×10⁶ NR1 cells/animal) in prepared mixtures of NR1 and H9 cells. NR1 drug product is known to contain <0.01% H9 cells. The assay sensitivity for detection of hESC in NR1 is at least 10-fold below the lowest H9 hESC spiked level of 0.1% where only 1/19 animals developed an uncategorized microscopic tissue abnormality, compared to 67% of the 50% H9 hESC-spiked NR1 treated animals, where teratomas were observed in 67% of the animals and most animals did not survive for the entire duration of the study.

### Overview of the Nonclinical Study Results

The target for administration of NR1 was the subcortical portion of the brain tissue adjacent to the subcortical stroke, a region shown to be active in tissue repair and reorganization in functional human brain imaging and preclinical studies.

Biodistribution studies, noted above, were conducted and demonstrated that NR1 remains at the intracerebral injection site. The biodistribution of NR1 cells after transplantation, both within the brain and to peripheral tissues, is important to the assessment of safety. Additionally, an assessment of NR1 persistence in the brain offered information on the mechanism of NR1 action. A GLP-compliant 2-month biodistribution study after transplantation of NR1 cells to the peri-infarct area of immunosuppressed Sprague Dawley (SD) rats in middle cerebral artery occlusion ISS model was conducted. This model was also used in Study USF-1, which showed the efficacy of NR1 transplantation in partial recovery of motor and behavior function. This model is a well-characterized and is routinely used in the stroke field, providing the closest match to the proposed target indication of subjects with chronic ISS.

The presence and level of NR1 cells were quantified in the brain and neural axis, as well as selected peripheral tissues, gross lesions and tissue masses using a qualified and validated real time qPCR assay to detect human nuclear DNA and immunohistochemical (IHC) staining of tissues with the human anti-nuclear antibody HuNu. The NR1 used in the GLP-compliant biodistribution and toxicology studies was a cGMP product manufactured in compliance with cGMP and was also the product used in Study USF-1.

The biodistribution results showed that after injection into the parenchyma of the cortex and striatum of the brain at total doses of up to 4×10⁵ cells per animal (high dose), NR1 cells persisted within the brain for at least 45 days in acute and chronic stroke animals. The NR1 cells remained localized at the site of administration, with no migration away from the injection track to adjacent areas of the brain (with the possible exception of cells inadvertently injected into a ventricle) or to peripheral non-CNS tissues. Assessment of the persistence of NR1 cells at the injection site in the GLP toxicology study was consistent. Immunohistochemical staining (IHC) staining showed that the cells remained present at consistent low levels from 30 to 180 days after transplantation.

Cell persistence studies were also conducted in the GLP toxicology study, but in the presence of Matrigel. Using both IHC and qPCR analysis NR1 cells were detected at minimal to moderate levels in most treated animals as long as 180 days post-treatment. As in the biodistribution study, NR1 cells were present at the injection sites, visualized as cavitated spaces microscopically, within the brain parenchyma or less frequently within the ventricular spaces, with no evidence of migration of NR1 cells away from the injection sites. The occasional presence of NR1 cells in the ventricles was likely due to inadvertent injection of the cells into the ventricles during transplantation.

Overall, the results of multiple cell distribution or persistence studies showed the persistence of intracerebrally transplanted NR1 cells in the brain for up to 180 days after injection. These findings and the results of the two pharmacology studies that showed NR1-induced sustained partial recovery of general motor behavior, neurological function and motor coordination as measured by several different assessments, lasted as long as 3 months post-transplant, the end of the longest study provided encouraging results for stroke treatment. The NR1 biodistribution studies clearly showed no migration of NR1 cells away from the site of the intracerebral injection. When occasional, inconsistent, individual findings of NR1 cells in the ventricle were observed, they appeared to be the result of either inadvertent injection of cells into the ventricle given the small and delicate area for administration in the rodent brain (which does not occur in the clinical use of NR1 where the area is much larger) or due to contamination during collection.

Persistence of NR1 in the brain at 30 and 180 days after transplantation was clearly demonstrated in the GLP toxicology study which was conducted in the selected animal model, immunodeficient (athymic) naïve (non-stroked) nude rats. Animals received doses of 4×10⁵ NR1 cells (low-dose) or 2×10⁶ NR1 cells (high-dose). Microscopic examination of IHC-stained slides showed that NR1 cells were present at minimal to moderate levels at 30 days post-NR1 dose in the low-dose (17/20) and high-dose (16/20) animals. At the Day 180 necropsy (6 months post-transplantation), NR1 cells were present at minimal to marked levels in the low-dose (22/30) and high-dose (23/30) animals at 180 days post-dose. At both Days 30 and 180 post-transplant NR1 cells were mostly present at the injection sites, visualized as cavitated spaces microscopically, within the brain parenchyma or less frequently within the ventricular spaces, with no evidence of migration of NR1 cells away from the injection sites.

The GLP toxicology study, which was designed to demonstrate cell survival and lack of tumorigenicity/teratoma formation of NR1 in the athymic nude rat model, also showed that the athymic nude rat model used in the study would support the development of stem cell-derived teratomas in this model, by including treatment groups that also contained specified amounts of added H9 hESC to NR1. These H9-spiked groups showed, not only teratoma formation, but also showed decreasing, dose-related formation of teratomas associated with level of H9 cells added, ranging from ˜67% (˜14 in 20) to nearly 5% (˜1 in 19) of the treated animals at the high H9 (50%) and low H9 (0.1%) spiked doses, respectively. The overall role of Matrigel in promoting the growth of teratomas is not entirely understood, but it is clear that the matrix also supports the growth and persistence of NR1 without producing teratomas or toxicity, as shown in studies in which cell persistence was compared in the presence and absence of Matrigel. However, even in the absence of Matrigel, NR1 cells survived in the brain for as long as 45 days post-transplant with the same outcome.

No cells or teratomas were detected when NR1 cells were injected into the brain or when NR1 cells spiked with H9 cells were injected IM. No cells or teratomas were detected when H9 cells alone were administered IC into the brain. No H9 cells were detected at one-hundred-eighty days after H9 only cell injection. Cavitation noted at the site of administration in the brain suggested the cells did not survive or engraft. The reason for the lack of growth of H9 cells in the brain when administered alone (unspiked with NR1) at a dose of 1×10⁶ is not clear. It was not due to lack of viability of the H9 cell lot used, however, since an equal number of H9 cells spiked into 1×10⁶ NR1 cells did result in the formation of teratomas, as did the lower numbers of H9 cells used in the Groups 5-8. Rats that received IC injections of 1×10⁶ H9 cells/animal in a different cell medium, DMEM/F12 plus 0.5% HSA, with 30% rather than 40% Matrigel developed visible teratomas by approximately 50 days post-treatment. The lack of persistence of the IM-injected cells (NR1, NR1+H9) at doses up to 1×10⁷ was likely due to a less permissive growth environment at the IM injection site.

In the GLP long term toxicology study conducted with NR1, no tumors were detected in NR1 treated animals. 50 male and 50 female athymic nude rats received up to 2×10⁶ NR1 cells per animal and were followed for up to 6 months post-transplantation. Neither local nor systemic toxicity occurred after treatment. NR1 engrafted and persisted for the duration of the study, NR1 did not distribute or migrate from the site of administration and no tumors or teratomas were formed in the brain or peripheral tissues.

The animal model for showing tumors/teratomas was validated by showing that addition of H9 hESC to NR1 at escalating dose levels did produce dose-related tumors with increasing H9 percentage. The assay sensitivity for detection of hESC in NR1 is at least 10-fold below the lowest H9 hESC spiked level of 0.1% where only 1 animal (˜5%) showed an uncategorized microscopic tissue abnormality, compared with 67% of the 50% H9 hESC-spiked NR1-treated animals, where teratomas were observed and animal survival for the duration of the study was an issue. See graph, below:

No tumors were observed with NR1 injected animals, consistent with all the toxicology evaluations. As H9 spiked levels increased and NR1 levels decreased, the treated animals developed tumors. Thus, the animal model is valid for showing tumors with H9 down to 0.1%, and that NR1 drug product itself does not produce tumors. This study, in addition to the other studies cited above, supports the safety of NR1.

### Relevance of Nonclinical Studies; Dose Extrapolation to Humans

The administration target for NR1 is the subcortical portion of the brain tissue adjacent to the subcortical stroke, a region shown to be active in tissue repair and reorganization in functional human brain imaging and nonclinical studies. This administration is consistent with nonclinical pharmacology studies in which injection coordinates were identified to directly deliver NR1 into tissue adjacent to the stroke lesion in rodent models of subcortical and cortical ischemic stroke, based on prior determination of the average size and location of lesions in these respective models. In this nonclinical model, the NR1 treated animals showed improved function.

The proposed clinical study dose escalation subject cohorts and their respective doses are shown in the table below:

TABLE 1

| | Dose | | Volume per | Total | Cell Concentration at |
|---|---|---|---|---|---|
| Cohort | (Cells) | Injections | Track | Volume | dosing (cells/mL) |
| 1 | 2.5 × 10⁶ | 3 | 100 μL | 300 μL | 8.3 × 106 |
| 2 | 5.0 × 10⁶ | 3 | 100 μL | 300 μL | 16.6 × 10⁶ |
| 3 | 10 × 10⁶ | 3 | 100 μL | 300 μL | 33.3 × 10⁶ |
| 4 | 20 × 10⁶ | 3 | 100 μL | 300 μL | 66.6 × 10⁶ |

In nonclinical models, doses of up to 2×10⁶ cells were evaluated in the toxicology study. Based upon a rat-to-human striatal volume ratio of 1:74, a corresponding equivalent clinical dose of 148×10⁶ cells may be extrapolated as a potential maximum dose. However, the first clinical trial dose proposed, adjusting for a 60-fold safety margin based on striatal volume ratio, is 2.5×10⁶ cells/mL. Subsequent dose escalation to 5.0×10⁶, 10×10⁶ or 20×10⁶ NR1 cells (safety margins of 30-fold, 15-fold, and 7.5-fold) per fixed volume of 1 mL is proposed. This is comparable to the doses and concentrations used in other stem cell clinical trials for stroke using intracerebral delivery, and is within the extrapolated dose range for which efficacy was observed in nonclinical pharmacology studies in which 1×10⁵ and 4×10⁵ cells were efficacious in rodents. Using the rat-to-human striatal ratio of 1:74, the rat dose of 1×10⁵ and 4×10⁵ cells would scale to clinical doses of 7.4 million and 29.6 million cells respectively.

These conclusions of ability to administer NR1 safely in humans are based on data from the extensive testing outlined above, with the following numbers of adult athymic nude rats tested, which provide sufficient data and results to calculate the safety margin determinations.
There were 382 adult athymic nude rats treated with intracerebral NR1. Of these 382 adult athymic nude rats, 102 animals were treated with IC NR1 at a dose of 2×10⁶ cells/animal and 24 adult athymic nude rats treated with IC NR1 at a dose of 1×10⁶/animal.
Sixty (60) of the adults athymic nude rats were followed for 180 days post-treatment, where half of these animals received 2×10⁶ cells, and half received 4×10⁵ cells.
Adult athymic nude (40) rats were treated with intramuscular NR1 at 1×10⁷ cells/animal with follow-up to 180 days, and there was no migration of cells or extracellular growth in these animals.
Neonatal athymic nude (6) rats were treated with intracerebral NR1 at 1×10⁶ cells/animal.

NSG mice (6) were treated with intracerebral NR1 at 1×10⁶ cells/animal.

The high-dose animals were treated at the maximum feasible dose, and sacrifice time points were selected at 30 and 180 days after dosing. The long follow-up duration (6 months) was included in the GLP toxicology study to allow time to detect the presence of slow-forming teratomas and ectopic tissue masses that could be caused by the growth of NR1 cells or contaminating stem cell precursors. The cell injection medium contained Matrigel®, which is known to promote the survival of stem cell-derived neural progenitors in somatic tissues to enhance NR1 cell growth and optimize detection of the rare formation of teratomas. No local or systemic toxicity was seen after treatment with NR1, including any effects on survival, clinical pathology endpoints and microscopic findings in the tissues. No evidence of tumor or teratoma formation was detected after NR1 treatment in any tissue or organ during the 6-month duration of the study. IHC staining and qPCR analysis showed that the cells remained at low, but consistent levels from 30 to 180 days after transplantation and did not migrate from the site of injection. NR1 persistence has been shown and lack of migration in several other studies, including the pilot toxicology studies and the two pharmacology studies. The NR1 biodistribution studies also showed no migration of NR1 cells away from the site of the intracerebral injection.

A biodistribution study was conducted after transplantation of NR1 cells to the peri-infarct area of immunosuppressed Sprague Dawley (SD) rats in the middle cerebral artery occlusion ISS model. This model was also used in Study USF-1, the primary pharmacology study with NR1 that showed the efficacy of NR1 at inducing partial recovery of motor and behavior function. The model is a well-characterized ISS model that is routinely used in the stroke field and provides the closest match to the proposed target indication of subjects with chronic ISS.

After injection into the cortex and striatum of the brain at doses of 4×10⁵ cells per animal in the biodistribution study, NR1 cells persisted at the peri-infarct injection site for at least 45 days. NR1 cells remained localized at the site of administration, with no migration away from the injection tract to adjacent areas of the brain or to peripheral tissues. Findings in the GLP toxicology study, in non-stroked athymic nude rats confirmed these findings, with no migration of NR1 cells away from the injection track and long-term persistence of cells at the injection site in the brain.

### Preponderance of the Evidence Supporting Safe Administration of NR1 in the Clinical Study

The pilot pharmacology studies, the 60-day GLP biodistribution study in the ISS rat model in immunosuppressed SD rats, and the 6-month GLP toxicology study in athymic nude rats were all evaluated to assess the long-term safety of a single IC dose of NR1 at levels up to 2×10⁶ cells per animal. The preponderance of evidence supporting safe administration of NR1 clinically include:
The validated athymic nude rat model showed, through spiking NR1 with H9, that contamination of precursors would detect teratomas if the h9 hESC cells were present in sufficient quantities;
The long follow-up duration provided time to detect the presence of slow-forming teratomas and ectopic tissue masses that could be attributed to the NR1;
The cell injection medium used in the toxicology studies contained Matrigel®, which is known to promote the survival duration of stem cell-derived neural progenitors in somatic tissues (Akbasak et al., 1996; Jin et al., 2010a; Jin et al., 2010b; Kutschka et al., 2006), to promote NR1 cell growth and optimize detection of the formation of teratomas; and
The lack of local or systemic toxicity after prolonged treatment with NR1, including no effects on survival, clinical pathology endpoints or microscopic findings in the tissue.

The above conclusions provide confidence that the NR1 doses used in the animal model can be safety administered IC to the brain, as no evidence of toxicity, tumor or teratoma formation was detected after NR1 treatment in any tissue or organ during the 6-month duration of the study. NR1 did not distribute or migrate from the site of administration, with no evidence of human cells at any distance from the brain injection site or in other tissues of the body. The presence of NR1 cells at the injection site was demonstrated using a very sensitive assay that detects human DNA and by immunohistochemical staining of human cells on microscope slides, with detectable cells evident at the peri-infarct injection site as long as 180 days after transplantation.

In nonclinical models, doses of up to 2×10⁶ cells were evaluated in stroke model animals. As described above, stroked animals treated with up to 2×10⁶ cells/mL regained some pre-stroke motor function.

Based upon a rat-to-human striatal volume ratio of 1:74, a corresponding equivalent clinical dose of 148×10⁶ cells may be extrapolated as a potential maximum dose. However, the first clinical trial dose proposed, adjusting for a 60-fold safety margin based on striatal volume ratio, is 2.5×10⁶ cells/mL. Subsequent dose escalation to 5.0×10⁶, 10×10⁶ or 20×10⁶ NR1 cells (safety margins of 30-fold, 15-fold, and 7.5-fold) per fixed volume of 1 mL is proposed. This is comparable to the doses and concentrations used in other stem cell clinical trials for stroke using intracerebral delivery and is within the extrapolated dose range for which efficacy was observed in nonclinical pharmacology studies in which 1×10⁵ and 4×10⁵ cells were efficacious in rodents. Using the rat-to-human striatal ratio of 1:74, the rat dose of 1×10⁵ and 4×10⁵ cells would scale to clinical doses of 7.4 million and 29.6 million cells respectively.

### Example 2

### Non-Clinical Overview

NR1 drug product is a neural stem cell product derived from the human embryonic stem cell (hESC) H9. It is being developed for treatment of chronic ischemic subcortical stroke with motor deficits in adult subjects with hemiparesis. The target for administration of NR1 is the subcortical portion of the brain tissue adjacent to the subcortical stroke, a region shown to be active in tissue repair and reorganization in functional human brain imaging and nonclinical studies. Two nonclinical studies were conducted to test the efficacy of NR1 treatment, one in a model of ischemic subcortical stroke (ISS) in immunosuppressed SD rats and one in a model of ischemic cortical stroke (ICS) in immunodeficient athymic nude rats. Immunodeficient or immunosuppressed animals were used to minimize the potential rejection of the human NR1 cells. Investigational studies were conducted to help elucidate the mechanism of action of the NR1-associated partial sensorimotor recovery seen in both efficacy models and to demonstrate that appropriate animal models were used in the GLP biodistribution and toxicology studies.

Because of the cellular nature of the NR1 drug product, pharmacokinetic, metabolism or excretion studies were not applicable. GLP biodistribution studies were conducted after intracerebral (IC) injection of NR1 in the ISS stroke model in immunosuppressed SD rats (which is also the model used in the pivotal pharmacology study). Naïve (non-stroked) athymic nude rats in the pivotal GLP toxicology study. The GLP toxicology study evaluated the potential adverse effects of IC injection of NR1 over a period of 180 days after transplantation. It also included treatment groups that received mixtures of NR1 drug product and H9 hESC at levels up to 50% of the NR1 treatment dose to determine the ability of the immunodeficient rat model to detect teratomas that might develop after treatment. Matrigel®, a heterogeneous mixture of structural proteins and cellular growth factors, was included in the cell medium of the toxicology studies to enhance the likelihood that teratomas would form if contaminating H9 cells remained in the NR1 drug product. Matrigel will not be used in the clinical studies in human subjects, and it was not used in the nonclinical pharmacology or GLP biodistribution studies.

Some of the studies conducted to assess the efficacy, biodistribution and toxicology of NR1 were “combined studies”, i.e., their study endpoints included multiple of the aforementioned endpoints with data collected concurrently. For example, a study might include pharmacology and toxicology endpoints, but no biodistribution endpoints, or might include all three areas. The assessment of multidisciplinary endpoints in a single study permit an integrated assessment of the interrelationships of activity and toxicity in the same animal model system, allowing a unique insight into the relationship of efficacy, activity and biodistribution, as well as the safety. Questions such as whether NR1 cells are distributed outside the brain or if NR1 cells have prolonged survival can offer a roadmap to unexpected target organs for toxicity. This integrated nonclinical testing strategy provides the most relevant nonclinical assessment of efficacy and safety of NR1 cell therapy and extrapolation to the clinical setting.

### Primary Pharmacodynamics

Two non-GLP pharmacology studies were performed to test the efficacy and safety of IC NR1 treatment in nonclinical rat models of ischemic subcortical stroke (ISS) and ischemic cortical stroke (ICS). Functional recovery was measured using a battery of motor and behavioral tests to quantify post-injury recovery of sensorimotor function. Histological evaluations were used to explore candidate mechanisms of action for potential correlation with NR1 effects in both studies. In both pharmacology studies, the evaluation of potential toxicity was limited to recording of clinical signs of adverse effects, body weight changes and survival to avoid inference with the assessment of recovery. Several small investigational studies were conducted to evaluate the mechanism of action of NR1 drug product. These specialized studies evaluated the role of cell survival, inflammation, microglial and natural killer cells, neural plasticity and inflammation on NR1-related sensorimotor recovery, performed a secretome analysis of NR1 and examined the use of TSPO-PET as a predictor of NR1-induced functional recovery post-stroke.

The pivotal efficacy study evaluated the potential therapeutic value of intracerebral (IC) transplantation of NR1 in immunosuppressed Sprague-Dawley (SD) rats in the middle cerebral artery occlusion model of ISS. This system is a well-characterized ISS model that is routinely used in the stroke field and provides the closest match to the proposed target indication of human subjects with chronic ISS. The primary endpoint of the study was to test the hypothesis that NR1 treatment at either 7 or 28 days post-stroke would result in a significant improvement in the elevated body swing test (EBST), with secondary endpoints of improvements in the neurological exam and the Rotorod test. It showed that IC transplantation of NR1 in a model of ISS promoted recovery of general motor behavior, neurological function and motor coordination. The onset of functional recovery across tests occurred at month 2 post-transplantation and persisted up to at least 3 months post-transplantation (the end of the study). Overall, transplantation at both the acute and chronic phase of stroke and at both low and high cell doses significantly promoted persistent functional recovery, but transplantation at the chronic phase and at the high dose appeared to be the most optimal transplant regimen based on EBST and Rotorod Test results.

Histological analyses confirmed that the ischemic injury was unilateral and that the most pronounced functional losses were observed in locomotor function and motor coordination. Histological assessment at 3 months post-treatment suggested a link between increased peri-infarct blood vessel density and NR1-associated enhancement of functional recovery when NR1 was administered during the acute phase (7 days) post-stroke and between decreased peri-infarct microglial/immune cell activation and NR1-associated enhancement of functional recovery in the high-dose NR1 in the chronic phase (28 days) post-stroke. While it is likely that additional mechanisms of host brain repair also contribute to the observed effects on functional recovery, neovascularization and immunomodulation warrant further investigation as candidate mechanisms of action of NR1. Although no surviving NR1 cells were found at or near the injection site 3 months after transplantation, improvements in functional recovery were observed beginning at 2 months post-treatment and persisting until at least 3 months post-treatment (study end), indicating that chronic recovery enhancement occurred in the apparent absence of long-term NR1 persistence.

A supporting efficacy study evaluated the therapeutic effect of IC transplantation of NR1 in athymic nude rats using the ICS model, another widely used stroke model. Sensorimotor function testing began 1 week prior to stroke injury and then weekly until 8 weeks post-treatment. Following completion of the in-life assessments, brain tissue was collected at 9 weeks post-treatment for histological evaluation. The primary measure of functional recovery in Stanford-5 was a sensorimotor behavior test known as the Vibrissae-Paw Test. Secondary testing modalities and histological assessment were also included. The results of the testing showed treatment with the high dose, but not the low dose, of NR1 cells improved sensorimotor function. Improvements, first seen at 4 weeks post-treatment, persisted until the end of the study end. Histological assessments showed that high dose-treated rats had reduced microglial/immune cell activation in peri-infarct cortex relative to vehicle controls at 9 weeks post-treatment, but which did not correlate with functional recovery. No significant differences in infarct size, peri-infarct blood vessel density, or peri-infarct axon sparing/outgrowth were observed with either NR1 dose relative to vehicle treatment. No surviving human cells (NR1 cells) were detected in any animal at 9 weeks post-administration, suggesting that NR1 cells were unable to persist long-term in this experiment in this xenograft injury model.

Several specialized investigational studies were conducted to examine the effect of NR1 cells in stimulating functional recovery after stroke in animals, as well as to determine the optimal way to increase the survival of the human NR1 cells in rodents. Studies included evaluations of the relative role of inflammation and cellular plasticity on functional recovery, a secretome analysis of transplanted NR1 cells, an evaluation of the effect of depletion of NK or microglial cells on NR1 cell persistence and whether the T2-FLAIR signal could serve as a predictor of NR1 cell-associated functional recovery and assess the immunomodulatory function of the treatment.

We assessed the role of the inflammatory response triggered by NR1 transplantation into stroked rat brains. Although NR1-treated animals exhibited a more variable immune response than control animals, the immune response early after transplantation by NR1 changing macrophages from an M1/pro-inflammatory state to an M2/anti-inflammatory state, which correlated with the result of in vitro plasticity studies. To further evaluate the immunomodulatory effects of NR1 transplantation in chronic stroke the T2-FLAIR MRI signal was used in a rat stroke model as a potential clinical predictor of NR1-induced functional recovery in stroke subjects. The T2-MRI FLAIR signal is often associated with inflammation. It and a second non-invasive imaging modality, PET imaging of the translocator protein 18 kDa (TSPO), called TSPO-PET, were used to monitor brain inflammation in rats. TSPO comprises activated immune cells including brain resident immune cells, microglia and astrocytes, and infiltrating myeloid cells, such as monocytes/macrophages, neutrophils and dendritic cells. TSPO levels are low in the healthy brain but upregulated under inflammatory conditions. TSPO-PET radioligands thus serve as a useful index of neuroinflammation, and increased TSPO-PET signals have been observed in stroke subjects, both in the stroke lesion and in remote brain areas at times ranging from 5 days to 24 months after stroke. Immunohistochemical analysis of the needle track region associated with the FLAIR lesion showed qualitative differences in the inflammatory response between NR1- and vehicle-treated animals, where treated animals have more activated macrophages/microglia on the border of the injection track. TSPO-PET data from stroked nude rats were consistent with the FLAIR findings, with higher immune cell activity near the transplantation site and also more distant in NR1-treated compared to vehicle-treated rats. Much of the immune cell activity occurred in connected regions of the brain, implying widespread immunomodulatory effects of the hNSCs. The results of this study, particularly considered in concert with the in vivo secretome analysis done in NR1-treated rats discussed below, strongly implicate immunomodulation as a major mechanism of action of NR1 facilitating stroke recovery.

Two studies were conducted that evaluated the role of secreted factors in the activity of NR1. One assessed the effect of NR1 cells on neural plasticity by analyzing how NR1 cells affect cortical activity in the peri-infarct area of nude rats after stroke. Brain plasticity, or rewiring, plays a key role in the spontaneous recovery observed after stroke in humans and rodents, with rewiring occurring in the surviving circuits adjacent to the peri-infarct zone, which enables healthy brain areas to compensate for the functions of damaged areas. NR1 transplantation significantly (p<0.05) increased motor circuit activity in the peri-infarct cortex at Day 7 after transplantation, most prominently in cortical layers 2 and 3. Conversely, inhibitory connections were significantly (p<0.05) reduced in magnitude and delayed in onset. No significant effects of NR1 on excitatory connections were seen. These findings suggest that NR1 cells increase motor circuit activity by releasing individual neurons in the peri-infarct region from inhibitory control. In vitro studies showed that NR1 cells affected the plasticity of neuronal cells, specifically by enhancing synaptogenesis of retinal ganglion neuronal cells (RGCs). Gene ontology analysis linked NR1 transplantation with the expression of genes associated with plasticity, suggesting that NR1-related early modulation neuronal activity and plasticity at an early stage results in late functional recovery. NR1 cells were found to cause a specific and differential expression of genes involved in stimulating adult neural precursor cell proliferation, neuronal differentiation, gated channels directly involved in the electrophysiological properties of neurons, axon guidance, axonogensis, and synaptogenesis. Overall, it appears that NR1 cells contribute to the plasticity of peri-infarct regions after stroke by releasing individual neurons in the peri-infarct cortex from inhibitory control, as well as by modulating plasticity by secreting factors that stimulate synaptogenesis. A secretome analysis of NR1 cells used TRAP analysis and RNA sequencing (TRAP/RNAseq) 2 days after stroked rats received GFP-labeled NR1. It identified 175 genes for secreted proteins, with the top 20 enriched processes associated with brain repair, including extracellular matrix (ECM) remodeling (involved in most brain repair mechanisms) and processes related to inflammation and axon guidance. Again, NR1-secreted factors appear to have a role in modulation of brain repair and axon guidance. In vitro analysis identified ˜400 genes that encode for secreted factors, with nearly 200 associated with inflammation, primarily cytokine activity, and others involved in extracellular matrix (ECM) organization. Specifically, NRG3 and TGFb3 were upregulated in the stroke environment in vivo. Overall, these two studies demonstrated that NR1 cells secrete factors that can modulate a variety of other factors that are in involved in late functional recovery in the brain.

In an effort to improve the animal models for the biodistribution and toxicology studies, two studies two studies were conducted to evaluate factors involved in the survival of NR1 cells in the brain after transplantation. The impact of decreasing infarct microglial/monocyte cell activation during the chronic phase (28 days) after ISS induction was suggested by the outcome of histological analyses. Because microglia mediate immune responses in the CNS by acting as macrophages, they (microglia) may have a deleterious effect on the survival of NR1 after transplantation. Using PX5622 in the feed, which depletes microglial cells, microglia numbers were decreased by up to 40% in the brain and 52% in the blood in the week after transplantation. No effect on NR1 cell survival was seen one week after transplantation. A second study examined the impact on NR1 survival of depleting Natural Killer (NK) cell numbers by treatment with anti-asialo/GM1 antibodies before and after NR1 transplantation. As in the previous study, NR1 survival was not improved, despite depletion of NK levels by ˜80-90%. Only NR1 survival was measured in both these studies, not direct effects on stroke recovery, and it is possible that the complexity of the recovery process may impair our understanding of the relationship of NR1 cell survival to stroke recovery. A secondary conclusion was that neither depletion of microglial nor NK cells would result in improved NR1 survival and result in a better animal model for testing the efficacy and safety of NR1.

### Analytical Methods

A quantitative real-time PCR (qPCR) method was developed by Charles River Laboratories, Mattawan (CRL-MTWN, formerly MPI, Inc.) for quantification of levels of human nuclear gDNA in rodent tissues. The assay was fully qualified and validated for use in the GLP biodistribution and toxicology studies in rats. The validated assay was cross-validated to permit the use of DNA from HK293 cells to prepare a standard curve for NR1 gDNA in fixed and frozen rat tissue from Study USF-1. The primer and probe sequence for detection of human nuclear DNA were the same in all the assays utilized in these studies.

Total DNA was extracted from rat tissues per MPI SOPs CMB-19 and CMB-78. qPCR reactions were performed on 96-well plates following CMB method, CMB-2122-007-B. Each plate was run with a standard curve, a set of QC samples and the study samples. The standard curve was run in 3 replicates for the standard points at 10⁴, 10⁵, 10³, 2×10², 10², 50, 20, and 0 (zero) copies per reaction. Standards with zero copies, containing only matrix, were included to confirm reagent purity.

Concentration limits were as follows: Limit of detection (LOD): 10 copies of gDNA/μg of tissue; samples below the LOD were identified as BLOD; Lower limit of quantification (LLOQ): 20 copies of gDNA/μg of tissue; samples that gave results between 10 and 20 copies per reaction were identified as BLOQ; Upper limit of quantification (ULOQ): 10⁴ copies of gDNA
The qPCR method was fully qualified and validated.
Absorption
No absorption studies were conducted with NR1.
Distribution
No protein binding, distribution or placental transfer studies with done with NR1. Biodistribution was evaluated in several studies.

A GLP-compliant two-month biodistribution study was conducted in immunosuppressed normal rats that underwent an ischemic subcortical stroke (ISS) procedure followed by IC treatment with NR1. Levels of NR1 were quantified using a validated qPCR assay at multiple time points up to 60 days post-transplant. Additional studies on cell persistence and distribution were conducted as part of the GLP toxicology study. Three (3) months post-transplant selected tissues were collected at necropsy from SD rats (USF-1) that had undergone an ISS for quantification of NR1 cells for quantification of NR1 cells using IHC and/or qPCR. Similarly, selected tissues were collected 9 weeks post-transplant from athymic nude rats that had undergone an ICS for quantification of NR1.

We monitored the distribution of test article, NR1, transplanted into the brain of “stroke model” rats. In this model, an ISS was induced surgically by middle cerebral artery occlusion and NR1 cells were injected IC either 3 to 5 days post-stroke (acute stroke) or 28 to 30 days post-stroke (chronic stroke). Rats were immunosuppressed with intraperitoneal injections of cyclosporine A to decrease the likelihood of immune rejection of the human NR1 cells. At time points ranging from 24 hours to 60 days post transplantation slides were prepared from the brain and selected peripheral tissues for microscopic examination using H&E staining and IHC staining to identify human cells. Samples of non-CNS tissues (as listed above) and brain tissue were collected using strict aseptic technique for analysis with a validated real time qPCR assay to detect human nuclear DNA. IHC showed the occurrence of NR1 at minimal to minimal to moderate magnitude of NR1 cells in brains of animals in both the acute and chronic stroke groups as long as 45 days post-transplant with a general decreasing trend in both incidence and magnitude. The presence of NR1 cells was limited to the hemisphere of the brain into which the cells were injected, and cells were not detected in any other organs examined in any of the rats.

TABLE 2
Persistence of NR1 in the Brain (IHC Determination)

| Necropsy Day | | | | | |
|---|---|---|---|---|---|
| 1 | 4 | 8 | 30 | 45 | 60 |

Acute Stroke (NR1 transplant on Days 3-5 post-stroke)

| | | | | | | |
|---|---|---|---|---|---|---|
| NR1 cells | ND | 7/7 | 5/6 | 3/8 | 8/10 | 1/10 |
| present/no. | | | | | | |
| animals | | | | | | |
| examined | | | | | | |
| minimal | — | 2 | 5 | 3 | 7 | 1 |
| mild | — | 5 | 0 | 3 | 1 | 0 |
| moderate | — | 0 | 0 | 0 | 0 | 0 |

Chronic Stroke (NR1 transplant on Days 28-30 post-stroke)

| | | | | | | |
|---|---|---|---|---|---|---|
| NR1 cells | 10/10 | 9/10 | 4/10 | 2/10 | 4/9 | 0/10 |
| present/no. | | | | | | |
| animals | | | | | | |
| examined | | | | | | |
| minimal | 5 | 2 | 3 | 2 | 4 | 0 |
| mild | 0 | 5 | 1 | 0 | 0 | 0 |
| moderate | 5 | 2 | 0 | 0 | 0 | 0 |

NR1 cell gDNA was not detected in any of the brain samples from animals in the acute stroke group using the qPCR assay most likely due to the tissue sampling procedures in which the tissues nearest the injection site were used for histopathology studies, with only more distant tissue used for qPCR. In contrast, tissues at and near the injection site were collected for both analyses in the chronic stroke group. In this case, NR1 cell gDNA was detected in 6/10 animals 24 hours post-dose (copy numbers ranged from 33 to 601 copies/μg sample DNA), in 2/10 animals 3 days post-dose (copy numbers of 68 and 91) and in 2/10 animals (both BLOQ) one week post-dose. No NR1 cells were detected at 30, 45 or 60 days post-dose using qPCR. The presence of NR1 gDNA at the early time points in the chronic groups compared to the acute group was likely due to the change in site of collection of tissues for qPCR analysis, not a true difference in NR1 cell survival.

No NR1 cells were detected in any area of the brain distant from the injection site or in peripheral tissues using either IHC or qPCR, with the possible exception of cells inadvertently injected into a brain ventricle in a few animals. NR1 cells were limited to the hemisphere of the brain and, specifically the injection track, in all rats in which transplanted NR1 cells could be identified microscopically by IHC staining. No NR1 cells were detected in the brain at sites distant from the injection site or in any peripheral organ. In six animals, very low levels of NR1 were detected in single spinal cord sections at levels near or below the limit of quantification of the qPCR assay (the highest copy number was 22 in a single animal, with BLOQ values in the other 5 animals). Spinal cord sections from the animals were submitted for IHC HuNu staining for human cells in evaluate this finding. Samples from all but one of the affected animals contained occasional HuNu-positive cells (single individual cells ranging from 1 to 3 cells per affected slide). All cells were either present bordering/external to the meninges of the spinal cord or were not present in the same plane of section as the spinal cord tissue sections. These findings did not indicate direct migration of NR1 cells into the spinal cord from the injection site, but most likely contamination from the surgical procedure related to the small working area of the cranium and brains of the rodents, inadvertent injection into the ventricular and subarachnoid CSF spaces, and/or introduction of occasional cells as a result of necropsy or tissue processing procedures. This will not be an issue with the human surgical procedure because of the ability to avoid contamination in the much larger human cranial surgical field compared that of the rat, thus eliminating inadvertent injection into the ventricular and subarachnoid CSF spaces. No evidence of teratomas was detected either macroscopically or microscopically in the brain or any other tissue.

Persistence of NR1 in the brain at 30 and 180 days after transplantation was also evaluated in the GLP toxicology study, which was conducted in immunodeficient naïve (non-stroked) nude rats. Using IHC staining, NR1 cells were seen to be present at minimal to moderate levels at 30 days post-NR1 dose in the low-dose (17/20) and high-dose (16/20) animals at or near the injection site, compared to minimal to marked levels in the low-dose (22/30) and high-dose (23/30) animals at 180 days post-dose at the 6 months after transplantation. As in the biodistribution study, NR1 cells were mostly present at the injection sites, visualized as cavitated spaces microscopically, within the brain parenchyma or less frequently within the ventricular spaces, with no evidence of migration of NR1 cells away from the injection sites. The presence of NR1 cells in the ventricles may have been due to inadvertent injection of the cells into the ventricle during transplantation (see comments above). qPCR analysis results were consistent: NR1 cells were present in the low- and high-dose IC injected animals at both 30 and 180 days after transplantation. At 30 days post-dose, a range of 21-54 copies of NR1 gDNA per μg of brain lysate gDNA (6/20 animals) was measured in at the low dose compared to a range of 21-37 copies at the high-dose (14/20 animals). By 180 days post-administration, 25-73 copies were detected in 17/20 low-dose animals compared to 25-97 copies in 18/20 high-dose animals. NR1 gDNA was not detected at the IM injection site in the Group 11 animals, which received 1×10⁷ NR1 cells, and no NR1 gDNA was detected in control animals.

Other studies evaluating NR1 persistence in the brain include three pilot toxicology studies in naïve (non-stroked) NSG mice or nude rats as well as in a small mechanism of action study conducted in naïve nude rats.

The largest of the studies evaluated the persistence of NR1 cells injected IC in naïve (non-stroked) nude rats in DMEM/F12 medium plus 0.5% HSA with or without 30% Matrigel. Cell persistence was followed for ˜88 days using semi-quantitative IHC staining for human nuclear protein antigen, HuNu. At 88 days, cells were detected at low levels (<0.2% of the original cell dose in 2/4 animals) only in the Matrigel-containing groups, with no detectable cells in the non-Matrigel group. Although much lower than the number of cells detected in the two GLP studies, the results also show prolonged survival of IC-transplanted NR1 cells. Two smaller studies also consistently showed low levels of survival for as long as 60 days post-dose, as well as improved NR1 survival when Matrigel was included in the cell medium compared to no Matrigel.

Overall, the results of multiple cell distribution or persistence studies showed the persistence of intracerebrally transplanted NR1 cells in the brain for up to 180 days after injection. These findings are considered in concert with the results of the two pharmacology studies that showed NR1-induced persistent partial recovery of general motor behavior, neurological function and motor coordination as measured by several different assessments, lasting as long as 3 months post-transplant, the end of the longest study. The NR1 biodistribution studies clearly showed no migration of NR1 cells away from the site of the intracerebral injection. Occasional findings of NR1 cells in the ventricle are believed to be the result of either inadvertent injection of cells into the ventricle (which will not occur in the clinical use of NR1 due the larger surgical field in human subjects) or to contamination during collection.

### Single-Dose Toxicology

Several single-dose studies NR1 toxicology studies were conducted in rodents, including three pilot studies and a 180-day GLP-compliant study. The GLP study was conducted using cGMP NR1 drug product in male and female naïve (non-stroked) athymic nude rats and had a duration of 180 days. Its primary objective was to evaluate the tolerability and general toxicity of the NR1 drug product. The study also evaluated the persistence and potential for NR1 cells to migrate from the site of injection over a 6-month time period after cell transplantation. In addition, the study design included groups of animals treated with NR1 cells spiked with H9 hESC at specific levels ranging from 0.1 to 50% of the 2×10⁶ cell NR1 dose (2×10³ to 1×10⁶ H9 cells/animal) to determine the ability of the athymic rat model to detect the formation of stem cell-related teratomas.

Prior to conducting the GLP toxicology study, pilot studies were conducted to select the optimal animal model and determined the suitability of three immunodeficient animal models, NSG mice, neonatal athymic nude rats and adult athymic nude rats over 28 days after transplantation. Based on its results, the adult athymic nude rat was selected for further evaluation. A second pilot further characterized the adult athymic nude rat model, following NR1 persistence in the brain for up to 2 months post-injection. It also assessed the effect of Matrigel, included in the cell medium to support cell survival, on NR1 persistence at the injection site. The earliest pilot study with NR1 was also conducted in adult nude rats but using a different cell medium (DMEM/F12 plus 0.5% HSA), rather than the Plasmalyte-A plus 0.5% HSA used in the pivotal toxicology study and in the two pilot studies described above). The DMEM/F12 medium was not used after this study as it was deemed not suitable for use in human subjects. This study also included H9 hESC to evaluate the suitability of the model and also evaluated the effect of Matrigel on cell persistence post-transplant. As in the other studies, NR1 was well tolerated. Limited NR1 persistence data was consistent with that later seen in the large GLP studies. Matrigel was included in the cell medium in three pilot studies to provide the optimal environment for cell growth to help to assess the survival of NR1 cells and the potential growth of teratomas. In the two studies in which the effect of including Matrigel was assessed, persistence of NR1 cells in the brain was improved by the inclusion of Matrigel.

The animal model and design of the GLP toxicology study was based on finding in the pilot studies that showed the beneficial effect of Matrigel on cell persistence in the brain after transplantation and the relatively increased persistence if NR1 cells in the adult athymic nude rat compared to neonatal nude rats or NSG mice. Two investigational studies that showed the lack of effect on NR1 persistence of reducing the number of microglial or NK cells, led to discarding these approaches for selecting the GLP study animal model. Based on preliminary work, the adult athymic nude rat, with no pre-treatment to selectively decrease specific cell populations, and including the addition of Matrigel to the cell medium to improve cell survival, was chosen for the GLP toxicology study.

In the GLP study, nine treatment groups of naïve (non-stroked) male and female nude rats per group received IC transplants of cGMP NR1 manufactured at the City of Hope National Medical Center, or H9 hESC as a positive control, or a combination of NR1 cells and H9 cells. Cells were stereotactically infused into two cortex and one striatum injection site per hemisphere of the brain. Matrigel, a heterogeneous mixture of structural proteins and cellular growth factors, was included in the cell medium to enhance the likelihood of cell survival and growth of teratomas. Three treatment groups received the test article or a combination of the positive control and test article via intramuscular (IM) injection into the biceps femoris of the left hindlimb. The dose were 4×10⁵ or 2×10⁶ IC or 1×10⁷ IM. Animals were maintained until approximately Day 30 or 180 for final necropsy. Combination groups comprised NR1 cell spiked with level of NR1 ranging from 50% (1×10⁶ H9 cells) to 0.1% (2000 H9 cells) of the 2×10⁶ NR1 dose. Standard toxicology endpoints were monitored. Necropsies were performed at approximately 30 and 180 days after transplantation of NR1. Slide were prepared from tissues and stained with H&E or underwent IHC staining to identify human cells. The left hemisphere of the brain or the thigh muscle, and potential target organs were collected for qPCR analysis.

Administration of NR1 at doses up to 2×10⁶ NR1 cells IC or 1×10′ cells IM was not associated with any unexpected mortality, clinical findings, changes in body weight or food consumption, changes in clinical pathology or bone marrow endpoints, or macroscopic or microscopic observations. There were no NR1-related early deaths and/or euthanasia and no teratoma formation was seen in the brain or in peripheral tissues in any group treated with NR1 alone. NR1 was well tolerated at the high dose, 2×10⁶ cells/animal, for the study duration of 180 days.

HuNu staining for NR1 was performed on the injection track of the right hemisphere of the brain in Groups 1 through 10 and the IM injection site from animals in Groups 11 through 13. In Groups 2 and 3, which received IC injections of NR1 drug product only, NR1 cells were present at minimal to moderate levels in low-dose (17/20 animals) and high-dose (16/20 animals) at 28 days post-dose. At the Day 180 necropsy, NR1 cells were present at minimal to marked levels in the low-dose (22/30 animals) and high-dose (23/30 animals) animals. At both Days 28 and 180 post-transplant NR1 cells were mostly present at the injection sites, visualized microscopically as cavitated spaces microscopically, within the brain parenchyma or less frequently within the ventricular spaces, and there was no evidence of migration of NR1 cells away from the injection sites. A few animals exhibited mild cartilaginous/osseous metaplasia in the meninges and/or the injection track, which was attributed to residual debris from the craniotomy, a previously observed artifact of this dosing procedure in rats. In a single male (Animal No. 3018) the metaplasia (described as a small encapsulated focus of well-differentiated, non-neoplastic mesenchymal metaplasia characterized by an island of cartilage, minimal bone, and a thick capsule of dense fibrous connective tissue) had scattered HuNu-positive cells, attributed to the deposition of a portion of the NR1 cell medium during the dosing procedure.

The athymic rat model, a predicted in earlier studies and confirmed in this GLP study, was able to detect the formation of stem cell-related teratomas, showing its suitability for the toxicity and teratoma/carcinogenic assessment of NR1. When animals received a combination of NR1 cells spiked with H9 cells, macroscopically visible teratomas were evident at the brain injection site, resulting in the early termination of 9/20 animals in Group 4 (50% H9), 7/20 animals in Group 5 (25% H9), and 2/10 animals in Group 6 (10% H9). No macroscopic teratomas occurred in Groups 7 (1% H9) and 8 (0.1% H9). There were no early termination deaths in groups that received less than a 10% spiked fraction of H9 cells. Complete data are presented in Table 2.4.4.1.A: Incidence of Teratoma Formation. As expected, teratomas were visible earlier in groups that dose solutions spiked with higher concentrations of H9 cells. Microscopic examinations were consistent, with dose-dependent occurrence of teratomas in all H-9-spiked treatment groups. Microscopically the teratomas present in Groups 4-6 consisted of tissues from the three germ layers endoderm, mesoderm and ectoderm, including a mixture of tissues such as skin, glands, cartilage and/or bone formation, as well as nervous tissue. These teratomas tended to form large masses. Comparatively, the teratomas in animals in Group 7 (NR1 spiked with 1% H9; 4/20 animals) and Group 8 (NR1 spiked with 0.1% 79; 1/19 animals) were smaller and consisted primarily of small formations of haphazard-appearing nervous tissue with choroid plexus-like formation, ependymal epithelium, and/or bulging areas of near-normal looking white matter and did not contain tissues derived from all three germ layers as is considered characteristic of teratomas. All teratomas seen macroscopically and/or microscopically in the H1-9 spiked treatment groups stained positive for HuNu. No evidence of teratoma formation outside of the brain or distant from the injection site was observed in any H-9-spiked or any of the NR1 drug product-only treated groups.

TABLE 3
Incidence of Teratoma Formation

| Group | | Cell Dose/Animal^{a} | Dose | Teratoma | Teratoma-Related |
|---|---|---|---|---|---|
| No. | Treatment^{a} | (% H9 hESC) | Route | Incidence (%) | Early Termination (%) |
| 1 | Cell Medium | 0 | IC | 0/20 (0) | 0/20 (0) |
| 2 | NR1 − Low | NR1: 4 × 10⁵ | IC | 0/50 (0) | 0/50 (0) |
| 3 | NR1 − High | NR1: 2 × 10⁶ | IC | 0/50 (0) | 0/50 (0) |
| 4 | NR1 + H9 | NR1: 1 × 10⁶ | IC | 12/18 (67) | 10/20 (50) |
| | | H9: 1 × 10⁶ (50%) | | | |
| 5 | NR1 + H9 | NR1: 1.5 × 10⁶ | IC | 9/20 (45) | 7/20 (35) |
| | | H9: 5 × 10⁵ (25%) | | | |
| 6 | NR1 + H9 | NR1: 1.8 × 10⁶ | IC | 5/20 (25) | 2/20 (10) |
| | | H9: 2 × 10⁵ (10%) | | | |
| 7 | NR1 + H9 | NR1: 1.98 × 10⁶ | IC | 4/20 (20) | 0/20 (0) |
| | | H9: 2 × 10⁴ (1%) | | | |
| 8 | NR1 + H9 | NR1: 2 × 10⁶ | IC | 1/19 (5.3) | 0/19 (0) |
| | | H9: 2 × 10³ (0.1%) | | | |
| 9 | H9 | H9: 1 × 10⁶ | IC | 0/10 (0) | 0/10 (0) |
| 10 | H9 | H9: 2 × 10³ | IC | 0/10 (0) | 0/10 (0) |
| 11 | NR1 | NR1: 1 × 10⁷ | IM | 0/40 (0) | 0/40 (0) |
| 12 | NR1 + H9 | NR1: 5 × 10⁶ | IM | 0/20 (0) | 0/20 (0) |
| | | H9: 5 × 10⁶ (50%) | | | |
| 13 | NR1 + H9 | NR1: 1 × 10⁷ | IM | 0/20 (0) | 0/20 (0) |
| | | H9: 1 × 10⁴ (0.1%) | | | |

^{b}Number of animals affected per total number of treated animals

No human cells or teratomas were detected when H9 cells alone were administered IC into the brain or NR1 cells or when NR1 cells spiked with H9 cells were injected IM peripherally during post-treatment observation as long as 180 days after transplantation. In the H9-treated groups, cavitation was noted at the site of administration in the brain, suggesting the cells did not survive or engraft. The reason for the lack of growth of H9 cells in the brain when administered alone at a dose of 1×10⁶ is not clear, but clearly H9 engraftment was not supported. It was not due to lack of viability of the H9 cell lot used, however, since an equal number of H9 cells spiked into 1×10⁶ NR1 cells resulted in the formation of teratomas (Group 4), as did the lower numbers of H9 cells used in the Groups 5-8. Rats that received IC injections of 1×10⁶ H9 cells/animal in a different cell medium, DMEM/F12 plus 0.5% HSA, with 30% rather than 40% Matrigel, developed visible teratomas by approximately 50 days post-treatment. The lack of growth of the IM-injected cells (NR1, H9 and NR1+H9) at doses up to 1×10¹ was attributed to a less permissive growth environment at the IM injection site.

The results of this study demonstrated that administration of the cGMP NR1 drug product at a dose up to 2×10⁶ cells into the parenchyma of the brain via IC injection were well tolerated and resulted in no animal deaths or evidence of local or systemic toxicity, tumorigenicity or teratoma formation NR1 engrafted and persisted for the duration of the study and remained at the site of administration NR1, the formulated drug product, did not form teratomas at doses up to 2×10⁶ NR1 cells/animal in the brain or any peripheral tissue over a 180-day treatment-free observation period even in the presence of growth-promoting Matrigel the athymic nude rat model used was able to detect the development of teratomas when H9 cells were spiked in NR1 at specified concentrations and injected IC at doses as low as 2000 H9 cells (0.1% of the NR1 dose).

Treatment with doses up to 2×10⁶ cells/animal of cGMP NR1 drug product alone (which has less than 0.01% residual H9 cells) did not result in the formation of teratomas over the 6-month duration of the study even when transplanted in a cell medium containing 40% Matrigel, which was intended to maximize the likelihood of cell growth. Matrigel is a complex protein mixture derived from mouse Engelbreth-Holm-Swarm sarcoma cells that is reported to contain murine growth factors. Matrigel was only used to promote survival of the human cells in rodents and will not be included in the clinical NR1 drug product injection medium.

### Overview and Conclusions

As summarized above, a number of pharmacology, biodistribution and toxicology studies were conducted with NR1 drug product. The key to conducting relevant studies is using relevant animal models. The model(s) used must support the survival of human NR1 cells in the rodent at sufficient number and for a sufficient duration of time to assess efficacy, distribution and toxicity. Immunodeficient or immunosuppressed rodent models are used to minimize the potential for rejection of the human NR1 drug product in the rodent test systems. Specifically, for pharmacology studies, to best judge efficacy the animal model and experimental conditions should replicate the clinical conditions of the human population as closely as possible, including the mode and route of treatment, the target for NR1 activity and the measurement of outcome using methods and endpoints relevant to human subjects. For biodistribution studies, the model should also mimic the clinical conditions, including an ischemic subcortical stroke, of the human subjects as closely as possible, i.e., stroked animals should be used, to deliver a pertinent assessment of NR1 distribution in the stroked brain. For toxicology studies, the animal model should be able to assess the general toxicity of NR1 and, in the case of this stem cell-derived cell therapy, be able an animal model that support the growth and development of stem cell-related teratomas and tumors. To avoid possible confounding effects of stroke on evaluation of toxicity, tumorigenicity and teratoma formation, the toxicology model should be in naïve, non-stroked animals.

### Animal Models

Pharmacology: the primary model used was a middle cerebral artery occlusion model of ischemic subcortical stroke (ISS) in immunosuppressed Sprague-Dawley rats. This is a well-characterized nonclinical model that is routinely used in the stroke field and provides the closest match to the proposed NR1 target clinical indication of patients with chronic ISS. This middle cerebral artery occlusion model of ISS in immunosuppressed Sprague-Dawley rats was used to evaluate the effect of treatment with NR1 in acute (NR1 treatment at 7 days post-stroke) and chronic settings (NR1 treatment at 28 days post-stroke), with a total study duration of 3 months. A supportive efficacy study was conducted in another clinically relevant animal model, the immunodeficient athymic nude rat that received an ischemic cortical stroke (ICS) via occlusion of the distal middle cerebral artery

Biodistribution: the GLP biodistribution study was also conducted in the ISS model in immunosuppressed Sprague-Dawley rats to provide the most complete assessment of the persistence and migration of IC-injected NR1 cells in a stroked animal, best modeling the clinical condition of human subjects with chronic stroke

Toxicology: the GLP toxicology study was conducted in immunodeficient athymic nude rats in the presence of Matrigel to maximize the likelihood of NR1 survival and growth and the development of teratomas. Matrigel is a complex protein mixture derived from mouse Engelbreth-Holm-Swarm sarcoma cells that is reported to contain murine growth factors (Hughes et al., 2010). Treatment groups that received H9 hESC were included to confirm that the athymic rat model could support the development of teratomas and had a level of sensitivity sufficient to detect very low levels of contaminating hESC. Additionally, the study was of sufficient duration (180 days) to allow time for teratoma development should any occur.

Survival (Persistence) of NR1 Cells in Animal Models. Persistence of NR1 cells in the models is critical to obtaining accurate predictions of human activity, distribution and toxicity. The following approaches were taken to improve NR1 cell survival and persistence:

In the first step in selecting the correct animal model, NR1 survival was compared in different immunodeficient rodent models. The studies compared adult and neonatal athymic nude rats and adult NSG mice. The highest rate of cell survival and persistence was measured in immunodeficient adult athymic nude rats, which were used to further test approaches to improve persistence.

Next, specific immune cell populations were depleted in adult athymic nude rats to try to improve human-origin NR1 cell survival. The number of microglial cells, which act as macrophages in the CNS, was depleted by 40-50% in pilot studies, but such decrease did not increase NR1 persistence. Similarly, depletion of the number of Natural Killer cells by up to 80-90% had no effect on NR1 cell persistence. Neither approach was used in subsequent studies.

Last, the effect of including Matrigel in the cell injection medium was evaluated. Pilot studies showed that Matrigel significantly improved long-term NR1 cell survival in the adult athymic nude rat. Matrigel was therefore included in the injection medium in the toxicology studies to improve NR1 survival and growth. It was not used in the pharmacology and distribution studies because of possible confounding effects.

Cell persistence was measured in the animal models in the pivotal GLP biodistribution and toxicology studies to confirm the presence of NR1. In the pharmacology studies, there was no evidence for the presence of NR1 cells in the brain, despite the positive effects of NR1 treatment. However, in the GLP biodistribution study in the ISS stroke model, the same model used in the primary pharmacology study, persistence of low numbers of NR1 cells for as long as 45 days post-dose was detected using IHC and qPCR methods. The difference in findings in the studies is probably related to better sample preservation and more sensitive detection methods used in the biodistribution study compared to the pharmacology study, in which tissue samples to measure cell persistence were collected only after the critical tissue for other endpoints had been harvested. The results of the biodistribution study accurately demonstrate the long-term persistence of NR1 cells in the test system. Cell persistence in the adult athymic nude rat in the presence of Matrigel was shown in several pilot studies and in the GLP toxicology study. IHC and qPCR assays in the latter study demonstrated the persistence of NR1 cells in naïve athymic nude rats for as long as 180 days post-treatment in the presence of Matrigel.

In conclusion, although persistence study results, while variable, provide weight of evidence showing that NR1 cells persist for several months after IC injection, and as long as 6 months in the athymic nude rat model.

Detection of Teratomas. To provide a relevant assessment of the potential adverse effects of the NR1 drug product the toxicology model must be able to support the development of stem cell-derived teratomas. To confirm that the adult athymic nude rat model that included Matrigel could detect teratomas, selected treatment groups in the GLP toxicology study received IC injections of mixtures of NR1 cells spiked with pre-specified levels of H9 hESC that comprised from 0.1% to 50% of the total NR1 cell dose. No teratomas were detected at 28 days after transplantation, but by 180 days post treatment, a dose-related incidence of teratomas affecting up to two-thirds of treated animals at the 50% H9-spike dose was seen, confirming that the athymic nude rat model met the requirement of supporting the development of teratomas.

Measurement of the Toxicity of NR1. Results from pilot toxicology studies in several immunocompromised laboratory rodent models guided the selection of the athymic nude rat model as the optimal model for the GLP toxicology assessment by demonstrating the prolonged NR1 cell survival required to fully assess toxicity, including the formation of teratomas. The adult athymic nude rat model that included Matrigel in the cell injection medium showed enhanced NR1 survival and persistence. The key endpoints in this study were the assessment of the general toxicity of NR1, confirmation of NR1 persistence in the brain, demonstration of its lack of migration from the treatment site and evaluation of its potential to cause teratomas as well as other toxicology endpoints.

General toxicity. NR1 at doses up to 2×10⁶ NR1 cells IC or 1×10⁷ cells IM was well tolerated. There was no unexpected mortality, clinical findings, changes in body weight or food consumption, changes in clinical pathology or bone marrow endpoints, or in macroscopic and microscopic observations. There were no NR1-related early deaths and NR1 was well tolerated for as long as 180 days post-dose.

Cell persistence and migration. NR1 did not distribute or migrate from the intracerebral site of administration to other areas of the brain or to peripheral tissues. Sensitive IHC and qPCR assessments confirmed long-term survival of low numbers of NR1 cells for as long as the 180-day duration of the study.

Teratoma formation. No teratomas were detected in the brain or in peripheral tissues in any rat treated with NR1 drug product only. A total of 100 athymic nude rats received up to 2×10⁶ NR1 cells per animal and were followed for up to 6 months post-transplantation. The model was able to detect teratomas at H9 spike levels as low 0.1% H9 cells (2000 H9 cells added to a dose of 2×10⁶ NR1 cells/animal) in prepared mixtures of NR1 and H9 cells. NR1 drug product has been shown to contain <0.01% H9 cells.

The results of this study showed that NR1 drug product was well tolerated at the high dose, did not migrate from the site of intracerebral administration, persisted at the injection site for the duration of the study and did not cause the development of teratomas for up to 180 days post-dose.

### Measurement of Efficacy and Mechanism of Action

Efficacy Measurements. NR1 treatment resulted in the partial recovery of sensorimotor function in stroked immunosuppressed SD rats, as measured by multiple assessments and reflected in positive outcomes in well-known measurement instruments:

The primary demonstration of efficacy was conducted in cyclosporine-immunosuppressed Sprague-Dawley (SD) rats using a middle cerebral artery occlusion model of ISS (USF-1). Here, IC transplantation of NR1 promoted recovery of general motor behavior, neurological function and motor coordination. The onset of functional recovery across tests occurred at month 2 post-transplantation and persisted up to at least 3 months post-transplantation.

Supportive study results were seen in an ICS model (Stanford-5) in athymic nude rats, which had similar results: IC transplantation of NR1 cells resulted in improved sensorimotor function, as assessed by several performance measurement instruments. The improvements, first seen at 4 weeks post-treatment, persisted until animal termination 8 weeks post-dose.

Mechanism of Action. The studies provide evidence suggesting that the activity of NR1 cells is related in part to an immunomodulatory function and to NR1-related stimulation of secretion of active modulatory factors, as follows: Histological assessments the pharmacology studies suggesting a link between decreased peri-infarct microglial/immune cell activation and NR1-associated enhancement of functional recovery.

Evidence indicating that NR1-related polarization of macrophages from an M1/pro-inflammatory state to an M2/anti-inflammatory state, primarily by secreting factors that push both unstimulated and M1-/M2-stimulated macrophages toward the beneficial M2/anti-inflammatory state.

IHC analysis demonstrating qualitatively that NR1-treated stroked rats have more activated macrophages/microglia and vehicle-treated animals than do control rats

TSPO-PET imaging in stroked rats showing increased immune cell activity in NR1-treated compared to vehicle-treated rats, both near the transplantation site and at more distant but connected brain regions, implying widespread immunomodulatory effects of the NR1.

Secretome TRAP analysis identifying 175 highly expressed genes encoding for secreted proteins, with the top 20 enriched processes associated with brain repair, including extracellular matrix (ECM) remodeling (involved in most brain repair mechanisms) and other processes related to inflammation and axon guidance. TRAP/RNAseq analysis of cultured NR1 identified over 400 genes that encode for secreted factors with biological processes central to NR1-associated stroke recovery: inflammation (162 genes, of which 144 genes code for proteins associated with cytokine activity) and ECM organization (65 genes). Of the genes of interest identified in vivo, NRG3 and TGFb3 were upregulated in response to the stroke environment

Brain plasticity studies showing that NR1 transplantation significantly increased motor circuit activity in peri-infarct cortex layers of live brain slices at Day 7 after transplantation. Brains from animals treated with NR1 cells differentially expressed nearly 300 genes known to stimulate adult neural precursor cell proliferation, neuronal differentiation, gated channels directly involved in the electrophysiological properties of neurons, axon guidance, axonogenosis, and synaptogenesis.

The investigational studies strongly implicate immunomodulatory effects via secretion by NR1 cells of modulating factors that contribute to the plasticity of peri-infarct regions in the mechanism of action of NR1. Evidence suggests that NR1 has this effect due to the stimulation of processes believed central to stroke recovery, including inflammation and the organization of the extracellular matrix, via in part the increased expression of genes known to affect immune function and stimulate brain plasticity in vivo.

### Biodistribution

The GLP biodistribution study was conducted in immunosuppressed Sprague-Dawley rats using the middle cerebral artery occlusion model of ISS to best model the clinical conditions in human subjects with chronic stroke. Rats were treated with NR1 duplicating the treatment dose and time points used in the primary efficacy study. NR1 cells were quantified in the brain and neural axis, peripheral tissues and gross lesions using a sensitive validated real time qPCR assay for NR1 gDNA and IHC staining of tissues with the human anti-nuclear antibody HuNu. NR1 cells at doses of up to 4×10⁵ cells/animal persisted within the brain for at least 45 days in acute and chronic stroke animals after transplantation into the cortex and striatum of the brain. NR1 cells were not detected outside the region of the site of administration, with no migration to adjacent areas of the brain or to peripheral tissues.

### Conclusions

NR1 treatment in the ischemic subcortical stroke (ISS) model in immunosuppressed Sprague-Dawley rats promotes the partial recovery of sensorimotor function as reflected in positive outcomes in several well-known measurement instruments. The exact mechanisms of the recovery are not yet fully understood but appear to be related at least in part to an immunomodulatory function including treatment-related decreases in immune cell activation that correlate with the observed partial and potentially full functional recovery after NR1 treatment. Studies suggest that NR1 acts via the stimulation of processes critical to stroke recovery, including inflammation and the organization of the extracellular matrix, via the secretion of immunomodulatory factors in vivo. NR1 cell persistence studies showed that NR1 cells are present in the brain for a prolonged period after intracerebral (IC) injection, correlating with the improvements in sensorimotor function seen in the pharmacology studies, but that there was no migration of NR1 cells away from the site of the intracerebral injection. Toxicology studies established that IC transplantation of the NR1 drug product (that has been show to contain less than 0.01% H9 cells) did not result in animal deaths, that there was no migration of cells away from the treatment site and that after injection of NR1 cells alone did not result in general toxicity or teratoma formation for as long as 6 months after injection after treatment with NR1 cells alone, even when growth-promoting Matrigel was included in the injection medium.

In summary, NR1 drug product was well tolerated at doses up to 2×10⁶ cells/animal, which extrapolates to 148×10⁶ cells per dose in human subjects, did not migrate from the site of intracerebral administration, persisted at the injection site for several months, was well tolerated and did not cause the development of teratomas or tumors.

### Example 3

### Neural Transplantation of NR1 Neural Progenitor Cells (NPCs) in an Ischemic Stroke Model

### Animal Requirements

Species: Rat Age/Weight: 2 months old/250-300 g Sex: male
Strain: Sprague-Dawley Rats
Number of Animals: 95 adult male rats enrolled in the study

Immunosuppression Regimen: Rats were immunosuppressed 2 days before cell transplantation and daily thereafter for one week with i.p. injections of cyclosporine A (20 mg/ml, Sandimmune, Novartis Pharmaceuticals, diluted in sterile, normal saline). Thereafter oral cyclosporine was used at 210 mg/ml in drinking water (sterile de-ionized water) until euthanasia.

This study was designed to evaluate potential therapeutic value of intracerebral transplantation of NR1 NPCs (supplied by Stanford University) in an animal model of adult ischemic stroke. Transplantation with two dose levels of cells was carried out at either 7 or 28 days after stroke with functional readouts of behavioral and histological deficits conducted during the subsequent 3-month period post-grafting. We characterized locomotor and neurological performance at baseline (prior to stroke), then at either 7 days or 28 days after stroke (prior to transplantation) and again at monthly intervals post-grafting. Following completion of behavioral testing at 3 months post-grafting, animals were euthanized by transcardial perfusion and brains harvested and shipped to Stanford University for histological examination (see scanned Logbook, pages 57-61). The primary endpoint was that intracerebral transplantation of NR1 NPCs at either 7 days or 28 days after stroke improved behavioral recovery as seen in a statistical improvement in the elevated body swing test (EBST). The secondary endpoint was that intracerebral transplantation of NR1 NPCs at either 7 days or 28 days after stroke improved behavioral recovery as seen in a statistical improvement in the neurological exam and rotorod tests.

### Test and Control Articles

Test Article. NR1 NPCs, passage 18 cells and manufactured under GMP at City of Hope (Batch No. 1299-128-0002).
Control (Vehicle). Plasmalyte-A (Baxter cat #2B2544, lot #C878264) containing 0.5% human serum albumin (HSA) (Talecris Biotherapeutics, Inc. cat #13533-684-20, lot #26NJ6H1).

### Experimental Design

1. Treatment conditions are shown in Table 4.

### Treatment Conditions

| Group | Group Size | Test Article | Dose (dose volume) | Transplant Time |
|---|---|---|---|---|
| A | 15 | NR1 NPCs Low Dose | 1e5 cells (9 μL) | 7 days post-stroke |
| B | 15 | NR1 NPCs High Dose | 4e5 cells (9 μL) | 7 days post-stroke |
| C | 15 | Vehicle | 9 μL | 7 days post-stroke |
| D | 15 | NR1 NPCs Low Dose | 1e5 cells (9 μL) | 28 days post-stroke |
| E | 15 | NR1 NPCs High Dose | 4e5 cells (9 μL) | 28 days post-stroke |
| F | 15 | Vehicle | 9 μL | 28 days post-stroke |

Total of 100 animals (90 study animals plus 10 spares) received MCAo stroke. 95 animals were enrolled for the in-life study; All stroke animals were randomly assigned to Groups A-F and treated on Day 7 or Day 28 post-stroke. General health of the animals were monitored by weekly clinical observations and recording of body weights. Animals were euthanized at 3 months post-grafting by transcardial perfusion with 4% paraformaldehyde.

Outcome Measures: Transplant outcome was evaluated using the following parameters: 1) locomotor behavior via elevated body swing test (EBST), rotorod, and neurological performance via neurological examination. These behavioral tests were conducted in USF; 2) infarct volume via Nissl/H&E histologic stains; 3) graft survival via immunohistochemistry using specific antibody shown to detect human cells, and; 4) preliminary mechanism-based immunohistochemical analyses of neovascularization, altered inflammatory response and host cell survival in the peri-infarct area. For outcome assays #2, #3, and #4, these are to be conducted in Stanford University.

### Parameters of Cell Transplant Outcomes

| Test | Objective |
|---|---|
| *EBST | Reveals locomotor performance after stroke and recovery after transplantation |
| *Rotorod | Reveals motor coordination after stroke and recovery after transplantation |
| *Neurological Exam | Reveals motor coordination after stroke and recovery after transplantation |
| **H&E/Nissl histology | Reveals extent of cerebral ischemic cell loss |
| **HuNu | Reveals survival of grafted human stem cells |
| **IBA1 | Reveals macrophage/microglial cells |
| **RECA-1 | Reveals blood vessels |

### Experimental Procedures

MCAo stroke surgery: All surgical procedures were conducted under aseptic conditions. The animals will be anesthetized with 1.5% isofluorane and checked for pain reflexes. Under deep anesthesia, animals underwent the MCAo surgery. The MCA suture technique involved insertion of a filament through the carotid artery to reach the junction of the MCA, thus blocking the blood flow from the common carotid artery, as well as from the circle of Willis. The right common carotid artery was identified and isolated through a ventral midline cervical incision. The suture size was 4-0, made of sterile, non-absorbable suture (Ethicon, Inc.), with the diameter of the suture tip tapered to 24 to 26-gauge size using a rubber cement. About 15 to 17 mm of the filament was inserted from the junction of the external and internal carotid arteries to block the MCA. The right MCA was occluded for one hour. Based on our studies and several others, a one-hour occlusion of the MCA results in maximal infarction. In addition, the length and size of the tip of the suture have been found to produce complete MCA occlusion in animals weighing between 250 to 350 g. A heating pad and a rectal thermometer allowed maintenance of body temperature within normal limits. To determine successful occlusion and reperfusion, a laser Doppler was used. The laser Doppler probe measured cerebral blood flow before, during and after occlusion. Only animals that displayed a reduction of >80% cerebral blood flow during the MCA occlusion period as revealed by laser Doppler were included in the subsequent procedures (i.e., transplantation). All other animals were immediately excluded from the study. In addition, animals were excluded (i.e., euthanized) for humane reasons based on health observations and body weights (n=8 died during stroke surgery). Finally, animals were randomly assigned to the treatment groups with sampling for the treatment groups as follows: at least 15 animals each surgical day exhibited successful MCA occlusion (>80% cerebral blood flow); at least 15 animals were transplanted on the pre-set time point post-stroke (day 7 or day 28); on each transplant day, 5 animals were randomly assigned to NR1 NPCs high dose, 5 animals randomly assigned to NR1 NPCs low dose, and 5 animals randomly assigned to vehicle. Rats in all groups were immunosuppressed 2 days before cell or vehicle transplantation and daily thereafter for one week with i.p. injections of cyclosporine A (20 mg/ml, Sandimmune, Novartis Pharmaceuticals). Thereafter oral cyclosporine was used at 210 mg/ml in drinking water until euthanasia.

Animals were observed weekly for general health during the course of the study. Body weights were recorded at the initiation of treatment, weekly during the study and at the termination of the study.

Grafting Procedures: Fifteen animals were transplanted per day. All surgical procedures were conducted under aseptic conditions. Animals were anesthetized with 1.5% isofluorane. Once deep anesthesia was achieved (by checking for pain reflexes), hair was shaved around the area of surgical incision (skull area) with enough border to prevent contaminating the operative site, followed by two surgical germicidal scrubs of site, and draping with sterile drapes. The animal was then fixed to a stereotaxic apparatus (Kopf Instruments). A 26-gauge Hamilton 701 microsyringe needle was then lowered into a small burred skull opening (transplant coordinates were adjusted to correspond with the striatum: 0.5 mm anterior and 2.8 mm lateral to bregma, and 5.0 mm, 4.0 mm, and 3.0 mm below the dural surface; based on the atlas of Paxinos and Watson, 1998). Within this single needle pass, 3 deposits of the test articles including 2 into the striatum and 1 into the cortex were made (as noted above: 5.0 mm, 4.0 mm, and 3.0 mm below the dural surface). The target areas were the medial striatum and medial cortex which correspond to the ischemic peri-infarct (or penumbra) area, based on previously established target sites for similar stereotaxic implants (Yasuhara et al., Stem Cells and Dev, 2009). Each deposit consisted of 3 ul volume (e.g., a dose of 400,000 cells was injected in a total volume of 9 ul) infused over a period of 3 minutes. Following an additional 2-minute absorption time, the needle was retracted and the wound closed stainless steel wound clip. A heating pad and a rectal thermometer allowed maintenance of body temperature at about 37° C. throughout surgery and following recovery from anesthesia.

Behavioral and neurological tests: All investigators testing the animals were blinded to the treatment condition. Animals were subjected to elevated body swing test (EBST), neurological exam and rotorod. These tests have been shown to be sensitive assays of motor/sensory deficits produced by unilateral stroke surgery. EBST involved handling the animal by its tail and recording the direction of the swings. The test apparatus consisted of a clear Plexiglas box (40×40×35.5 cm). The animal was gently picked up at the base of the tail, and elevated by the tail until the animal's nose is at a height of 2 inches (5 cm) above the surface. The direction of the swing, either left or right, was counted once the animals head moved sideways approximately 10 degrees from the midline position of the body. After a single swing, the animal was placed back in the Plexiglas box and allowed to move freely for 30 seconds prior to retesting. These steps were repeated 20 times for each animal. Normally, intact rats display a 50% swing bias, that is, the same number of swings to the left and to the right. A 75% swing bias would indicate 15 swings in one direction and 5 in the other during 20 trials. We have previously utilized the EBST, and noted that lesioned animals display >75% biased swing activity at one month after a nigrostriatal lesion; asymmetry is stable for up to six months. About one hour after the EBST, Neurological exam was conducted following the procedures previously described with minor modifications. Neurologic score for each rat was obtained using 3 tests which include (1) forelimb retraction, which measures the ability of the animal to replace the forelimb after it is displaced laterally by 2 to 3 cm, graded from 0 (immediate replacement) to 3 (replacement after several seconds or no replacement); (2) beam walking ability, graded 0 for a rat that readily traverses a 2.4-cm-wide, 80-cm-long beam to 3 for a rat unable to stay on the beam for 10 seconds; and (3) bilateral forepaw grasp, which measures the ability to hold onto a 2-mm-diameter steel rod, graded 0 for a rat with normal forepaw grasping behavior to 3 for a rat unable to grasp with the forepaws. The scores from all 3 tests, which were done over a period of about 15 minutes on each assessment day, were added to give a mean neurologic deficit score (maximum possible score, 9 points divided by 3 tests=3). Following the neurological tests, animals were tested in Rotorod, which involved placing the rats on the rotorod cylinder. The time the animals remained on the rotorod was measured. The speed slowly increased from 4 to 40 rpm within a period of 60 seconds. The trial ended when the animal fell off the rungs or gripped the device and spun around for 2 consecutive revolutions. The animals were trained for 3 days before stroke. The maximum duration (in seconds) on the device was recorded with 3 rotorod measurements 1 day before stroke induction (baseline). Animals were subjected to all 3 tests at baseline (prior to stroke), then at 7 and 28 days after stroke (prior to transplantation) and at monthly intervals over the subsequent 3 months post-grafting.

Histology: Following the 3 months post-transplantation period, animals were euthanized and brains harvested and shipped to Stanford University for histological examination.

Statistical Analyses: Upon study completion, the mean behavioral data were calculated and compared with other treatment groups. Statistical analysis were performed to compare treatment groups to control (vehicle administration) using multiple comparison ANOVA and Bonferonni post-hoc analyses.

### Results

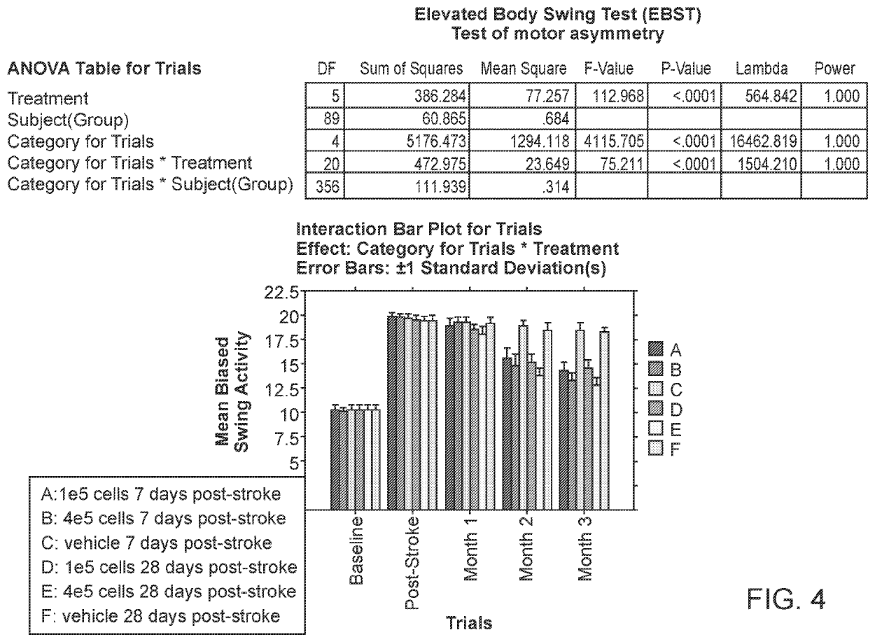

Shown in FIG. 4, intracerebral transplantation of NR1 NPCs ameliorates motor dysfunction as shown by EBST. ANOVA revealed that main effects of treatment (p<0.0001) and trials (p<0.0001), as well as interaction effects (p<0.0001) were significant. Within group comparisons showed that stroke animals that received vehicle alone (C,F) displayed significant motor deficit post-stroke and persisted throughout the 3-month study period (p's<0.005 versus baseline). In contrast, those stroke animals that received NR1 NPCs (A, B, D, E) also exhibited motor impairment at post-stroke and month 1 post-transplantation (p's<0.005 versus baseline), but thereafter demonstrated recovery of motor function starting at month 2 and month 3 post-transplantation (p's<0.005 versus post-stroke). These transplanted stroke animals, while recovered at month 2 and month 3 post-transplantation, remained impaired compared to baseline (p's<0.005). Between group comparisons revealed that all groups displayed comparable motor function at baseline, and also exhibited similar motor impairments at post-stroke and month 1 post-transplantation. However, at month 2 and month 3 post-transplantation, those stroke animals that received NR1 NPCs (A, B, D, E) exhibited significant improvements in motor behavior compared to vehicle-infused stroke animals (C,F) (p's<0.0033). Generally, while both transplantation at acute and chronic phase of stroke and both low and high cell doses significantly promoted recovery of motor function, transplantation at the chronic phase and at the high dose appeared to be the most optimal transplant regimen (see comparisons between E versus A, B, or D, p's<0.0033).

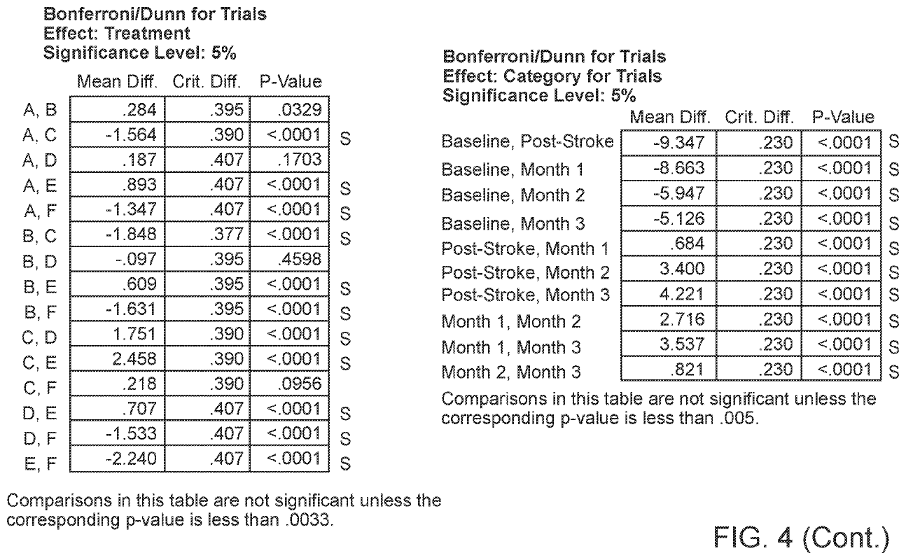

FIG. 5. Intracerebral transplantation of NR1 NPCs NSCs attenuates neurological impairment as shown by neurological test. ANOVA revealed that main effects of treatment (p<0.0001) and trials (p<0.0001), as well as interaction effects (p<0.0001) were significant. Within group comparisons showed that stroke animals that received vehicle alone (C,F) displayed significant neurological deficit post-stroke and persisted throughout the 3-month study period (p's<0.005 versus baseline). In contrast, those stroke animals that received NR1 NPCs (A, B, D, E) also exhibited neurological dysfunction at post-stroke and month 1 post-transplantation (p's<0.005 versus baseline), but thereafter demonstrated recovery of neurological function starting at month 2 and month 3 post-transplantation (p's<0.005 versus post-stroke). These transplanted stroke animals, while recovered at month 2 and month 3 post-transplantation, remained impaired compared to baseline (p's<0.005). Between group comparisons revealed that all groups displayed comparable neurological function at baseline, and also exhibited similar neurological impairments at post-stroke and month 1 post-transplantation. However, at month 2 and month 3 post-transplantation, those stroke animals that received NR1 NPCs (A, B, D, E) exhibited significant improvements in neurological performance compared to vehicle-infused stroke animals (C,F) (p's<0.0033). Both transplantation at acute and chronic phase of stroke and both low and high cell doses did not significantly differ among themselves in improving the neurological outcomes (p's>0.0033).

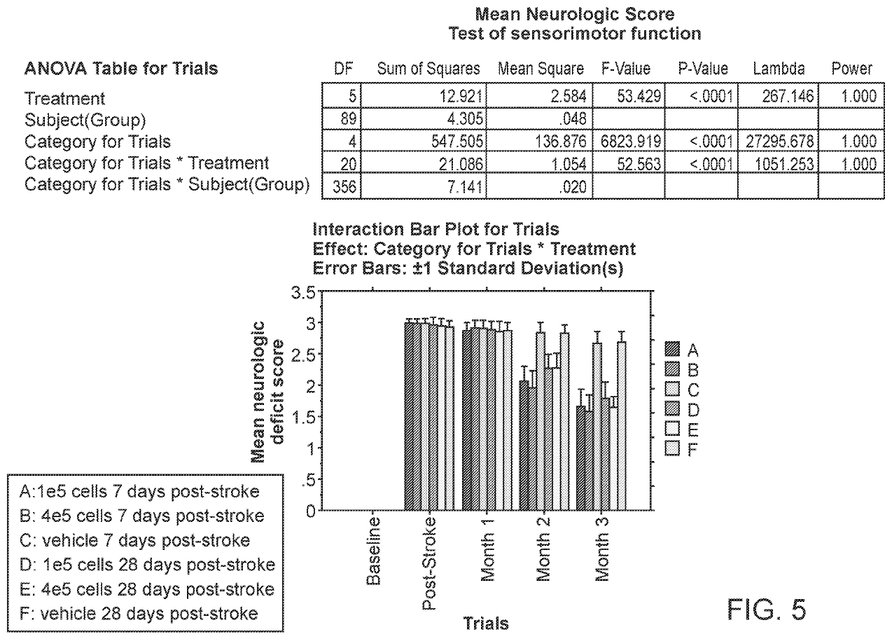

FIG. 6: Intracerebral transplantation of NR1 NPCs promotes recovery of motor coordination as shown by Rotorod test. ANOVA revealed that main effects of treatment (p<0.0001) and trials (p<0.0001), as well as interaction effects (p<0.0001) were significant. Within group comparisons showed that stroke animals that received vehicle alone (C,F) displayed significant deficit in motor coordination post-stroke and persisted throughout the 3-month study period (p's<0.005 versus baseline). In contrast, those stroke animals that received NR1 NPCs (A, B, D, E) also exhibited impaired motor coordination at post-stroke and month 1 post-transplantation (p's<0.005 versus baseline), but thereafter demonstrated recovery of motor coordination starting at month 2 and month 3 post-transplantation (p's<0.005 versus post-stroke). These transplanted stroke animals, while recovered at month 2 and month 3 post-transplantation, remained impaired compared to baseline (p's<0.005). Between group comparisons revealed that all groups displayed comparable motor coordination at baseline, and also exhibited similar impairments in motor coordination at post-stroke and month 1 post-transplantation. However, at month 2 and month 3 post-transplantation, those stroke animals that received NR1 NPCs (A, B, D, E) exhibited significant improvements in motor coordination compared to vehicle-infused stroke animals (C,F) (p's<0.0033). Generally, while both transplantation at acute and chronic phase of stroke and both low and high cell doses significantly promoted recovery motor coordination, transplantation at the chronic phase and at the high dose appeared to be the most optimal transplant regimen (see comparisons between E versus A, B, or D, p's<0.0033).

The present data revealed that intracerebral transplantation of NR1 NPCs promotes recovery of general motor behavior, neurological function and motor coordination as shown by EBST, neurological test, and Rotorod test, respectively. The onset of functional recovery across of tests was at month 2 post-transplantation, and persisted up to at least month 3 post-transplantation (end of study). Overall, both transplantation at acute and chronic phase of stroke and both low and high cell doses significantly promoted functional recovery, but transplantation at the chronic phase and at the high dose appeared to be the most optimal transplant regimen based on EBST and Rotorod test. There were no observable overt side effects, with weights and general health conditions comparable between stroke animals that received the transplants and those stroke animals that received the vehicle. In summary, the findings from this study support the use of NR1 NPCs as donor cells for intracerebral transplantation in stroke.

### Example 4

### NR1 Human Neural Progenitor Cell Dose-Response Study of Efficacy in the Nude Rat Cortical Stroke Model

The purpose of this non-GLP study was to test the efficacy of NR1 human neural progenitor cells (hNPC) to increase functional recovery in an athymic nude (NIH) rat cortical model of ischemic stroke. Previous studies by our group have demonstrated that direct intraparenchymal transplantation of NR1 or other hNPC lines one week post-stroke increases functional recovery within this model. The primary endpoint of this study was to compare the functional recovery of animals treated with test vehicle alone (Plasmalyte-A plus 0.5% human serum albumin) versus animals treated with test vehicle containing NR1 at the same dose (4×10⁵ cells/rat) previously tested with similar hNPCs and found to increase recovery from 1-5 weeks post-treatment. A sensorimotor behavior test known as the Vibrissae-Paw Test was selected as the primary measure of functional recovery, since this test has reproducibly shown increased functional recovery following cortical ischemic stroke and hNPC treatment. Secondary endpoints included an additional behavior test, the Posture-Reflex Test, which has been found to positively correlate with the Vibrissae-Paw Test, assessment of sustainability of functional recovery at three additional time points of behavior testing (6-8 weeks post-transplant), and evaluation of functional recovery and sustainability of functional recovery of a second, lower NR1 dose (100,000 cells/rat) compared with vehicle alone. Tertiary endpoints included comparisons between the two active treatments for a trend in dose effect, a third behavior test (Modified Neurological Score Test) that was investigated as a potential test for functional recovery, as well as several histology-based quantification endpoints, including host brain lesion size and host brain repair pathways, such as neovascularization, inflammation, and increased axonal sprouting by host neurons, referred to as plasticity. These endogenous repair pathways were previously investigated as surrogate measures of recovery and potential hNPC in vivo mechanisms of action. In addition, the survival of transplanted cells in the brain was assessed to characterize NR1 persistence and its potential for post-transplant differentiation, migration, ectopic tissue formation and tumorigenicity.

Male athymic nude rats were subjected to ischemic cortical stroke (ICS) as previously described. At 7 days post-ICS induction, NR1 was administered as an intracerebral (IC) injection at one of two dose levels (1×10⁵ cells/rat or 4×10⁵ cells/rat) in the proposed clinical formulation (Plasmalyte-A plus 0.5% human serum albumin). NR1 administration was performed using a Hamilton microsyringe modeled after the intended clinical delivery device at four injection sites medial to the presumed stroke lesion, with 2.5×10⁴ or 1×10⁵ cells injected in 1 μL at each site. A separate cohort of animals was injected with vehicle control (Plasmalyte-A plus 0.5% human serum) using the same injection method. Treatment groups are shown in Table 1. Behavior testing of sensorimotor function was performed 1 week prior to stroke injury and weekly thereafter for up to 8 weeks post-treatment by an operator who was blind with respect to treatment group. In addition, general health of the animals was monitored weekly by clinical observations and recording of body weights. Following completion of in-life assessments, brain tissue was collected at 9 weeks post-treatment for subsequent histological evaluation.

TABLE 5
Treatment Groups

| | | Sample | NR1 Dose | Treatment | Termination |
|---|---|---|---|---|---|
| Group | Treatment | Size | (cells/rat)^{a} | Time Point^{b} | Time Point^{c} |
| A | NR1 Low Dose | 11 | 1 × 10⁵ | Day 7 | 9 Weeks |
| B | NR1 High Dose | 12 | 4 × 10⁵ | Day 7 | 9 Weeks |
| C | Vehicle | 12 | 0 | Day 7 | 9 Weeks |

^{a}The concentration of NR1 was varied between 0, 2.5 × 10⁴, and 1.0 × 10⁵ cells/μL to allow for a fixed injection volume of 4 μL per rat for each treatment group.
^{b}Time post-stroke
^{c}Time post-treatment

TABLE 6
In-Life and Post-Mortem Assessments and Objectives

| Assessment Tool | Objectives & Assessment Time Point |
|---|---|
| In-Life Assessment Tools: | Weekly from 0-8 weeks after treatment with NR1 or |
| | vehicle: |
| Vibrissae-Paw Test | Measure sensorimotor function after stroke and recovery |
| | after NR1 treatment. |
| Posture-Reflex Test | Measure motor coordination after stroke and recovery after |
| | NR1 treatment. |
| Modified Neurological Score | Measure motor function and coordination after stroke and |
| Test | recovery after NR1 treatment. |
| Clinical observations & Body | Assess general safety of NR1 administration in |
| Weights | immunosuppressed rats after subcortical ischemic stroke. |
| Post-Mortem Assessment | 9 weeks after treatment with NR1 or vehicle: |
| Tools: | |
| Nissl histology | Measure infarct volume after stroke and NR1 treatment. |
| IF for RECA1 | Measure neovascularization in peri-infarct |
| | tissue after stroke and NR1treatment. |
| IF for Iba1 | Measure microglial/immune cell response in peri-infarct tissue after |
| | stroke and NR1 treatment. |
| IF for SMI-312 | Measure axon density in peri-infarct tissue after stroke and |
| | NR1 treatment. |
| IF for hNUC | Determine NR1 survival and migration post-transplant. |

IF = immunofluorescence-based detection.
RECA-1 = rat endothelial cell antigen 1.
Iba-1 = ionized calcium binding adapter molecule 1(activated microglial/immune cell marker).
SM-312 = pan-axonal neurofilament marker.
hNUC = human nuclear antigen.

NR1 Pilot Lot #1 was manufactured at Stanford University using the same procedures utilized for cGMP NR1 production. NR1 Pilot Lot #1 was expanded to p18 from the City Hope p15 Master Cell Bank and cryopreserved in vapor phase liquid nitrogen until the day of transplantation. Analytical Characterization of NR1 Pilot Lot #1

TABLE 8

| | | | | | | | β- | Tra-1- | | | | |
|---|---|---|---|---|---|---|---|---|---|---|---|---|
| NR1 | | Yield | | | Endo- | CD73^{d} | Tubulin | 60^{d} | | | Strength | |
| Lot | DOM | # | Karyo- | Myco- | toxin^{c} | % | III^{d} % | % | Oct4^{e} | Rex1^{e} | % cells | % |
| ID | 2013 | vials | type^{a} | plasma^{b} | EU/mL | positive | positive | positive | ΔCt | ΔCt | recovered | Viability^{f} |
| Pilot | Mar. | 163 | Normal | Negative | 0.82 | 99 ± | 99 ± | 0.38 ± | −12.5 ± | <−20 | 59 | 91 |
| Lot | 24 | | | | | 0.5 | 0.1 | 0.05 | 0.1 | | | |
| 1 | | | | | | | | | | | | |

^{a}Karyotype analysis performed by Cell Line Genetics (Madison, WI).
^{b}Mycoplasma testing performed in Steinberg Lab at Stanford using Lonza MycoAlert Assay.
^{c}Endotoxin testing performed by Nelson Laboratories (Salt Lake City, UT)
^{d}Results determined by flow cytometry and are shown as average ± standard deviation.
^{e}Results determined by quantitative real-time PCR and are shown as average delta Ct relative to GAPDH ± standard deviation.
^{f}Results determined by Trypan blue exclusion.

Dose Preparation of NR1. On each transplant day, NR1 cells were prepared according to the SOP for Cell Preparation included as Appendix 1 of Appendix 1 (Study Protocol). Briefly, cells were thawed and washed three times in test vehicle. After the final centrifugation, cells were resuspended in test vehicle at a concentration of 1×10⁸ cells/mL or 2.5×10′ cells/mL for high dose and low dose NR1 administration respectively. Cells were kept on ice during the transplantation procedure and were administered within 6.5 hours of preparation. NR1 cell concentration and viability were scored by Trypan blue using a standard hemacytometer after the final wash step pre-transplant, and upon completion of the transplant surgeries.

### Vehicle Control

TABLE 8
The test vehicle used is.

| | Test Vehicle | Vendor & | |
|---|---|---|---|
| Name | Composition | Catalogue Number | Lot Number |
| Plasmalyte-A | 99.5% | Baxter # 282544 | C878264 |
| Human Serum | 0.5% | Talecris Biotherapeutics, | 26NJ6H1 |
| Albumin | | Inc. # 13533-684 | |

### Experimental Methods

Animal Subjects. Adult male Cr:NIH-RNU nude rats (Strain Code 02N01) approximately 5-6 weeks of age were obtained from NCI-Frederick (Frederick, Maryland) and were allowed to acclimate to the housing facility until 8-12 weeks of age (approximately 230-350 grams at the time of surgery). Animals were housed 2-3 per cage and provided irradiated food and autoclaved water ad libitum. Animals were kept on a 12/12 hr light/dark cycle, and behavioral observations were conducted during the daytime under standard lighting. All animals arrived in good health and were considered acceptable for enrollment onto the study. All procedures were performed under protocols approved by the Stanford IACUC.

Induction of Ischemic Cortical Stroke. Animals were subjected to the permanent distal middle cerebral artery (MCA) occlusion model of ischemic stroke as described (Kelly et al. 2004). Briefly, under isoflurane anesthesia, the common carotid arteries (CCA) were exposed by means of a ventral midline incision. The distal MCA was exposed through a burr hole between the left ear and eye, permanently occluded by cauterization with bipolar forceps, and cut just above the rhinal fissure. The CCAs were transiently occluded by applying microaneurysm clips to each vessel for 30 min. The clips were then removed, the wound closed, and the animals allowed to recover until fully ambulatory before returning to their home cage.

Assignment of Animals into Treatment Groups. Animals were assigned to treatment groups based on their post-stroke, pre-transplant baseline behavior scores such that the mean and standard deviation of deficit was equivalent as possible across the three treatment groups. The Vibrissae-Paw Test was used as the primary determinant of treatment group assignment, since this test previously exhibited robust deficit and a capacity for recovery post-treatment (Horie et al. 2011, Andres et al. 2011). Animals were distributed across the treatment groups (vehicle, 1×10⁵ NR1, 4×10⁵ NR1) such that the mean scores and standard deviation for the Vibrissae-Paw Test on day 6 post-stroke for each treatment group were as close to equivalent as possible. In cases where two animals have the same deficit score on the Vibrissae-Paw Test, the Posture-Reflex Test was then used to determine their treatment group assignments. In addition, only animals that met the following criteria were enrolled in the study: No observed deficit in the Vibrissae-Paw Test or Posture-Reflex Test prior to stroke induction. Loss of right forelimb function in >7/10 trials in the Vibrissae-Paw Test on day 6 post-Non-severe weight loss during days 1-6 post-stroke (<40% body weight).

Surgery for Cell Transplantation. Seven days after ischemic stroke induction, test vehicle, 1×10⁵ NR1 cells, or 4×10⁵ NR1 cells was stereotaxically infused into 4 sites within the cortex medial to the stroke lesion. With the animals under isoflurane anesthesia, a midline skin incision was made on the skull and the skin was retracted. Burr holes were drilled through the cranium manually using a 25-gauge needle to permit the introduction of a pump-controlled 26-gauge microsyringe needle at the following 4 sites: 1) anterior-posterior (AP): +1.0 mm, medial-lateral (ML): +1.2 mm, dorsal-ventral (DV, from dura): −1.5 mm; 2) AP: −0.3 mm, ML: +1.2 mm, DV: −1.4 mm; 3) AP: −1.8 mm, ML: +1.2 mm, DV: −1.4 mm; 4) AP: −2.8 mm, ML: +1.4 mm, DV: −1.4 mm. test vehicle with or without cells was administered at 0.5 μL/min for a total injection volume of 1 μL delivered over 2 min per site. After the infusion, the microsyringe needle was left in place for an additional 2 min before being carefully retracted. After completion of all four infusions the exposed area is closed with sterile sutures.

Animal Post-Operative Care and Maintenance. Prior to stroke surgery until one week post-transplantation, rats were given Ampicillin (Ampicillin Capsules, USP, DAVA Pharmaceuticals, Inc.) in their drinking water (1 g/L) to reduce risk of infections. Following stroke and transplantation surgeries, rats were inspected daily for dehydration, apparent discomfort, autophagy, or surgical complications. Any health complications were recorded at the time of observation and animals were given appropriate care by Animal Facility technicians under veterinary guidance. Body weights were recorded prior to surgery, daily for one week following stroke surgery, and weekly thereafter.

Methods for Behavioral Testing of Sensorimotor Function. Behavioral testing was used to assess sensorimotor and motor recovery at baseline (pre-stroke and 6 days post-stroke/pre-transplant) and weeks 1-8 post-transplant (2-9 post-stroke). Rats were trained on all behavior tests for three days prior to study initiation and pre-injury baseline testing. Each rat was assessed sequentially in the Vibrissae-Paw Test, Posture-Reflex Test, and Modified Neurological Score Test, with the total battery of tests on each testing day lasting <10 minutes per rat. All behavioral testing was performed by an operator who was blind with respect to treatment group.

Vibrissae-Paw Test. The Vibrissae-Paw Test was used to test the animal's capacity to reflex with their forelimb upon stimulation of the ipsilateral whiskers, and therefore assessed both sensory and motor function (Schallert et al. 2000). Following induction of ischemic stroke in the left somatosensory motor cortex, animals exhibit impaired reflex on the right side, while reflex capacity on the left side remained intact (thus serving as a positive control). To perform the test, animals were held, restraining all limbs except for the testing forelimb, and the ipsilateral whisker was brushed against the corner of a table to assess reflex capacity. On each day of testing, 10 trials per side were performed, thus a score of ‘10’ indicated no deficit, whereas a score of ‘0’ indicated complete loss of function.

Posture-Reflex Test. The Posture-Reflex Test was used to measure the animal's capacity to use both forelimbs symmetrically when being lowered onto a table, as well as its capacity to withstand side-to-side pushing while stationary (Andres et al 2011). To perform the test, animals were held at the base of the tail and lowered to a table surface to observe symmetric versus asymmetric forelimb touchdown. Asymmetric forelimb touchdown was given a deficit score of ‘1’, and symmetric touchdown was scored as ‘0’ (no deficit). Animals were then placed on the table and gently pushed from side-to-side. Inability to resist pushing was given an additional deficit score of ‘1,’ thus the maximum possible deficit score=2.

Modified Neurological Score Test. The Modified Neurological Score Test used was an adaptation of a previously described method (Chen et al. 2001) and was conducted as an exploratory endpoint in this study. This test measured the capacity of both sensory and motor function through a battery of behavioral tests that were scored cumulatively to give a final deficit score. Points were awarded when a subject was unable to perform a test or lacked a given reflex, resulting in a maximum possible deficit score of 14 based on the functional measures.

Histological Processing, Staining, and Immunohistochemistry. At 10 weeks post-stroke, rats were examined carefully for external abnormalities including palpable masses. Under isoflurane anesthesia, rats were transcardially perfused with saline, followed by 4% paraformaldehyde. Brains were dissected, post-fixed in 4% paraformaldehyde, and then cryopreserved in 30% sucrose/PBS for 72 hrs. In addition, the following peripheral organs were collected after perfusion, cryopreserved in the same manner, and stored at −80° C. for possible future examination: lung, heart, spleen, liver. A Leica cryostat was used to prepare free-floating 40 μm coronal brain sections for subsequent histological analyses. All histological analyses were performed blind with respect to treatment group.

Quantification of Infarct Size by Nissl Stain. Six cryoprotected sections per brain that spanned the infarct region (960 μm increments between each section) were mounted on Superfrost Plus microscope slides (VWA) and air-dried at room temperature (RT) for 1 hour. Slide-mounted brain sections were then incubated in the following solutions at RT: 95% ethanol (EtOH) for 2 min, 100% EtOH for 2 min, toluene for 5 min, 95% EtOH for 2 min, 95% EtOH for 2 min, water for 1 min, cresyl violet solution (1% cresyl violet acetate/0.25% glacial acetic acid in water) for 15 min, water for 10 dips at 10 sec/dip, 70% EtOH for 1 min, 95% EtOH for 1 min, 100% EtOH for 1 min, toluene for 5 min, fresh toluene for 5 min. Stained sections were immediately coverslipped with DPX mounting medium (Sigma) and air-dried overnight at RT in a fume hood.

Stained sections were imaged on a flat-bed scanner at 300 dpi and resulting images were analyzed using ImageJ software (NIH) to obtain area measurements of intact (positive for Nissl stain) and lesioned (negative for Nissl stain) tissue. Infarct size for each animal was quantified as the difference of the total hemispheric area of the left, lesioned hemisphere relative to the total hemispheric area of the intact, right hemisphere across the six brain sections as described (Kelly et al. 2004).

Quantification of Blood Vessel Density by Immunofluorescent Staining. For each rat, blood vessel density (BVD) was examined as a measure of relative neovascularization in peri-infarct tissue adjacent to the injury site by immunofluorescent staining with the endothelial-specific antibody, rat endothelial cell antigen-1 (RECA1, Abcam catalogue #AB9774).

To perform immunofluorescent staining, free-floating brain sections were washed in PBS and treated with pre-heated antigen retrieval solution (10 mM sodium citrate/0.05% tween-20/PBS, pH 6.0) at 65° C. for 20 min. Sections were then incubated in blocking solution (2% normal goat serum/1% bovine serum albumin/0.5% triton x-100 in PBS, pH 7.2-7.4) at RT for 1 hr. Blocking solution was removed and sections were incubated with primary antibody (anti-RECA1) in blocking solution (1:200) at 4° C. overnight. Sections were washed 3×10 min in PBS on an orbital shaker and then incubated with secondary antibody (goat-anti-rabbit IgG-Alexa555, Invitrogen catalogue #A-21428) diluted at 1:250 in PBS at RT for 2 hr on an orbital shaker. 4′,6′-diamidino-2-phenylindole (DAPI, 0.5 μg/mL, Sigma) was added during the last 5 min of the secondary antibody incubation period to counterstain nuclei. Sections were washed 3×5 min in PBS, mounted on Superfrost Plus microscope slides (VWA), air-dried at RT for 10-20 min, coverslipped with polyvinyl alcohol (PVA, Sigma), air-dried at RT for 1 hr, and stored at 4° C. in the dark until imaging.

Imaging was performed on a Zeiss fluorescent microscope, and for each brain section, two images within peri-infarct tissue were acquired (6 sections per rat, total of 12 images). For each image, BVD was measured using ImageJ software (NIH) to determine the mean fluorescent area (MFA) of thresholded RECA-1 positivity. The average MFA across all images for each rat was then calculated, and treatment group means were compared statistically.

Quantification of Microglial/Immune Cell Activation by Immunofluorescent Staining. For each rat, microglial/immune cell activation was quantified in peri-infarct tissue adjacent to the injury site by immunofluorescent staining with an antibody against ionized calcium binding adapter molecule 1 (Iba1, Waco Chemicals USA catalogue #019-19741).

Immunofluorescent staining was performed on free-floating brain sections and corresponding peri-infarct images were acquired as described for quantification of BVD above. For each image, microglial/immune cell activation was measured using ImageJ software (NIH) to determine the mean fluorescent area (MFA) of thresholded Iba1 positivity. The average MFA across all images for each rat was then calculated, and treatment group means were compared statistically.

Quantification of Axon density by Immunofluorescent Staining. For each rat, axon density was quantified at the lesion border by immunofluorescent staining with a pan-neurofilament antibody (SM1312, Abcam catalogue #ab24574).

Immunofluorescent staining was performed on free-floating brain sections as described for quantification of BVD above, and images of the lesion border were acquired (2 images/section, 12 images/rat). For each image, axon density was measured using ImageJ software (NIH) to determine the mean fluorescent area (MFA) of thresholded SM312 positivity. The average MFA across all images for each rat was then calculated, and treatment group means were compared.

Quantification of NR1 Survival by Immunofluorescent Staining. Survival and migration of transplanted NR1 cells in the stroke injured-brain was investigated by immunofluorescent staining using an antibody specific for human nuclei (anti-hNUC, Millipore catalogue #MAB1281). Sections corresponding to the 4 transplant sites and sections that contained a visible needle track, were used for staining. In the event that any potential hNUC positivity was detected, additional sections at 80 μm intervals in both directions AP were stained (i.e., 1 adjacent section at each interval was saved for co-staining studies) until no hNUC positivity was detected.

Immunofluorescent staining was performed on free-floating brain sections as described for quantification of BVD. The primary antibody (anti-hNUC) was diluted at 1:200 and detected using a goat-anti-mouse IgG-Alexa488 secondary antibody (Invitrogen catalogue #A11001) diluted at 1:250. 4′,6′-diamidino-2-phenylindole (DAPI, 0.5 μg/mL, Sigma) was added during the last 5 min of the secondary antibody incubation period to counterstain nuclei. For each set of stained slides, vehicle-treated negative control brain sections were included, as were positive control brain sections from a separate cohort of animals previously confirmed to contain transplanted human embryonic kidney cells (HEK 293T cells).

All stained sections were manually screened for hNUC positivity using a Zeiss epifluorescent microscope and without knowledge of treatment groups. For each animal, a minimum of four brain sections was stained with hNUC, and for each stained section, the entire tissue was scanned for hNUC positivity.

Data Analysis and Statistical Methods. Mean body weights for each treatment group were compared by Repeated Measures ANOVA using JMP software (V 11.0.0, SAS Institute, Inc., Cary, NC).

All behavior testing was performed by an operator who was blind with respect to treatment group. For analysis of each behavior test, individual animal's behavior scores at each time point post-treatment were normalized to their post-stroke/pre-treatment baseline (1 week post-stroke). Normalized behavior scores were then used to calculate treatment group means at each time point.

For the Vibrissae-Paw Test, statistical comparisons were performed for the 4×10⁵ NR1 and vehicle treatment group means at 1-5 weeks post-treatment (2-6 weeks post-stroke) by Multiple Comparisons ANOVA with Bonferonni post-host testing using JMP software (V 11.0.0, SAS Institute, Inc., Cary, NC). In addition, statistical analysis of the full behavior time course (1-8 weeks post-treatment) was performed for all three treatment groups by Multiple Comparison ANOVA with Bonferroni post hoc testing using JMP software (V 11.0.0, SAS Institute, Inc., Cary, NC). Non-parametric statistical analysis was performed on the sum Vibrissae-Paw score for 4×10⁵ NR1 and vehicle treatment group means at weeks 1-5 post-treatment using the Mann-Whitney test in R (R Development Core Team, 2010). In addition, non-parametric analysis of the full behavior time course (1-8 weeks post-treatment) was performed for the 4×10⁵ NR1 and vehicle treatment groups using the Wilcox Multiple Comparison Test with Benjami-Hochberg Adjustment in R (R Development Core Team, 2010).

For the Posture-Reflex Test and Modified Neurological Score Test, statistical analysis of the full behavior time course (1-8 weeks post-treatment) was performed for all three treatment groups by Multiple Comparison ANOVA with Bonferroni post hoc testing using JMP software (V 11.0.0, SAS Institute, Inc., Cary, NC).

All histology data collection and processing was performed by an operator that was blind with respect to treatment group. Following image acquisition and data processing, treatment groups were unblinded for statistical comparisons. For each histology endpoint, high dose and lose dose NR1 treatment group means were individually compared to the vehicle group means by Student's t-test in JMP software (V 11.0.0, SAS Institute, Inc., Cary, NC).

Linear regression analysis of each individual animal's behavior score in the Vibrissae-Paw Test at 8 weeks post-treatment and histological endpoints was performed using JMP software (V 11.0.0, SAS Institute, Inc., Cary, NC).

### Results Summary

Animal Fate and Disposition. A total of 42 animals underwent ischemic stroke surgery, of which 7 died prior to transplantation surgery or did not meet study inclusion criteria. Of the animals that met study inclusion criteria, 12 animals received 4×10⁵ NR1 cells, 11 animals received 1×10⁵ NR1 cells, and 12 animals received vehicle. All animals that underwent transplantation surgery survived and remained on study until the scheduled termination at 9 weeks post-transplantation.

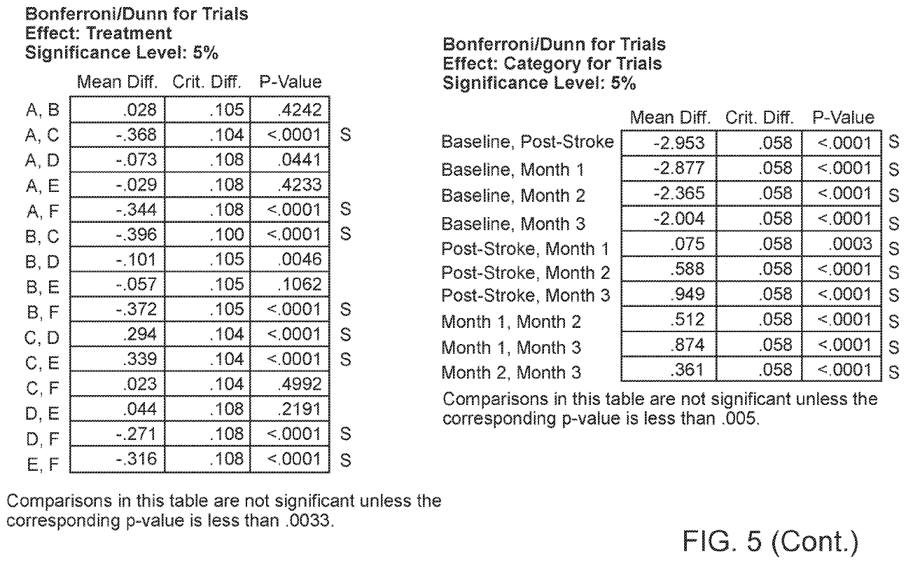

FIG. 7: Vibrissae-Paw Test Raw Data with Heat Map Overla.Heat map depicting the raw behavior data from the Vibrissae-Paw (V-P) Test for each individual animal within each treatment group (individual animals IDs are listed in column 1). A score of ‘10’ (yellow) indicates full function on this test (i.e., no deficit) as seen for all animals pre-stroke (column 2). A score of ‘0’ (dark grey) indicates complete loss of function (i.e., full deficit), as seen for most animals following stroke induction (red vertical arrow) on the day prior to transplant.

For statistical analysis, each animal's post-transplant scores were normalized to its pre-transplant baseline score, and group means were compared. Statistical analysis of the primary endpoint (1-5 weeks post-treatment, 2-6 weeks post-stroke) by Multiple Comparison ANOVA with Bonferonni post-hoc testing indicated a significant effect of 4×10⁵ NR1 cells relative to vehicle treatment beginning at 4 weeks post-treatment. These results were further corroborated by non-parametric analysis using the Mann-Whitney test of the sum Vibrissae-Paw score for weeks 2-6 post-stroke, which indicated a significant effect of treatment with 4×10⁵ NR1 cells relative to vehicle (p=0.0106). All animals were assessed in the Vibrissae-Paw Test at three additional time points beyond the primary endpoint to test for sustained functional recovery (7-9 weeks post-stroke). Statistical analysis of the full behavior time course (2-9 weeks post-stroke) by Multiple Comparison ANOVA with Bonferroni post hoc testing indicated a significant effect of treatment with 4×10⁵ NR1 cells relative to vehicle at 7, 8 and 9 weeks post-stroke (p=0.0004, 0.0021 and 0.0001, respectively). Non-parametric analysis using the Wilcox Multiple Comparison Test with Benjami-Hochberg Adjustment, indicated a significant effect of treatment with 4×10⁵ NR1 cells at 5, 6, 7 and 9 weeks post-stroke (p=0.0469). In contrast, no significant treatment related effects were observed for animals treated with 1×10⁵ NR1 cells relative to the vehicle control group.

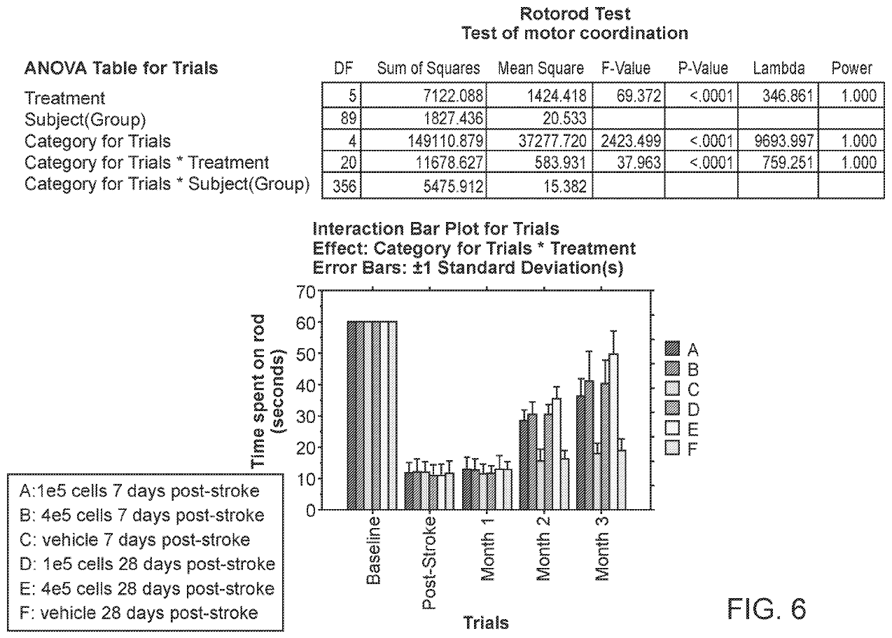

FIG. 8: For each treatment group, mean functional recovery in the V-P Test is shown relative to time. For each animal, functional recovery was calculated as their V-P score at a given time point post-treatment normalized to their pre-treatment baseline (Week X-Week 1 V-P score). Error bars=standard error of the mean (SEM). *, **, ***indicates p<0.05, p<0.01, p<0.001, respectively, for 4×10⁵ NR1 treatment relative to vehicle control by Multiple Comparison ANOVA with Bonferroni post hoc testing.

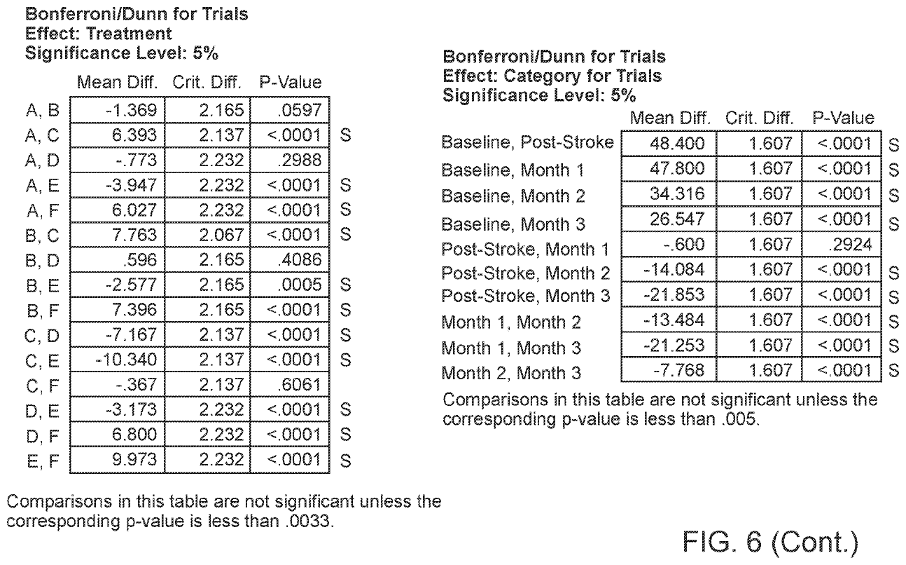

Posture-Reflex Test. The Posture-Reflex Test was used as an additional secondary measure of motor function and motor coordination post-stroke and NR1 treatment. Similar to the Vibrissae-Paw Test, significant effects were observed in the Posture Reflex Test with treatment of 4×10⁵ NR1 relative to the vehicle control group at 5-9 weeks post-stroke by Multiple Comparison ANOVA with Bonferroni post hoc testing (p=0.0194, 0.0115, 0.0007, 0.0028 and 0.0003 for 5, 6, 7, 8 and 9 weeks post-stroke, respectively). Results are shown in FIG. 9.

FIG. 9: Mean functional recovery in the Posture-Reflex Test relative to time for each treatment group. To calculate mean functional recovery at each time point post-treatment, raw Posture-Reflex scores for each animal were normalized to their pre-treatment baseline (Week X-Week 1), and the average recovery for each treatment group was determined. Error bars=SEM. *, **, ***indicates p<0.05, p<0.01, p<0.001, respectively, for 4×10⁵ NR1 treatment relative to vehicle control by Multiple Comparison ANOVA with Bonferroni post hoc testing.

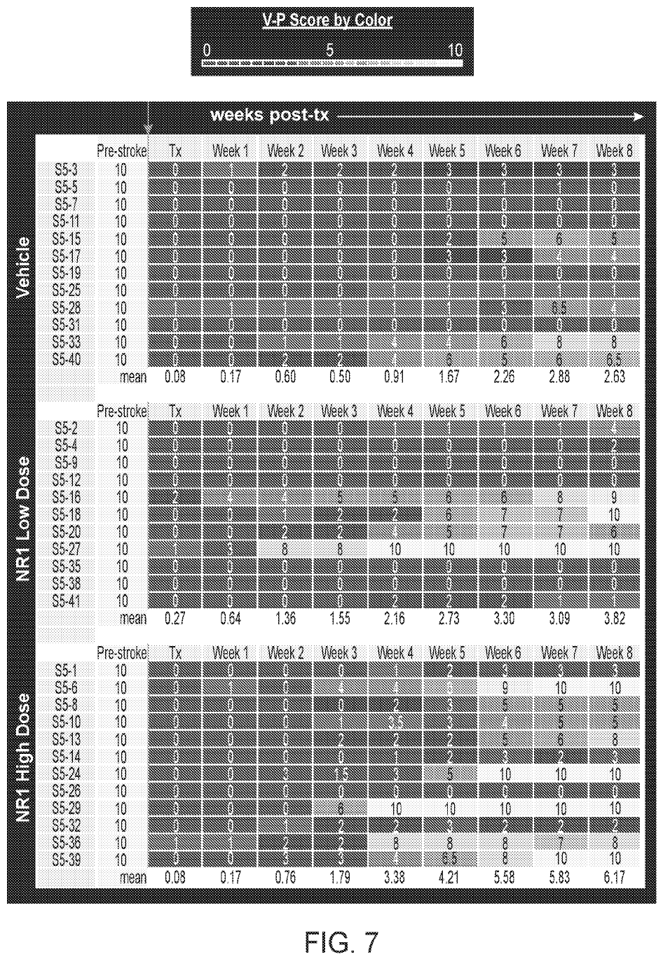

Modified Neurological Score Test. The Modified Neurological Score Test was used to measure the capacity of both sensory and motor function through a battery of behavioral tests (see Table 5, in Section 3.6.3) and served as an additional exploratory endpoint in this study. Results were analyzed in the same manner as the other behavior tests, normalizing each post-treatment score to the individual's pre-treatment baseline and comparing group means by Multiple Comparisons ANOVA. In contrast to the Vibrissae-Paw Test and Posture-Reflex Test, no significant differences were observed between treatment groups in the Modified Neurological Score Test. Results are shown in FIG. 10.

FIG. 10: Mean functional recovery in the Modified Neurological Score Test relative to time for each treatment group. To calculate mean functional recovery at each time point post-treatment, raw cumulative scores for each animal were normalized to their pre-treatment baseline (Week X-Week 1), and the mean recovery for each treatment group was determined for each time point. Error bars=SEM.

Anatomical Outcome Measures. Upon completion of behavior testing, animals were perfused 9 weeks post-transplant (10 weeks post-stroke), and brain tissue was examined histologically. Parallel sets of coronal brain sections (40 μm) were used to quantify infarct size and to obtain quantitative measures of blood vessel density, microglial/immune cell activation, and axon density in peri-infarct cortex. In addition, sections containing visible needle tracks or approximate transplant coordinates were assessed for NR1 persistence, as well as any treatment-associated tumor or ectopic tissue formation.

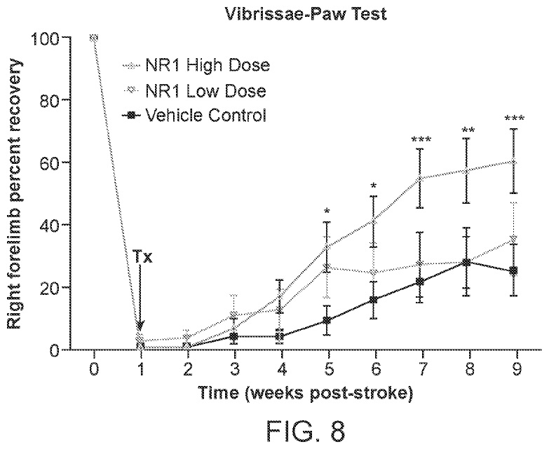

Representative Images of Histological Endpoints. Representative photomicrographs of cresyl violet stained sections are shown in FIG. 11, providing examples of moderate and severe infarcts in animals treated with vehicle or 4×10⁵ NR1 cells.

FIG. 11: Representative photomicrographs of cresyl violet-stained coronal brain sections showing infarct size at 10 weeks post-stroke. Two animals each from the vehicle group and the 4×10⁵ NR1 group are shown, reflecting moderate (Animal Numbers 36, 40) or severe (Animal Numbers 10, 31) infarcts in the left cerebral cortex. In some cases, additional tissue loss appears to have resulted from enlargement of the lateral ventricle (e.g., Animal Numbers 10, 36, 40).

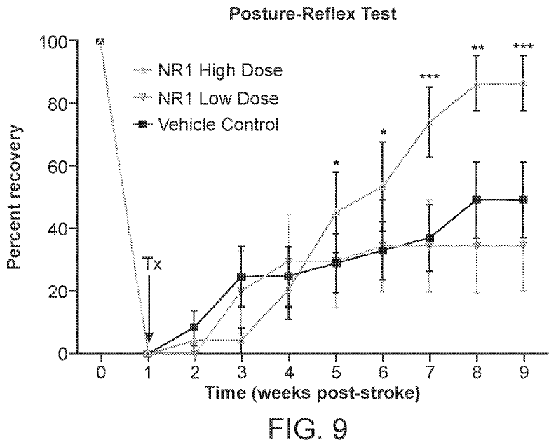

To investigate the potential effects of NR1 treatment on host brain repair, peri-infarct cortex was assessed for blood vessel density, microglial/immune cell activation, and axon density by immunofluorescent labeling with RECA1, Iba1, and SMI-312, respectively. Representative images of each stain for animals treated with vehicle or 4×10⁵ NR1 cells are shown in FIG. 12.

FIG. 12. Representative photomicrographs of immunofluorescent staining in peri-infarct cortex at 9 weeks post-treatment (10 weeks post-stroke). Four representative animals are shown, including two vehicle-treated animals (Animal Numbers 15 and 40, rows 1 and 2), and two animals treated with 4×10⁵ NR1 cells (Animal Numbers 36 and 39, rows 3 and 4). For each animal, figure panels show immunofluorescent labeling of 1.) blood vessels with anti-RECA1 antibody (left column), 2.) activated microglial/immune cells with an anti-Iba1 antibody (middle column), and 3.) axon density with an SMI-312 antibody (right column).

Linear Regression Analyses of Functional Recovery and Histological Endpoints. To identify potential correlations between functional recovery and anatomical outcome measures, linear regression analyses of behavioral and histological data were performed. Individual animal data from the Vibrissae-Paw Test at 8 weeks post-treatment was plotted separately against each of the following anatomical outcome measures: infarct size, peri-infarct blood vessel density, peri-infarct microglial/immune cell activation, and peri-infarct axon density. Based on this approach, infarct size at 9 weeks post-treatment showed a significant correlation with performance in the Vibrissae-Paw Test at 8 weeks post-treatment for animals in the vehicle and 1×10⁵ NR1 treatment groups, where increased infarct size correlated with decreased performance on the Vibrissae-Paw Test. In contrast, infarct size was not correlated with decreased performance on the Vibrissae-Paw Test for animals in the 4×10⁵ NR1 treatment group, indicative of the significant recovery enhancement observed in this treatment group. No significant correlations were observed between functional recovery at 8 weeks post-treatment and any of the other histological endpoints. For each comparison, corresponding R² values and p values are shown as a function of treatment group in Table 9, Linear Regression Analysis of Vibrissae-Paw Test at 8 Weeks Post-Treatment and Histological Endpoints.

TABLE 9

| Histological Endpoint | Treatment Group | R² Value | P Value |
|---|---|---|---|
| Infarct size | Vehicle | 0.570491 | 0.0072 |
| Infarct size | 1 × 10⁵ NR1 | 0.678955 | 0.0063 |
| Infarct size | 4 × 10⁵ NR1 | 0.222163 | 0.1219 |
| Peri-infarct blood vessel density | Vehicle | 0.042685 | 0.5422 |
| Peri-infarct blood vessel density | 1 × 10⁵ NR1 | 0.064688 | 0.4783 |
| Peri-infarct blood vessel density | 4 × 10⁵ NR1 | 0.00056 | 0.9418 |
| Per-infarct microglial/ | Vehicle | 0.282115 | 0.0927 |
| immune cell activation | | | |
| Per-infarct microglial/ | 1 × 10⁵ NR1 | 0.017363 | 0.7167 |
| immune cell activation | | | |
| Per-infarct microglial/ | 4 × 10⁵ NR1 | 0.02389 | 0.6315 |
| immune cell activation | | | |
| Peri-infarct axon density | Vehicle | 0.00182 | 0.9009 |
| Peri-infarct axon density | 1 × 10⁵ NR1 | 0.001686 | 0.9103 |
| Peri-infarct axon density | 4 × 10⁵ NR1 | 0.273282 | 0.0990 |

Quantification of Mean Infarct Size by Treatment Group. For each animal, a set of six coronal sections was stained with cresyl violet and the total intact tissue in each hemisphere of each section was measured using ImageJ software (NIH) by an operator that was blind with respect to treatment group. The sum of intact tissue across all six sections was calculated for each hemisphere, and infarct size was calculated as [(1-(total area of intact tissue within the lesioned hemisphere/total area of uninjured contralateral hemisphere))*100]. No significant differences in infarct size were observed for the 1×10⁵ NR1 treatment group or 4×10⁵ treatment group relative to the vehicle group by Student's t-test (1×10⁵ NR1 versus vehicle, p=0.3825; 4×10⁵ NR1 versus vehicle, p=0.888).

Quantification of Anatomical Outcome Measures in Per-Infarct Cortex. For quantification of anatomical outcome measures in peri-infarct cortex, four coronal sections spanning the lesion were processed for immunofluorescent staining, and for each section, two imaging fields within the ventral peri-infarct cortex were acquired (8 imaging fields/animal). Thresholded area measurements of each stain were calculated using ImageJ software (NIH) by an operator that was blind with respect to treatment group. Treatment group means were then compared using JMP 11 (SAS), with the NR1 high dose group (4×10⁵ cells/rat) and the NR1 low dose group (1×10⁵ cells/rat cells/rat) compared separately to the vehicle group by Student's t-Test.

Quantification of Mean Peri-Infarct Blood Vessel Density by Treatment Group. The mean fluorescent area of RECA1 positivity was calculated for each treatment group, and the two NR1 treatment groups were independently compared to the vehicle group by Student's t-test. No significant differences were observed between treatment groups (4×10⁵ NR1 vs. vehicle, p=0.3352; 1×10⁵ NR1 vs. vehicle, p=0.4420).

Quantification of Mean Peri-Infarct Microglial/Immune Cell Activation by Treatment Group. The mean fluorescent area of Iba1 positivity was calculated for each treatment group, and the two NR1 treatment groups were independently compared to the vehicle group by Student's t-test. Based on this analysis, the 4×10⁵ NR1 treatment exhibited a significant reduction in peri-infarct microglial/immune cell activation (p=0.0340). In contrast, there was no significant difference between group means for 1×10⁵ NR1 versus vehicle (p=0.0690).

Quantification of Mean Peri-Infarct Axon Density by Treatment Group. The mean fluorescent area of SMI-312 positivity was calculated for each treatment group, and the two NR1 treatment groups were independently compared to the vehicle group by Student's t-test. No significant differences were observed between treatment groups (4×10⁵ NR1 vs. vehicle, p=0.7838; 1×10⁵ NR1 vs. vehicle, p=0.2538).

Assessment of NR1 Persistence. At 9 weeks post-treatment (10 weeks post-stroke), sections containing visible needle tracks or approximate transplant coordinates were assessed for NR1 persistence, as well as any treatment-associated tumor or ectopic tissue formation. NR1 persistence was assessed by immunofluorescent labeling with anti-hNUC. Based on this approach, no hNUC-positive cells were detected in any of the assessed animals at 9 weeks post-treatment. In addition, no signs of ectopic tissue or tumor formation were observed in any of the assessed animals.

### Interpretation of Results

Behavioral Assessment Outcome Measures. Animals were behaviorally assessed pre-injury, post-injury/pre-treatment, and weekly from 1 to 8 weeks post-treatment using the following behavioral tests: Vibrissae-Paw Test, Posture-Reflex Test, Modified Neurological Score Test.

Animals exhibited no deficits in any of the behavior tests prior to stroke injury. Behavioral assessments at 1 week post-stroke injury (pre-treatment) indicated similarly extensive functional deficit based on all three tests, manifest as substantial impairment of sensorimotor function of the contralateral whisker/forelimb reflex system (contralateral relative to stroke side) based on the Vibrissae-Paw Test and lesser impairment of overall motor coordination and balance based on the Posture-Reflex Test and Modified Neurological Score Test.

Vibrissae-Paw Test. To assess the combined sensorimotor function of the contralateral whiskers and forelimb, the Vibrissae-Paw Test was utilized. Results of the Vibrissae-Paw Test are presented in Section 4.3.1. Following stroke injury, most animals exhibited full deficit in this test, failing to respond to contralateral whisker stimulation in 10 out of 10 trials at 1 week post-stroke. Post-treatment, animals that received vehicle exhibited minimal functional recovery on this test, reaching a mean recovery of 28% at 7 weeks and 26% at 8 weeks (8 and 9 weeks post-stroke). Similar recovery was observed for animals treated with 1×10⁵ NR1 cells, with the group mean recovery reaching 31% and 38% at 7 and 8 weeks post-treatment. In contrast, animals treated with 4×10⁵ NR1 cells exhibited a significant increase in functional recovery in the Vibrissae-Paw Test relative to vehicle-treated animals, with the group mean recovery reaching 34% by 4 weeks post-treatment (vehicle group mean=10%) and increasing to 61% at 8 weeks post-treatment (p=0.0469 at 4, 5, 6, and 8 weeks post-treatment by Wilcox Multiple Comparison Test with Benjami-Hochberg Adjustment). The low degree of spontaneous recovery by vehicle-treated animals in the Vibrissae-Paw Test likely reflected the specific location of the stroke lesion for this model, which typically encompasses both the left whisker barrel cortex (responsible for receiving sensory stimuli from the right whiskers) and the motor cortex responsible for controlling the right forelimb. As such, results of this study indicate that the Vibrissae-Paw Test can provide a sensitive measure of functional deficit and recovery for the permanent model of ischemic cortical stroke, and further, that administration of 4×10⁵ NR1 cells, but not 1×10⁵ NR1 cells, medial and immediately adjacent to the lesion site may selectively enhance functional recovery of these brain regions.

Posture-Reflex Test. To provide a second measure of forelimb motor function and a measure of motor coordination, the Posture-Reflex Test was utilized. Results of the Posture-Reflex Test are presented in Section 4.3.2. Following stroke injury, most animals exhibited substantial deficit in this test, with most animals failing to perform symmetrical forelimb touchdown while being lowered onto a table, and many animals exhibiting an impaired ability to resist gentle side-to-side pushing while stationary. Vehicle-treated animals exhibited moderate functional recovery on this test, reaching a mean recovery of 50% at 8 weeks. Similar recovery was observed for animals treated with 1×10⁵ NR1 cells, with the group mean recovery reaching 35% at 8 weeks post-treatment. In contrast, animals treated with 4×10⁵ NR1 cells exhibited a significant increase in functional recovery in the Posture-Reflex Test relative to vehicle-treated animals, with the group mean recovery reaching 46% by 4 weeks post-treatment (vehicle group mean=29%) and increasing to 88% at 8 weeks post-treatment (p=0.0194, 0.0115, 0.0007, 0.0028 and 0.0003 for 4, 5, 6, 7 and 8 weeks post-treatment, respectively, by Multiple Comparison ANOVA with Bonferroni post hoc testing). A greater degree of spontaneous recovery in the vehicle group was observed in this test relative to the Vibrissae-Paw Test, which likely was due to recovery of general motor coordination despite persistence of right forelimb motor deficit.

Modified Neurological Score Test. The Modified Neurological Score Test was utilized as a third exploratory measure of post-stroke functional deficit and recovery, providing a cumulative assessment of forelimb sensorimotor function, limb coordination while walking, and balance (see Table 6 for parameters tested). Results of the Modified Neurological Test are presented in Section 4.3.3. Following stroke injury, most animals exhibited moderate deficit in this test, manifest as impairments in right forelimb flexion, right forelimb sensorimotor function, and ability to navigate a balance beam. In contrast to the other behavioral tests, all treatment groups exhibited substantial recovery in the Modified Neurological Score Test, largely due to improvements in balance beam performance. No significant differences in recovery were observed for animals treated with 1×10⁵ NR1 cells or 4×10⁵ NR1 cells relative to vehicle, likely due to the extent of spontaneous recovery and/or adaptation also observed in vehicle-treated animals.

Anatomical Outcome Measures. For the histological analyses, brain tissues were collected at 9 weeks post-treatment (10 weeks post-stroke) and processed for histological assessment of infarct size, integrity/repair of peri-infarct cortex (determined by blood vessel density, presence of activated microglial/immune cells, and axon outgrowth), human cell persistence, as well as for identification of any ectopic tissue or tumor formation.

In support of the observed impairments of right whisker/forelimb sensorimotor function, stroke-induced infarcts were predominantly restricted to the left somatosensory motor cortex, which included the whisker barrel cortex and the region of motor cortex responsible for control of the right forelimb. Infarct sizes ranged from 7-49% of the total assessed left hemisphere, with some infarcts involving expansion of the ipsilateral lateral ventricle (for examples see FIG. 6). No significant differences in infarct size were observed for either NR1 treatment group (1×10⁵ NR1 cells or 4×10⁵ NR1 cells) relative to the vehicle group.

Histological assessments of peri-infarct cortex at 10 weeks post-stroke were performed to identify changes in host-brain tissue sparing and/or repair that might be correlated with NR1-associated enhancement of post-stroke functional recovery. Immunofluorescent labeling of vasculature (RECA1), activated microglial/immune cells (Iba1), and axons was used to obtain quantitative measures of peri-infarct blood vessel density, inflammation, and axon sparing/outgrowth, respectively. Results are presented in Sections 4.4.2-4.4.8. Based on these analyses, the 4×10⁵ NR1 treatment group exhibited a significant reduction in activated microglial/immune cells relative to the vehicle group at 10 weeks post-stroke (p=0.0340 by Student's t-test). However, linear regression analysis indicated no correlation between each animal's functional recovery at 9 weeks post-stroke (as determined by Vibrissae-Paw Test) and its peri-infarct microglial/immune cell activation, indicating that modulation of inflammation in peri-infarct tissue may not be the predominant mechanism driving the observed recovery enhancement associated with high dose NR1 treatment in this stroke model. Further, histological assessment of peri-infarct blood vessel density and axon sparing/outgrowth indicated no significant differences between treatment groups means and no correlation with individual behavioral recovery at 9 weeks post-stroke by linear regression analyses.

NR1 cell persistence was assessed histology at 9 weeks post-treatment (10 weeks post-stroke) by immunofluorescent labeling with anti-human nuclei antibody (hNUC). Results are presented in Table 9. For each animal treated with NR1, brain sections containing visible needle tracks as well as those containing the target transplant coordinates were manually assessed for the presence of hNUC positive cells. For all assessed animals, no hNUC positive cells were detected at 9 weeks post-treatment, suggesting that NR1 cells were unable to persist long-term in this xenograft injury model. In addition, no signs of ectopic tissue or tumor formation were observed in any of the assessed animals.

### Conclusions

The objective of the current study was to assess the functional and anatomical effects of NR1 transplantation 7 days after ischemic cortical stroke in immunocompromised rats. Behavioral assessments and histological assessments of brain tissue confirmed that the ischemic injury was unilateral and that the most pronounced functional losses were observed in the function of the contralateral (right) forelimb. Administration of either NR1 dose did not adversely affect animal health, body weight or mortality for the duration of the study.

Treatment with 4×10⁵ NR1 cells, but not 1×10⁵ NR1 cells, resulted in improved sensorimotor function of the contralateral whiskers and forelimb compared to vehicle controls, indicating a potential dose effect of NR1 in this model.

Histological assessment at 10 weeks post-stroke indicated that animals treated with 4×10⁵ NR1 cells, but not 1×10⁵ NR1 cells, had reduced microglial/immune cell activation in peri-infarct cortex relative to vehicle controls based on treatment group means, however this result did not correlate with individual functional recovery at 8 weeks post-treatment.

For all assessed animals, no surviving human cells were detected at 9 weeks post-administration, suggesting that NR1 cells were unable to persist long-term in this xenograft injury model. Improved behavior recovery was observed in animals treated with 4×10⁵ NR1 cells beginning at 4 weeks post-treatment and persisting until at least 8 weeks post-treatment (study end), indicating that chronic recovery enhancement occurred in the absence of long-term NR1 persistence. No ectopic tissue or tumor formation was observed in any of the assessed animals.

### Example 5

NR1 is the drug product. It is made from NR1 Master Cell Bank (MCB), going through NR1 Drug Substance (DS) to make the product, NR1. Table 2 provides the names of the various cell bank and drug materials used.

NR1 is an expandable human neural stem cell line, derived from the WiCell Research Institute WA09 (H9) human Embryonic Stem Cell (hESC) line. NR1 forms expandable and homogenous long-term cultures in vitro without loss of phenotypic potential or karyotype stability. The manufacturing process for NR1 does not require repeated use of hESCs and differentiation into neural stem cells, rather vials of NR1 Master Cell Bank (MCB) cryopreserved at passage 15 (p15) are thawed and expanded in defined culture media in feeder-free adherent culture to p18 to produce NR1 Drug Substance (DS).

One vial of drug product contains 6×10⁶ NR1 neural stem cells as the active substance. The cells are cryopreserved in 50% of the medium that they were expanded in, and 50% cryopreservation medium composed of a commercially available cryopreservation solution (85%) (ProFreeze CDM, Lonza) and DMSO (15%).

Dependent on dose escalation cohort, 2 to 12 vials will be thawed per patient, cells will be washed and concentrated into an administration formulation of PlasmaLyte-A™ with 0.5% HSA at various cell concentrations with injection volume being held constant throughout the dose escalation. Cells will be administered using stereotactic intracranial injection.

The indication is for use in a combined Phase 1/Phase 2 clinical study that has been designed to assess the safety, tolerability, and obtain an indication of efficacy of intra-cerebral transplantation (ICT) of NR1 in subjects with chronic ischemic subcortical stroke (cISS). It is proposed as an open-label safety and tolerability study using stereotactic, intracranial injection of NR1 cells in subjects 18 to 75 years with hemiparesis from stable ischemic stroke.

### Pharmaceutical Development

Process development was not segregated between drug substance and product, as the manufacturing of drug product NR1 is one continuous process with minimal in-process controls ending with vialing and cryopreservation.

An exploratory research study was performed to investigate cGMP MCB derived NR1 drug product growth behavior at the End Of Production (EOP). Only one culture of p18 NR1 was grown out to these higher passage numbers. This investigation addressed two areas of production; change of operator effect and number of passages to reach zero growth. 1) EOP run was performed at Stanford by a technician that was different from the technician that grew the seven pilot lots and one research lot, and 2) EOP investigated how many passages can occur before specific growth rate reaches zero.

### Karyotype Analysis of EOP Samples

To determine if karyotype stability was maintained as cells were grown past p18, cryopreserved cell samples from p28 were sent out to Cell Line Genetics, Madison, WI on 26 June, 2014 for karyotype analysis. Results indicated that cells up to p28 were of normal karyotype. A representative sample of p30 material was not submitted for karyotype evaluation, as the theoretical yield was so large by this passage it was determined that this number of passages would likely never occur in cGMP or non-clinical production. It is unlikely that between passages 18 and 28, cells converted to abnormal then reverted to normal karyotype.

### Microarray and Immunohistochemical Analysis of EOP Samples

Additionally, as part of this exploratory study, samples of cells at passages 18, 24, 28 and 30 were sent to Dr. William Lowry at UCLA to evaluate the transcriptomes and in vitro differentiation characteristics of NR1 EOP cells. The techniques were not the qualified methods that are currently being used for conducting release testing of cGMP NR1. Rather, these studies were either 1) tissue immunohistochemical staining of cultured cells from one culture per passage time point that were scored based on qualitative observations of the density or brightness of immunofluorescence staining, or 2) research-based microarray analyses whose reproducibility has not been determined.

Briefly, the results from this preliminary experiment suggest that NR1 cells from the different passages cluster transcriptionally to each other and are more similar to (but uniquely different from) fibroblasts than any other cell type (hESC, hiPSC, NSC, blood vessel endothelial, smooth muscle, keratinocyte, mesothelial, hepatocyte, neurons, kidney and myoepithelial) profiled. In addition, among the NR1 passage samples provided, the earlier passages appeared to be distinct from the later passages, indicating some selection over time. NR1 at p18 show individual gene expression profiles comparable to those shown by p24-30, and p24-30 cluster together as do p18 NR1 DS cells. Despite being derived from an hESC line, there was no evidence either at the RNA or protein level that key pluripotency genes were reactivated in any of the NR1 passages analyzed. NR1 cells at all passages tested showed no difference in their limited ability to differentiate into neurons, astrocytes or oligodendrocytes.

### Manufacturing Process

NR1 is formed from expandable and homogenous cultures that are stable and can be passaged at least 15 times without loss of phenotypic potential or karyotype stability. The manufacturing process for NR1 does not require repeated use of hESCs and differentiation into neural progenitor cells, rather, vials of NR1 Master Cell Bank (MCB) cryopreserved at passage 15 (p15) are thawed and expanded in defined culture media in feeder-free adherent culture to p18 to produce NR1 Drug Substance (DS). No vials of NR1 DS are produced, as such, because the manufacture of the DS is part of a continuous process that is followed by the fill, finish and cryopreservation of the DS into the NR1.

cGMP production of drug substance and drug product at COH is initiated with generation of the cGMP NR1 MCB, which was vialed and cryopreserved on Sep. 1, 2012. From this “source” MCB material, thus far, three cGMP runs have been performed to manufacture the NR1. The drug product results from a continuous process from drug substance. A schematic diagram (FIG. 2) of the entire process is provided here to assist in delineation of drug substance and drug product steps within the continuous production. For clarity, drug substance and drug product delineation is indicated by a black horizontal line within the diagram, as well as noted in the vertical first panels. Cells at p15, p16 & p17 are plated upon NR1 MCB thaw and subsequent expansions, but the passage number increases by one upon enzymatic release and harvest. During cell expansion, culture passage numbers increase by one, after the monolayer is exposed to trypsin; (e.g., when passage 16 cells are seeded, then expanded into a near confluent monolayer and trypsinized, the resulting suspension is considered passage 17).

Bulk Drug Substance (BDS) is considered to be the final passage (p18) harvest by enzymatic cell culture vessel release and initial pooled cell resuspension, which is counted to determine cell concentration and total cells harvested. At that time, final formulation of p18 begins for NR1 vialed and cryopreserved preparation.

The drug product, NR1, production process initiates with the initial harvested, centrifuged, resuspended and pooled cells that are the trypsin digested (released) from passage 17 cells seeded 4 days prior.

All of the process steps from BDS to fill finished frozen NR1 occur on the same day as the BDS harvest. Briefly, cells are counted to calculate harvested cell concentration and total cell yield. Cells are then centrifuged, the supernatant removed and cells resuspended in complete medium at 1.2×10⁷ cells/mL. Cells are re-counted to assure cell concentration fill accuracy, then diluted 1:1 (v:v) with 2×ProFreeze/DMSO freezing solution to result in a final DMSO concentration of 7.5%. This cell suspension is then manually filled at nominal volume of 1 mL per polypropylene cryovial. Vialed NR1 is cryopreserved by freezing using a controlled rate cryofreezer. NR1 (the drug product) is stored in vapor phase LN₂ cyrofreezers.

### Control of Critical Steps and Intermediates

Critical steps in drug product manufacturing include cell enumeration, centrifugation and controlled rate cyropreservation.

Cell enumeration: Cells are counted manually using trypan blue dye for visualization and assessment of cell viability.

Centrifugation: Centrifugation is controlled by batch record specification for speeds specified as NNN×g, which can be calculated for any centrifuge/rotor combination. Equipment is installed under cGMP IOQ documentation and is calibrated and maintained under COH PM and calibration programs.

Controlled Rate Cryopreservation: Drug Product is cryopreserved in 1.2 mL cryovials (Section 3.2.P.7) utilizing a Planar Controlled Rate Freeze. COH uses their “standard” 1.0 mL cryopreservation freezing program which reduces the chamber temperature approximately 1° C. per minute until −40° C. is reached, then accelerates cooling rate. The cryopreservation is performed under protocol SOP-0948A.

### Example 6

### Neural Plasticity and Transplanted NR1 Cells in a Rodent Model of Stroke

The purpose of this study was to investigate the effect of NR1 cells on plasticity by analyzing how NR1 cells affect cortical activity in the peri-infarct area of nude rats after distal cortical stroke.

Brain plasticity, or rewiring, plays a key role in the spontaneous recovery observed after stroke in humans and rodents, with rewiring occurring in the surviving circuits adjacent to the stroke site (i.e., the peri-infarct zone). This plasticity enables healthy brain areas to compensate for the functions of a stroke-damaged area. Transplanted stem cells can enhance brain plasticity after stroke. Therefore, we investigated the effects of NR1 cells on plasticity by studying how NR1 cells affect cortical activity in the peri-infarct area of nude rats.

NR1 Pilot Lot #7 was manufactured at Stanford University using the same procedures utilized for cGMP NR1 production:

### Materials and Methods

Summary of Study Design-High Throughput Electrophysiology Assay. The high-throughput electrophysiology approach used in this study was as follows: One week after undergoing distal cortical stroke, nude rats were transplanted with NR1 cells or vehicle control. Seven days after transplantation, rats were euthanized, brains were collected, live slices made, and a multichannel recording probe was placed in the peri-infarct motor cortex (M1 region) perpendicular to the cortex, such that recording sites spanned all cortical layers. Following activation of cortical circuitry (using a brief electrical stimulation delivered via an electrode), local field potentials, a measure of neuronal population activity, were measured simultaneously in all cortical layers.

In vitro synaptogenesis of retinal ganglion neuronal cells (RGCs). Retinal ganglion neuronal cells (RGCs) were cultured per the standard lab protocols from the Barres lab at Stanford University. NR1 cells were then co-cultured in chambers above the RGCs preventing contact between the cells but allowing the exchange of secreted factors. As a control, astrocytes were co-cultured with RGCs in parallel. After 24 hours, the RGCs were processed and the extent of synaptogenesis was determined by counting the number of cells bodies.

RNA sequencing of brain tissue surrounding the NR1 transplantation site. The gene expression patterns of rat brains that received NR1 and control vehicle were assembled as follows: At 7 days after transplantation, total RNA was extracted from rat brain biopsies and cDNA libraries were generated following standard molecular biology procedures. After preparing cDNA libraries from 7 NR1-treated and 4 control vehicle-treated rats, RNA sequencing was done using a LuminexR Hiseq 2500 high throughput sequencer. Subsequent gene ontology analysis was accomplished using DESeq2 software.

### Results Summary

NR1 cells increase motor circuit activity by releasing individual neurons in the peri-infarct region from inhibitory control. Upon electrical stimulation, both the control and NR1 transplanted groups produced a characteristic response that started in superficial layers near the brain surface, and then propagated to deeper layers. Most notably, NR1 transplantation significantly (P<0.05) increased motor circuit activity in the peri-infarct cortex at Day 7 after transplantation, most prominently in cortical layers 2/3.

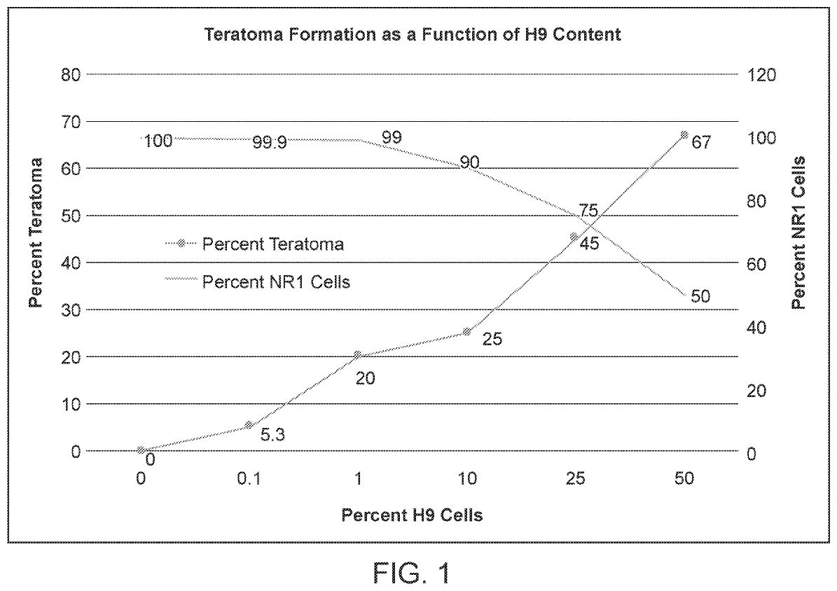

Additional experiments investigated how single neurons within the peri-infarct zone were affected by NR1 treatment by measuring responses from individual pyramidal neurons in layer 2/3 after applying electrical stimulation. Subsequent analysis could reliably estimate the contribution of inhibitory versus excitatory connections to the overall measured response in the neuron. In the NR1 group, recorded inhibitory connections were significantly (P<0.05) reduced in magnitude and delayed in onset, while there were no significant effects of NR1 on excitatory connections (FIG. 1c). Together, these data suggest that NR1 cells increase motor circuit activity by releasing individual neurons in the peri-infarct region from inhibitory control, and show that NR1 cells favorably alter brain activity. NR1-secreted TGFβ3, one of the highest expressed NR1 factors in vivo,5 could be involved in NR1 effects on layer 2/3 plasticity, as TGFβ3 is upregulated specifically in cortical layer 2/3 neurons after stroke.6

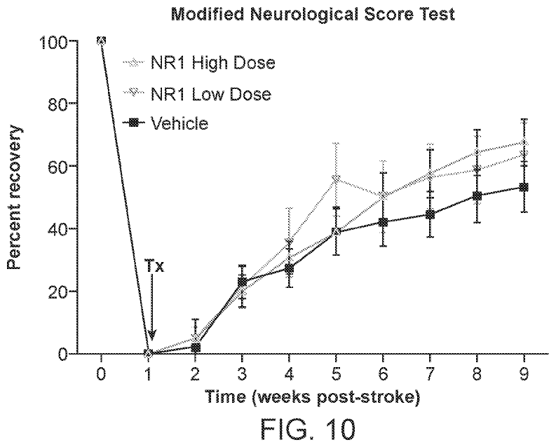

FIG. 13. Neurons in the peri-infarct region are released from inhibitory control by transplanted NR1 cells. Stroke-induced brains were injected with NR1 cells in the peri-infarct area. After 7 days, electrical stimulation of live slices were made and the local field potentials were measured simultaneously in all cortical layers. a) Heat map showing spatiotemporal pattern of motor cortex response to L2 (excitation) and L3 (inhibition) stimulation. b) Maximal excitation in cortical layer 2 across a range of stimulations. Data are mean+SEMs. c) Response from individual neurons in layers 2 and 3 were separated in excitatory (Ge) and inhibitory (gi) components with magnitude of the response in the y-axis and time on the x-axis. Representative traces from each condition are shown.

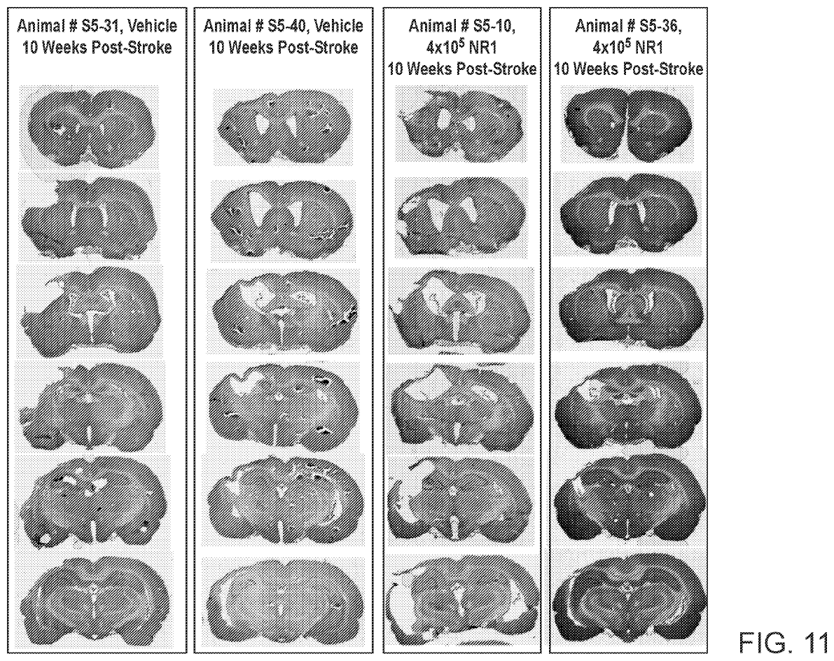

NR1 cells increase synaptogenesis in vitro: In addition to altering plasticity in vivo, NR1 cells can alter plasticity in vitro, in this case by synapse formation. Results from studies done, in collaboration with the Barres lab at Stanford University, indicate that NR1 cells in a non-contact co-culture with retinal ganglion neuronal cells (RGCs), enhanced RGC synaptogenesis to the same extent as astrocytes6 (see FIG. 14). This preliminary result offers a potential potency assay and a way to identify NR1-secreted factors involved in modulating plasticity.

FIG. 14: Synaptogenesis is enhanced in vitro by the addition of NR1 cells. Retinal ganglion neuronal cells (RGCs) were co-cultured under non-contact conditions with either astrocytes or NR1 cells. Synaptogenesis was quantified by counting cell bodies. Significant differences were observed between control astrocytes and astrocytes and NR1 cells (indicating that NR1 cells stimulate synaptogenesis by secretion of factors. Graph=Mean+Standard Error of the Mean (SEM). **P<0.01 by Dunn post-hoc test. ***P=0.003 by Kruskal-Wallis test.

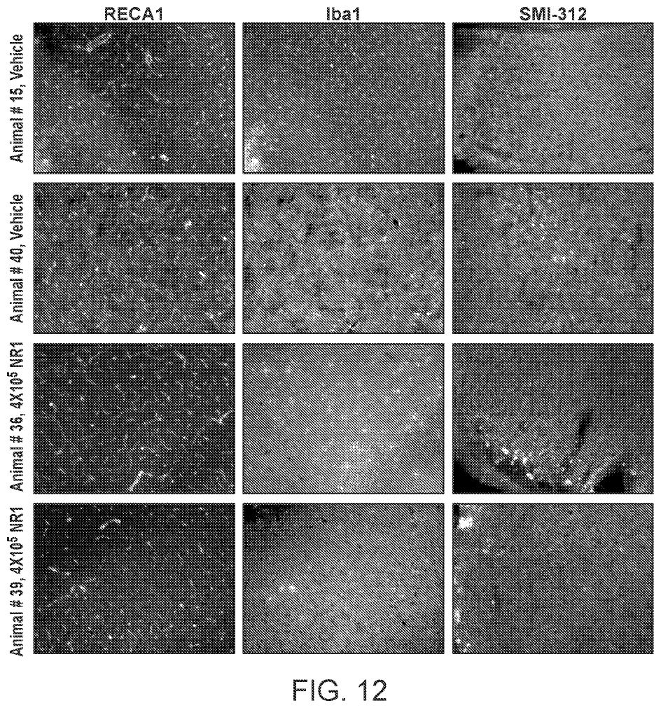

To investigate the role of transplanted NR1 upon reduction of inhibitory circuits, we compared gene expression patterns of rat brains that received NR1 and control vehicle. At 7 days after transplantation, RNA was extracted from rat brain biopsies from the NR1 and control vehicle transplantation site. After preparing cDNA libraries from seven NR1-treated and four control vehicle-treated rats, sequencing by Hiseq 2500 (50 paired-end read) was performed. Gene ontology analysis linked NR1 transplantation with the expression of genes associated with plasticity, suggesting that NR1 cells modulate neuronal activity and plasticity at an early stage, resulting in late functional recovery. A total of 267 genes were differentially expressed when comparing cDNA libraries from the NR1 and control vehicle samples (DEGs, p<0.05, FIG. 15).

FIG. 15: NR1 cells modulate neuronal activity and plasticity at an early stage, resulting in late functional recovery. Stroke-induced adult nude rats were injected with NR1 or control vehicle and sacrificed at 7 days after transplantation. Gene ontology analysis of the cDNA libraries generated from brain biopsies of the transplantation site of the NR1 and control vehicle samples indicated that brains receiving NR1 cells differentially expressed genes involved in stimulating adult neural precursor cell proliferation, neuronal differentiation, gated channels directly involved in the electrophysiological properties of neurons, axon guidance, axonogenosis, and synaptogenesis.

Among the differentially expressed genes in cortical layer 2/3 are: Hapln4/Bral2, Kcnc1/Kv3.1, SCN1a, Slit3 and Slitkr1 (Table 3).7-10 These data suggest that transplanted NR1 modulates neuronal plasticity at an early stage, resulting in late functional recovery.

Conclusions. NR1 cells contribute to the plasticity of peri-infarct regions by releasing individual neurons in the peri-infarct cortex from inhibitory control. NR1 cells modulate plasticity by secreting factors that stimulate synaptogenesis.

### Example 7

### Inflammatory Response in a Rodent Model of Stroke

The purpose of this study is to characterize the inflammatory response triggered by the transplantation of NR1 cells into stroke-damaged nude rat brains.

Stem cell therapy for chronic stroke has shown exciting potential in preclinical studies, with stem cell transplantation conducted weeks to months after stroke significantly improving functional recovery in rodents. Preclinical work support the hypothesis that transplanted cells enhance recovery after stroke by secreting paracrine factors (e.g. VEGF) that modulate the brain's natural repair processes, such as plasticity, inflammation and angiogenesis. Therefore, a possible mechanism of action by which transplanted stem cells induce recovery after stroke is the modulation of the immune response/inflammation. We studied the inflammatory response in stroke brains, as well as the effect of NR1 cells on macrophages in vitro, with the long-term goal of providing a testable model on the role of inflammation in stroke recovery initiated by transplanted stem cells.

### Materials and Methods

Transplantation of NR1 Cells in Stroke Induced Brains. One week after induction of stroke, nude rats were anesthetized and held in a stereotaxic apparatus per Stanford University approved protocols. NR1 cells were prepared according to the standard protocol (NCP-001.01) and diluted in a solution containing Plasmalyte-A and 0.5% HSA at a concentration of 1×10⁵ cells/DL. The NR1 cells were transplanted into the cortex and striatum. For the cortex, two 1.0 μL doses of NR1 (1×10⁵ cells/injection for a total of 2×10⁵ cells injected) were transplanted at the following predetermined coordinates: AP 1.0, ML 1.2, DV −1.5: AP −1.8, ML 2.0, DV −2.0. For the striatum, one 1 uL dose of NR1 (1×10⁵ cells/injection) was transplanted at the following predetermined coordinates: AP −1.8, ML 2.0, DV −5.0. The cells were transplanted at a rate of 0.5 μL/minute. After all of the cells were transplanted, the transplantation needle was held at the coordinate site for an additional 2 minutes before a slow removal. This was done to minimize the occurrence of NR1 cells being “sucked out” of the transplantation site upon removal of the transplantation needle.

Quantitation of M1/M2 macrophage cells. All rat tissues were processed and cells isolated and prepared per Steinberg lab protocols. Macrophage/monocyte cells were sorted as CD11b+Lin− cells, M1 pro-inflammatory/M2 anti-inflammatory macrophages were sorted as Ly6C+/− F4/80+ cells, respectively, using an LSR II sorter at the Stanford Shared FACS facility. The resulting data were analyzed using FlowJo software (FlowJo LLC).

In vitro methods. Bone marrow-derived macrophages (BMDM) were first polarized to either the M1/pro-inflammatory state using lipopolysaccharides, or to the M2/anti-inflammatory state using IL-4. NR1 cells were then either co-cultured in the same well as the BMDMs or in chambers above the macrophages preventing contact between the cells, but allowing the exchange of secreted factors. After 24 hours, the BMDMs were processed and qPCR analysis was done using primer pairs specific for M1/M2 macrophages (M1/pro-inflammatory markers=TNFα. CCL3/MIP1a. M2/anti-inflammatory markers=CD206, Arg1, TGFβ)

### Results Summary

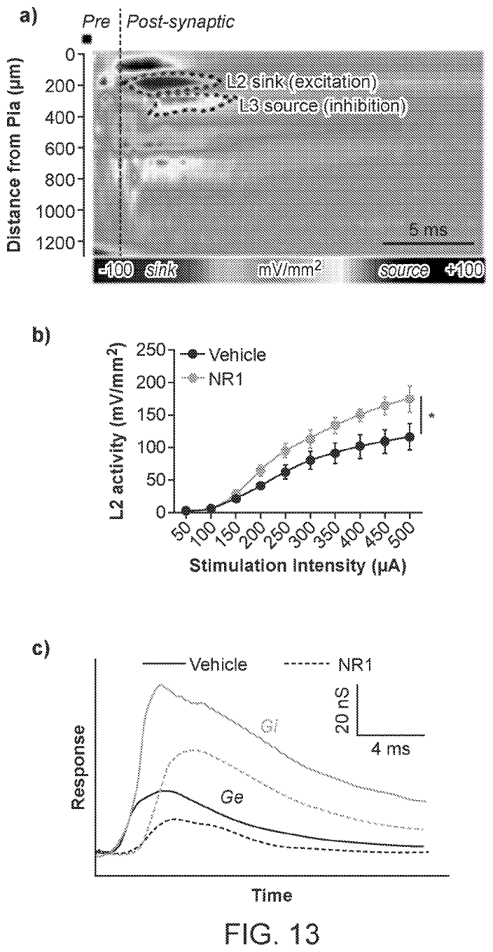

NR1 cells alter the immune response early after transplantation: It has been established that subsets of inflammatory/immune cells contribute to CNS recovery after injury. Modulating the immune response after stroke could therefore be a mechanism by which NR1 cells induce recovery. Using the stroke and transplantation paradigm described above, brain, blood and spleen were collected at different times after transplantation and their immune cell profile analyzed by flow cytometry. Data indicate the immune response is different between NR1- and buffer-transplanted rats (FIG. 16a-c). The stroke-induced rats treated with NR1 exhibited a more variable response compared to the buffer-treated group. The NR1-treated rats also showed a trend for increased numbers of granulocytes and M1- and M2-like monocytes/macrophages in the brain at Day 3 after transplantation. By Day 5, the levels of these sub-populations were declining in the NR1 group. In contrast, the rats treated with buffer alone showed a trend for an increase in both sub-types of monocytes/macrophages at Day 5 (compared to Day 3). This suggests that the temporal response of these populations is different between the treatment groups. The major effect in blood is more “M2” monocytes and Bregs at Day 5 in the NR1 group.

FIG. 16: NR1 injection into a rat stroke-damaged brain alters the immune response. NR1 cells were transplanted into the cortex and striatum of stroke-induced rats per the Stanford-5 efficacy study.12 On Days 3 and 5, the animals were sacrificed and the brain, blood and spleen tissue were processed for FACS in order to analyze the profile of immune modulating cells. a) Profile of M1-like monocyte/macrophages at Days 3 and 5. b) Profile of M2-like monocytes/macrophages at Days 3 and 5. c) Profile of granulocytes at Days 3 and 5.

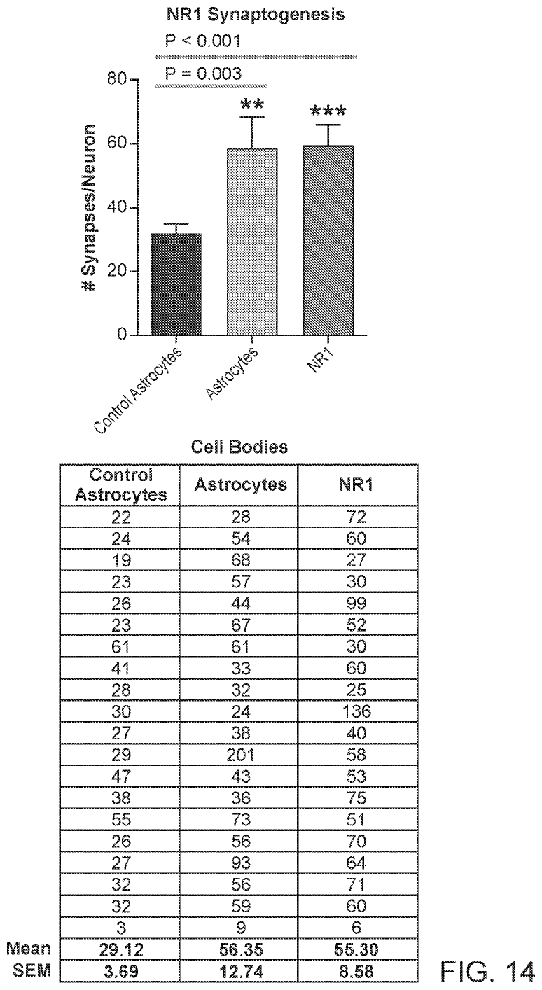

NR1 cells shift the polarization of macrophages from pro-inflammatory to anti-inflammatory in vitro: Macrophages have been classified into a spectrum of functional sub-types (pro/anti-inflammatory), and can switch between sub-types depending on the microenvironment in which they reside. At one end of the spectrum are M1 macrophages that are typically pro-inflammatory; at the other end are M2 macrophages that generally produce anti-inflammatory mediators and are implicated in immunomodulation and tissue repair responses. To determine how NR1 cells affect macrophage polarization, bone marrow-derived macrophages were polarized to pro-inflammatory M1 (by LPS) or anti-inflammatory M2 (by IL-4) sub-types, then incubated with NR1 cells, and harvested 24 hours later for qPCR analysis of M1- or M2-associated markers (FIG. 17a). For M1/LPS-stimulated macrophages, NR1 significantly decreased the expression of the M1 pro-inflammatory factors TNFα and CCL3 and increased expression of the M2 anti-inflammatory cytokine IL-10. For M2/IL-4 stimulated macrophages, NR1 cells increased expression of M2 markers (Arg1, CD206, TGFβ). These data indicate that NR1 cells push macrophages toward the beneficial M2-like state by increasing M2 and decreasing M1 macrophage gene expression

FIG. 17: In vitro polarization of macrophages by NR1 cells. Bone marrow-derived macrophages were first polarized to either the M1/pro-inflammatory state or the M2/anti-inflammatory state by LPS or IL-4, respectively. The M1 and M2 macrophages were then cultured with NR1 cells or control vehicle (Note: The macrophages and NR1 cells were in contact with each other). After 24 hours, the cells were processed and qPCR analysis done using primer pairs specific for M1/M2 macrophages. a) Relative gene expression of M1/pro-inflammatory macrophages in the presence of NR1 cells or control vehicle. b) Relative gene expression of M2/anti-inflammatory macrophages in the presence of NR1 cells or control vehicle. These data indicate that NR1 cells push macrophages toward the beneficial M2-like state by increasing M2 and decreasing M1 macrophage gene expression Note: M1/pro-inflammatory markers=TNFα. CCL3/MIP1α. M2/anti-inflammatory markers=CD206, Arg1, TGFβ

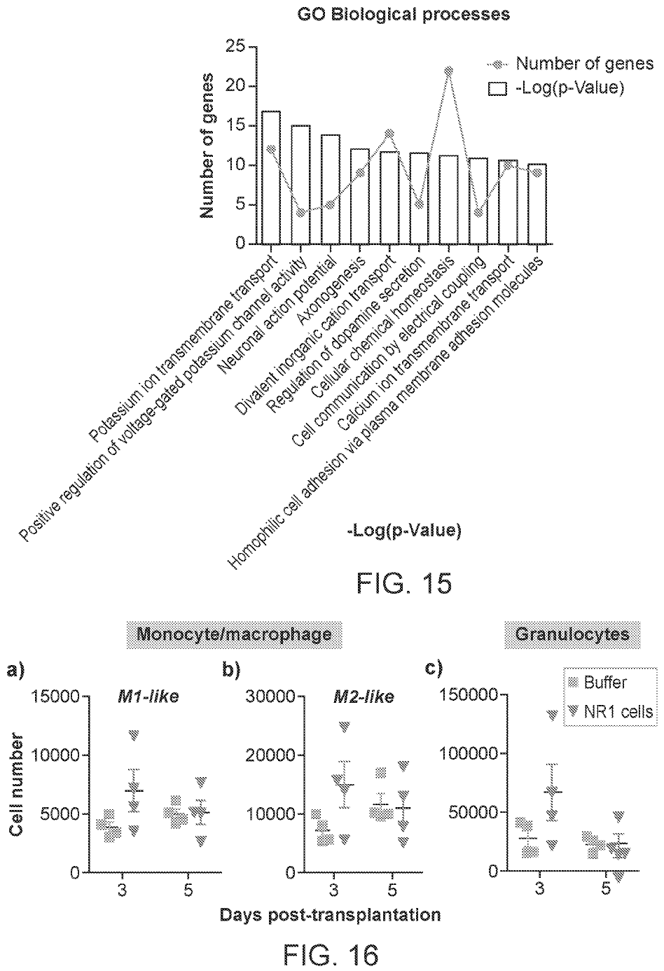

In the previous in vitro experiment, the NR1 cells and macrophages were in contact with one another. In order to determine the effect of factors secreted by NR1 on macrophage polarization, NR1 cells were co-cultured in chambers above the macrophages. As seen in the co-culture experiment in which the NR1 and macrophages were in contact, NR1 cells also polarized the macrophages towards the M2/anti-inflammatory phenotype in this study, indicating that this effect is due to factors secreted by the NR1 cells (FIG. 18). In the M1 polarized macrophages, there is a significant decrease in the expression of the M1 marker TNFα, an increase in the expression of the M2 marker TGFβ, and a significant increase in the M2 marker ARG-1 (FIG. 18a). In the M2-polarized macrophages, there is a significant decrease in the M1 markers MIP1a/CCL3 and TNFα, and a significant increase in the M2 marker ARG-1 (FIG. 18b).

FIG. 18: In vitro polarization of M1/M2 macrophages by NR1-secreted factors. Bone marrow-derived macrophages were first polarized to either the M1 or M2 state as previously described. NR1 cells were then co-cultured in chambers above the macrophages preventing contact between the cells but allowing the exchange of secreted factors. After 24 hours, the macrophages were processed, and qPCR analysis was performed using primer pairs specific for M1 or M2 macrophages. a) Relative gene expression of M1/pro-inflammatory macrophages in the presence of NR1 secreted factors or control vehicle. b) Relative gene expression of M2/anti-inflammatory macrophages in the presence of NR1 cells or control vehicle. b) Relative gene expression of M2/anti-inflammatory macrophages in the presence of NR1 secreted factors or control vehicle. These data indicate that the observed shift to the M2/anti-inflammatory state by NR1 is due to secreted factors. Note: M1/pro-inflammatory markers=TNFα. CCL3/MIP1a. M2/anti-inflammatory markers=CD206, Arg1, TGFβ

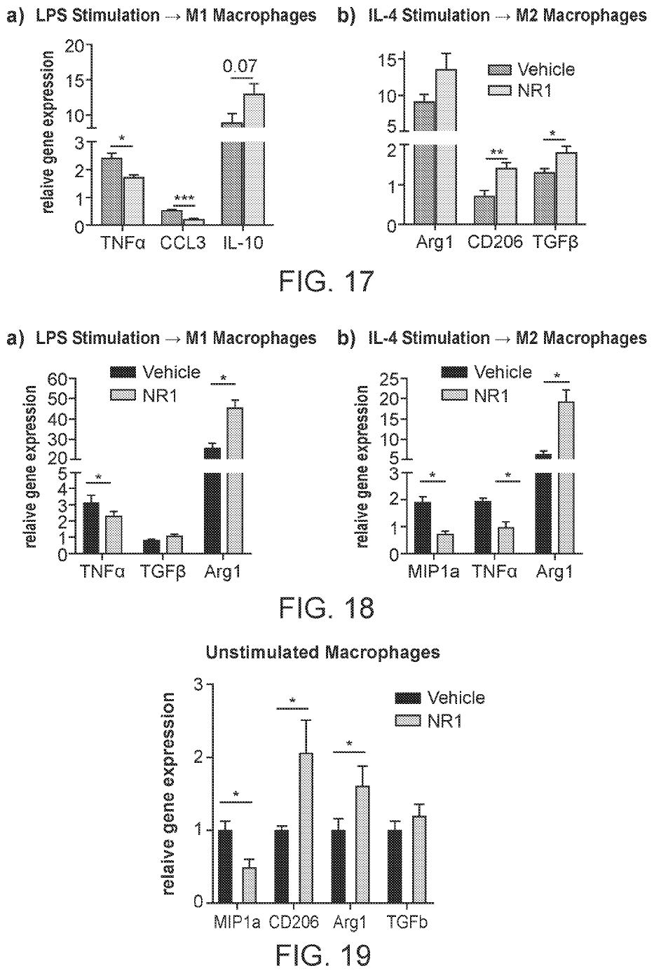

To determine if NR1-secreted factors exert this polarizing effect only on stimulated M1/M2 macrophages, unstimulated macrophages were co-cultured with NR1 cells placed in chambers as previously described. A significant decrease in the M1 marker MIP1a and a significant increase in the M2 markers CD206 and ARG1 indicate that the NR1 secreted factors also push unstimulated macrophages toward an M2 state as indicated by decreasing M1 marker genes and increasing M2 marker gene expression (FIG. 19)

FIG. 19: In vitro polarization of unstimulated macrophages by NR1-secreted factors. BMDM were co-cultured with NR1, where the NR1 cells were placed in chambers above the macrophages preventing contact between the cells but allowing the exchange of secreted factors. After 24 hours, the macrophages were processed, and qPCR analysis was performed using primer pairs specific for M1 or M2 macrophages. The relative decrease the M1 marker (MIP1a) and increase in M2 markers (CD206, ARG1 and TGFβ) indicate that the NR1 secreted factors also push unstimulated macrophages toward an M2 state by decreasing M1 marker genes and increasing M2 marker gene expression. Note: M1/pro-inflammatory markers=TNFα. CCL3/MIP1a. M2/anti-inflammatory markers=CD206, Arg1, TGFβ.

Conclusions. NR1 cells alter the immune response early after transplantation by polarizing macrophages from an M1/pro-inflammatory state to an M2/anti-inflammatory state. In vitro, NR1 cells alter the immune response by secreting factors that push both unstimulated and M1-/M2-stimulated macrophages toward the beneficial M2/anti-inflammatory state by increasing M2- and decreasing M1-macrophage gene expression.

### Example 8

### NR1 Cell Survival Study in Naïve Rodent Brains

The purpose of this study is to determine the survival levels of transplanted NR1 cells into naïve rodent brains at Day 1, 3 and 7 after transplantation.

It has been established that transplantation of neural stem or regenerative cells, including NR1 cells, into the stroke-induced brains of rodents significantly improves functional recovery. As an initial characterization of this process, it was of interest to determine the survival profile of NR1 cells over time after transplantation. Therefore, we undertook a study on the survival rates of transplanted NR1 cells into the brains of naïve nude rats.

### Materials and Methods

Transplantation of NR1 Cells into Naïve Nude Rat Brains. Adult rats were anesthetized and secured in a stereotaxic apparatus per Stanford University-approved protocols. Animal body temperature was measured using a rectal thermometer, and temperature was maintained during the experiment using a homeothermic blanket unit. NR1 cells were prepared according to the standard protocol (NCP-001.01 and diluted in a solution containing Plasma-Lyte A and 0.5% HSA at a concentration of 1×10⁵ cells/μL. The NR1 cells were transplanted into the cortex and striatum. In the cortex, two 1.0 μL doses of NR1 (1×10⁵ cells/injection for a total of 2×10⁵ cells injected) were transplanted at the following predetermined coordinates: AP 1.0, ML 1.2, DV −1.5: AP −1.8, ML 2.0, DV −2.0. In the striatum, one 1 μL dose of NR1 (1×10⁵ cells/injection) was transplanted at the following predetermined coordinates: AP −1.8, ML 2.0, DV −5.0. The cells were transplanted at a rate of 0.5 μl/minute. After all the cells were transplanted, the transplantation needle was held at the coordinate site for an additional 2 minutes before a slow removal. This was done in order to minimize the NR1 cells being “sucked out” of the transplantation site upon removal of the transplantation needle.

Tissue Processing. Rats were perfused with 1×PBS and 4% PFA at Days 1, 3 and 7 after cell transplantation. Whole brains were carefully removed, post-fixed overnight and equilibrated in 30% sucrose. Coronal sections were cut at 30 μm from the beginning of AP+1.2 through the AP −2.0. Serial sections were collected in 24-well plates filled with cryoprotectant. One out of six serial sections, 0.18 mm apart starting at A-P+1.1, was taken per brain, as this encompassed the transplantation site. All of the selected sections were processed using standard immunohistochemistry procedures.

Immunohistochemistry. Mouse anti-HuNu primary antibodies, which will specifically stain human nucleus, were incubated overnight at 4° C. (1:1000×dilution, MAB1281, Millipore, Inc.). The primary antibodies were incubated 2 hours at room temperature with a goat-anti-mouse-biotinylated secondary antibody (1:1000, Vector Laboratories), washed, and then incubated with an avidin-biotin-peroxidase complex (30 min, ABC, Vectastain Elite; Vector Laboratories). To visualize the NR1 cells, 3,3′-diaminobenzidine (DAB) staining was used and only the human NR1 cells were stained a brown color. Sections were then mounted in anterior to posterior order on a glass slide.

Stereological cell counts. An optical fractionator stereological method was used to obtain estimates of the total number of HuNu-positive nuclei using Stereo Investigator software (MBF Bioscience, Williston, VT, USA). The cortex and striatum were counted separately. Briefly, every sixth section extending rostral and caudal from AP+1.2 to AP −2.0 (sections spaced 180 m apart) was sampled such that the measurements spanned a total of 3.2 mm (about 18 sections per animal). The transplanted NR1 cells were delineated using a 2.5×objective and a generated counting grid of 75×75 μm. An unbiased counting frame was placed on the first counting area and systemically moved through all counting areas until the entire delineated area was sampled. Actual counting was performed using a 63×oil objective.

Estimates of the total number of stained NR1 cells were obtained using the following formula: E=kΣN, where E is the estimate of the total number of stained NR1 cells in each animal, ΣN is the sum of n values in all the sections analyzed, and k indicates that every kth section was considered.

### Results Summary

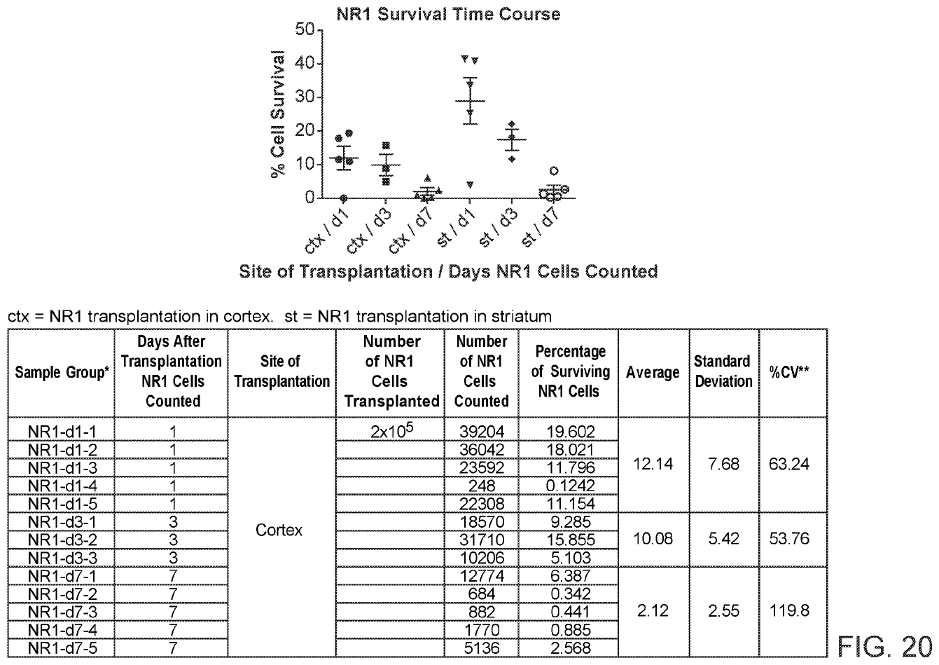

Survival of NR1 Transplanted in the Cortex and Striatum Decreases Significantly from Day 1 to Day 7 after Transplantation: Day 1 after transplantation, the percentages of surviving NR1 cells in the cortex and striatum were 12.1% and 29.2%, respectively. At Day 3, the percentages of surviving NR1 cells in the cortex and striatum were 10.1% and 17.5%, respectively. At Day 7, the percentages of surviving NR1 cells in the cortex/striatum were similar—2.1% and 2.8%, respectively (FIG. 20). No significant differences in the percentages of surviving NR1 cells were seen between Day 1 and Day 3 in either the cortical or striatal transplantations. However, significant differences between Day 1 and Day 7 and Day 3 and Day 7 were seen on both the cortical and striatal transplantations.

FIG. 20: Survival of NR1 cells in cortical/striatal transplantation in naïve nude rats. Rats were transplanted with NR1 cells in either the cortex (two 1 □L injections at 1×10⁵ cells/injection) or striatum (one 1 μL injection at 1×10⁵ cells/injection). They were sacrificed at Days 1, 3 and 7 after transplantation, and 30 μm sections of brain tissue from each time point were stained with DAB and Iba1 (which will only detect human/NR1 nuclei). The number of NR1 nuclei was quantified with a light microscope by counting stained nuclei within each section using an optical fractionator stereological method. Significant differences in the percentage of surviving NR1 cells were seen in both the cortical and striatal transplantations between Days 1 and 7, and Days 3 and 7.

Conclusions. The percentage of surviving NR1 cells did not decrease significantly from Day 1 to Day 3 after transplantation into both the cortex and striatum. The percentage of surviving NR1 cells decreased significantly from Day 1 to Day 7 and Day 3 to Day 7 after transplantation in both cortical and striatal transplantations. The percentage of surviving NR1 cells in both the cortical and striatal transplantations is ˜ 2-3% at Day 7 after transplantation.

### Example 9

### NR1 Secretome Analysis Study

The purpose of this study is to characterize the secretome of NR1 cells.

It has been established that transplantation of NR1 cells into the stroke-induced brains of rodents significantly improves functional recovery in rodents. As an initial characterization of this process, it is necessary to determine the secretome profile of the NR1 cells over time. Therefore, we undertook a study on the secretome of NR1 in vitro and in vivo.

### Materials and Methods

### Summary of Study Design

Transplantation of NR1 GFP-Tagged Cells in Stroke-Induced Brains. One week after induction of stroke, nude rats were anesthetized and held in a stereotaxic apparatus per Stanford University approved protocols. The animal temperature was measured using a rectal thermometer, and temperature was maintained during the experiment using a homeothermic blanket unit. NR1 cells containing a GFP-tagged ribosomal subunit were processed according to the standard protocol and diluted in a solution containing Plasma-Lyte A and 0.5% HSA at a concentration of 1×105 cells/μL.3 The NR1 cells were transplanted into the striatum. One 1 μL dose of NR1 GFP-tagged cells (1×10⁵ cells/injection) was transplanted at the following predetermined coordinates: AP −1.8, ML 2.0, DV −5.0. The cells were transplanted at a rate of 0.5 μL/minute. After all the cells were transplanted, the transplantation needle was held at the coordinate site for an additional 2 minutes before a slow removal. This was done in order to minimize the NR1 cells being “sucked out” of the transplantation site upon removal of the transplantation needle.

TRAP. In vivo secretome: After 2 days the rats were sacrificed and the region of brain tissue receiving the NR1-GFP transplantation was processed for TRAP analysis according to established procedures.3 Briefly, ribosomes and their bound mRNA were isolated via affinity purification with an anti-GFP antibody. The bound mRNA species were further isolated and subjected to RNA sequencing and analysis.

In vitro secretome: NR1 GFP-tagged cells were cultured in NR1 complete media (containing 0.1% and 1% human serum for 24 hours. To analyze the in vitro secretome at the gene level, NR1 cells were isolated and subjected to TRAP/RNA seq analysis as described above.

Protein Analysis. To analyze the in vitro secretome at the protein level, conditioned complete media from NR1 cultures were analyzed by a protein microarray chip containing antibodies specific for known secreted factors and a 63-plex Luminex assay with a bias for immune modulating factors (Immune Monitoring 63-Plex Human ProCartaPlexR Panel, catalog number EPX650-10065-901). Because complete media contains 1% human serum (PHS), there is the potential for serum proteins to mask the NR1 secretome signature. In order to control for this, an NR1 secretome analysis was run using cells grown in low (0.1%) human serum. In order to address the concerns that the low serum conditions could alter the NR1 secretome analysis, a parallel secretome analysis on conditioned media (CM) from NR1 grown in normal (1%) human serum was run.

### Results Summary

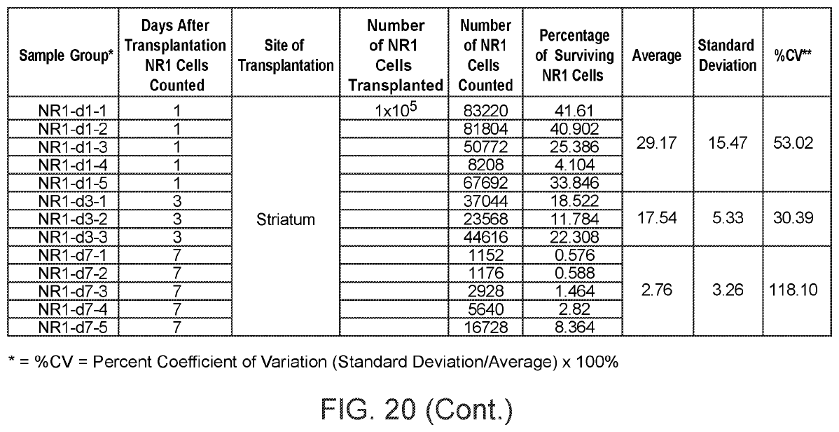

TRAP Analysis of the In Vivo Secretome Indicates That NR1 Cells Express Factors That Modulate Brain Repair and Axon Guidance: In vivo secretome: Using TRAP and RNA sequencing, an analysis of the in vivo secretome of transplanted NR1 cells identified 175 genes encoding for secreted proteins. Gene ontology analysis of these genes indicated that the top 20 enriched processes (FIG. 21) were associated with brain repair, including extracellular matrix (ECM) remodeling (involved in most brain repair mechanisms), processes related to inflammation and axon guidance. These data support the hypothesis that NR1 secreted factors have roles in the modulation of brain repair and axon guidance.

Some of the mostly highly expressed secreted genes by NR1 in vivo include: Col1A1 (collagen type 1 alpha 1 chain), which is involved in ECM organization and can affect leucocyte migration and inflammation4; LGALS1 (galectin 1), which has been shown to shift macrophage polarization to an M2-like (more anti-inflammatory) state in vivo after stroke5; TGFβ3, which regulates ECM formation and has both pro- and anti-inflammatory effects; TIMP1, an inhibitor of MMPs that is involved in ECM degradation. It also has MMP-independent actions, acting as a growth factor with a role in myelin repair. Some of the more highly expressed genes in the NR1 in vivo secretome identified by DAVID Bioinformatics Resources to be involved in plasticity include: COL6A1, COL3A1, MMP2, SPARC, NRG3, and SDF1.

FIG. 21: Gene Ontology of the NR1 in vivo secretome indicates NR1 cells express genes associated with stroke recovery. NR1 cells were transplanted into stroke-induced rats as previously described. At Day 2 after transplantation, the brain tissue was processed and the GFP-tagged ribosomes from the NR1 cells were isolated per established protocol. The isolated RNA transcripts were isolated, sequenced and then analyzed. a) Gene ontology analysis grouping expressed genes based on biological processes. b) Gene ontology analysis grouping expressed genes based on molecular function.

In vitro secretome: TRAP/RNAseq of cultured NR1 cells identified 433 genes in vitro that encoded for secreted factors. Subsequent gene ontology analysis showed the top 5 biological processes involve processes central to NR1-associated stroke recovery: inflammation (162 genes, of which 144 genes code for proteins associated with cytokine activity) and ECM organization (65 genes). Comparing the NR1 in vitro and in vivo RNA sequence-secretome data using DESeq2 software indicated that, of the 175 secreted genes expressed in vivo, are downregulated and 34 are significantly upregulated in response to the stroke microenvironment. Of the genes of interest identified in vivo, NRG3 and TGFb3 were upregulated in response to the stroke environment, while the expression of most of the genes were not significantly different in vitro and in vivo. This suggests that any of these genes of interest could be potential candidates for NR1 potency markers.

In order to characterize the in vitro secretome at the protein level, two assays were performed on CM from NR1 cultures.

Assay 1: CM was tested on a protein microarray chip containing antibodies specific for known secreted factors. Five proteins (FGF18, CSF3, CCL2, FGF7 and FGF17) were found to be potentially involved in NR1 mechanism of action based on their known effects on plasticity, inflammation and angiogenesis. Of these, only CCL2 showed robust expression in the in vitro RNA sequence analysis.

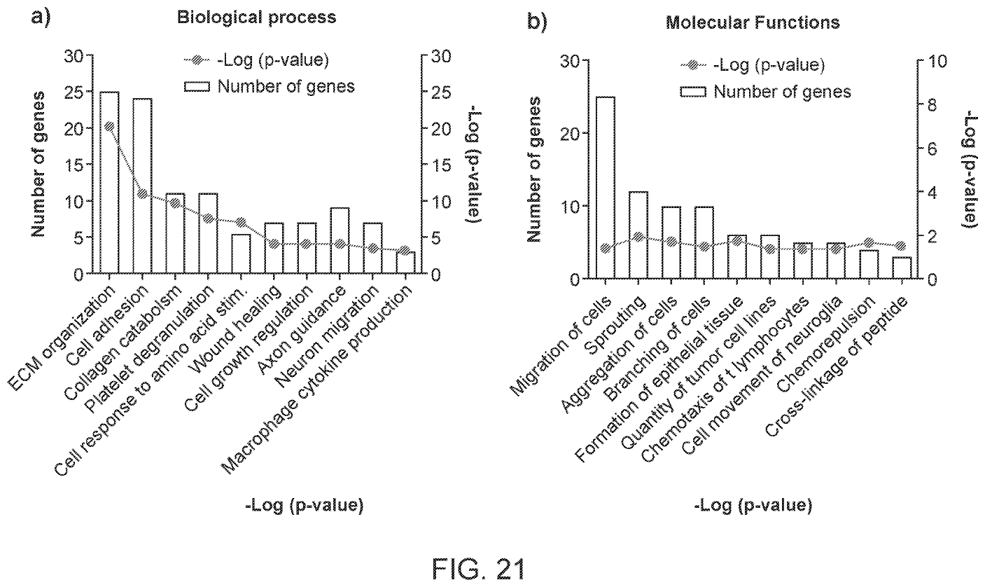

Assay 2: CM was run on a 63-plex Luminex assay (with a bias for immune modulating factors). Of 25 factors identified, the most prominent were: plasminogen activator inhibitor 1 (PAI1/serpine 1)—a modulator of extracellular matrix remodeling; VEGF—an angiogenic factor that also promotes axonal sprouting, neurogenesis and synaptogenesis; MCP1—a chemotactic for immune cells including monocytes and DCs; and SDF1a—an angiogenic factor and chemotactic for lymphocytes and neural progenitors (FIG. 22). These results were observed when the NR1 cells were cultured at either low or normal human serum levels (0.1% and 1%, respectively).

FIG. 22: NR1 secretes proteins linked to stroke recovery. NR1 cells were cultured in either complete media in low (0.1%) human serum or normal human serum (1%). The CM was collected after 24 hours and a 63-plex Luminex assay was run. a). Mean fluorescence intensity (MFI) for both the low (0.1%) and normal (1%) human serum (0.1%) samples from CM and media-only (control) samples. b). The most prominent NR1-secreted factors are involved in chemotaxis of monocytes and dendritic cells (MCP-1), ECM remodeling (PAI1), chemotaxis for lymphocytes and neural progenitors (SDF-1) and angiogenesis (VEGF).

TABLE 10
NR1 In vivo/vitro Secretome Summary

| | | | In vitro | In vitro Secretome at the Protein | |
|---|---|---|---|---|---|
| | | | Secretome | Level (proteins secreted in | |
| | | | at the | conditioned media (CM) | |
| | | | Gene | | CM Tested on 63- |
| | | In vivo | Expression | | plex Luminex |
| | | Secretome | Level | CM Tested | assay (Bias |
| | | (TRAP, | (TRAP/ | on Protein | towards immune |
| GENE | ROLE | RNAseq) | RNAseq) | Microchip | modulating factors) |
| Galectin1/LGALS1 | Known to polarize macrophages to the M2 state. M2 | X* | X* | NA | NA |
| | macrophages produce anti-inflammatory mediators | | | | |
| | and are implicated in in immunomodulation | | | | |
| | and tissue repair responses | | | | |
| TGFb3 | Regulates ECM formation and has | X* | X* (upregulated | NA | X* (TGFb, not |
| | pro/anti-inflammatory effects | | in stroke | | specifically TGFb3, |
| | | | environment)** | | tested but not present |
| | | | | | above background) |
| TIMP1 | Inhibits MMPs that are involved in ECM degradation. | X* | X* | NA | NA |
| | Acts as a growth factor with a role in myelin repair | | | | |
| | (MMP independent action) | | | | |
| SPARC | Involved in cell proliferation, repair | X* | X* | NA | NA |
| | of tissue damage, and morphogenic processes such | | | | |
| | as re-modeling of the extracellular matrix. | | | | |
| NRC3 | Involved in neural plasticity | X* | X* (upregulated | NA | NA |
| | | | in stroke | | |
| | | | environment)** | | |
| SDF1 (a)/CXCL-12 | An angiogenic factor. Also a chemotactic factor for | X* | X* | NA | X* |
| | lymphocytes and neural progenitors | | | | |
| CSF3/G-CSF | Role in neural plasticity inflammation and angiogenesis | ND | ND | X* | X* |
| VEGF | An angiogenic factor. Promotes axonal sprouting, | X* | X* | NA | X* |
| | neurogenesis, synaptogenesis | | | | |
| PAI1/serpine 1 | modulates extracellular matrix remodeling | X* | X* | NA | X* |
| Col1A1 | Involved in wound healing during | X* | X* | NA | NA |
| | spinal cord/neuron damage | | | | |
| Col6A1 | Associated with the diassembly of ECM | X* | X* | NA | NA |
| | constituents/tissue remodeling | | | | |
| Col3A1 | Involved in maintaining soft connective tissue | X* | X* | NA | NA |
| MMP2 | Involved in the neurogenic response of adult neural | X* | X* | NA | NA |
| | stem/progenitor cells | | | | |
| NRG3 | A critical mediator in the assembly | X* | X* | NA | NA |
| | of cortical inhibitory circuits | | | | |
| FGF18 | Regulates proliferation of neural stem cell/progenitors | ND | ND | X* | NA |
| CCL2 | Chemotactic for immune cells including | ND | ND | X* | NA |
| | microglia and monocytes | | | | |
| FGF7 | FGF7 promotes inhibitory synapse formation | ND | ND | X* | NA |
| FGF17 | Involved in neocortical reorganization | ND | ND | X* | NA |
| MCP-1 | Chemotactic for immune cells including monocytes | ND | ND | NA | X* |

*= Gene detected in the assay indicated
ND = Not Decected.
NA = Not Applicable (candidate gene/protein was not present in the assay)

### Conclusion

The in viva/in vitro secretomes of NR1 cells indicate that NR1 cells secrete factors that modulate a variety of factors that are in involved in processes associated with late functional recovery.

### Example 10

### Effect of Microglia Depletion on Transplanted NR1 Survival in Naïve NSG Mice Brains

The purpose of this study is to determine if depleting microglia, by use of PLX5622 before and after NR1 transplantation into naïve NSG mice brains, will increase NR1 survival.

NR1 cells have a very short survival period following transplantation into the rodent brain, with only −2% of the cells surviving at 1 week after transplantation. A host immune response to the cells could have a deleterious effect on NR1 survival. As microglia are brain resident macrophages, and likely first responders to the transplanted NR1 cells, we studied the effects of microglia depletion, by PLX5622, on transplanted NR1 survival in naïve NSG mice brains.

Summary of Study Design. Overview of the experiments
a) Testing microglia depletion by PLX5622 in NSG mice
b) Testing NR1 survival following microglia depletion with PLX5622
i. Study 1: striatal plus cortical NR1 transplantation
ii. Study 2: only striatal NR1 transplantation

Microglia depletion in adult NSG mice by exposure to PLX5622. Adult NOD scid gamma mice (NSG) mice, housed 5 mice/cage, were fed a diet of standard chow or standard chow containing 1200 mg of PLX5622 per kg, which is a drug that targets CSF1-/microglia cells.1 The chemical PLX5622 was provided by Plexxikon (Berkeley, CA) and formulated in AIN-76A standard chow by Research Diets (New Brunswick, NJ). NSG mice were fed, ad libitum, the control or PLX5622 chow for 1 or 3 weeks. After 3 weeks of treatment, mice received NR1 cell transplants into the brain. Following transplantation, mice were maintained on the standard chow or PLX5622 chow for an additional 7 days. During treatment, mice were monitored at feeding intervals to assure they were healthy. The food was stored in sterile bags until supplied to the mice. Animals were housed in the Steinberg lab mouse room (SCORE, P059N) in a cage labeled with a pink card with specific information, including name of chemical (PLX5622), dosage, date/time animal was dosed, expected end date of treatment, and a yellow hazardous drug sticker. Cages were checked twice per week to ensure the level of food and proper labeling.

Surgical procedure for the transplantation of NR1 cells into naïve NSG mice. Adult naïve NSG mice were prepared for NR1 transplantation surgery following surgical procedures established in the Steinberg laboratory.2 NR1 cells were prepared, using PCP-001.01,2 at a final concentration of 1×10⁵ cells/μl, and administered as an intracerebral injection in the proposed clinical formulation (Plasma-Lyte A plus 0.5% HSA). For Study 1, cells were injected into the striatum (2 ul/injection site, i.e., 2×10⁵ cells/deposit; 4×10⁵ cells total) and into the cortex (1 ul/injection site, i.e., 1×10¹ cells/deposit, 2×10¹ cells total) with the following coordinates:
AP: −0.34, ML: +2.5, DV: −2.5 (striatum)
AP: −0.34, ML: +2.5, DV: −0.95 (cortex)
AP: +0.96, ML: +1.5, DV: −2.25 (striatum)
AP: +0.96, ML: +1.5, DV: −0.75 (cortex)

For Study 2, cells were only injected into the striatum to reduce the potential loss of cells from ‘leaking out’ of the cortex. The same striatal coordinates and injection volumes were used as above. NR1 administration was performed using a Hamilton micro-syringe modeled after the intended clinical delivery. A separate group of animals was injected with vehicle control (Plasma-Lyte A plus 0.5% HSA) using the same injection method. The cells were transplanted at a rate of 0.5 μL/minute. After all the cells were transplanted, the transplantation needle was held at the coordinate site for an additional 2 minutes before a slow removal. This was done in order to minimize the NR1 cells being “sucked out” of the transplantation site upon removal of the transplantation needle. The general health of the animals was monitored weekly by clinical observations and recording of body weights. After completion of in-life assessments, brain tissue was collected at the designated post-treatment time points for subsequent FACS analysis.

Quantitation of microglia cells. All mouse tissues were processed and cells isolated and prepared per Steinberg lab protocols. Microglial cells were defined as CD45int/CD11b+/Lin−, with ‘Lin’ containing the lineage markers B220, CD3, CD11c, CD49b, CD90.2, Ly6G, NK1.1, Ter119. Microglia cells were quantified using an LSR II sorter at the Stanford Shared FACS facility, and the resulting data analyzed using FlowJo software (FlowJo LLC).

Tissue processing for the quantitation of surviving NR1 cells. Mouse brains were perfused with 1×PBS and 4% PFA 7 days after cell transplantation. Whole brains were carefully removed, post-fixed overnight and equilibrated in 30% sucrose. Coronal sections were cut at 30 am from the beginning of AP+1.2 through the AP −2.0. Serial 30 μM thick sections were collected in 24-well plates filled with cryoprotectant. For Study 1, one out of six serial sections, 0.18 mm apart, was taken throughout the entire brain; for Study 2, one out of four serial sections, 0.12 mm apart, was taken. All selected sections were processed using standard immunohistochemistry procedures.

Immunohistochemistry of brain sections. Brain sections were incubated overnight at 4° C. with an anti-human antibody (anti HuNu at a 1:1000×dilution, MAB1281, Millipore, Inc.). Secondary antibodies were incubated 2 hours at room temperature with biotinylated secondary antibody (1:1000, Vector Laboratories), washed, and incubated with an avidin-biotin-peroxidase complex (30 min, ABC, Vectastain Elite; Vector Laboratories). The 3,3′-diaminobenzidine (DAB) staining was used and the human stem cell was stained a brown color. Sections were then mounted in anterior-posterior order on a glass slide.

NR1 cell counts. NR1 cells were counted using Stereo Investigator software (MBF Bioscience, Williston, VT, USA). Due to small numbers of surviving cells per brain slice, all cells in the stained slices were counted rather than using a stereological counting method. Counting was performed using a 63×oil objective in a blinded manner. Briefly, every sixth section (Study 1) or fourth section (Study 2) extending throughout the entire brain was sampled. Total NR1 cell counts were calculated as either 6×number of cells counted in 1:6 series, or 4×number of cells counted in 1:4 series.

### Results

Mice Fed PLX5622 Chow Had Decreased Microglia Levels. NSG mice fed PLX5622 showed a significant decrease in microglia levels in the brain, with greater microglia depletion being observed after 3 weeks versus 1 week of PLX5622 treatment. In mice fed PLX5622 for 1 week prior to NR1 transplantation, microglial levels were 61% of the control group, i.e., decreased by 40%. In mice fed PLX5622 for 3 weeks prior to NR1 transplantation, microglial levels were 25% of the control group, i.e., decreased by 75%. Based on these data, the 3 week pretreatment with PLX5622 was chosen for subsequent NR1 survival studies.

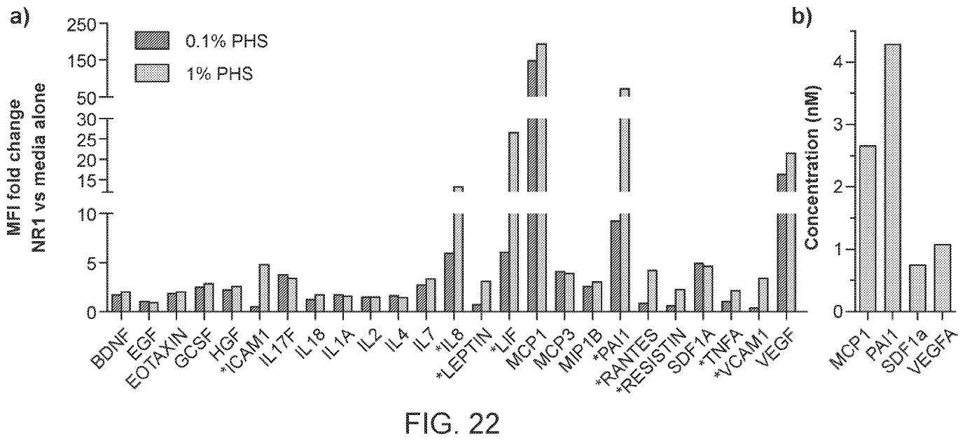

FIG. 23: PLX5622 significantly depletes brain microglia. NSG mice were fed PLX5622 or control chow for either a) 1 week or b) 3 weeks prior to sacrifice. Mean±SD. *p<0.05 by two-tailed T-test; ***p<0.001 by two-tailed t-test with Welch correction.

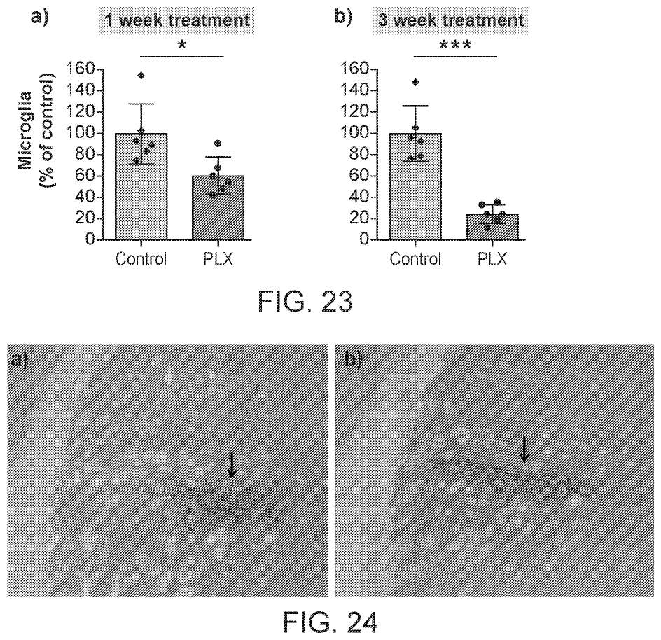

Effect of microglia depletion on NR1 survival. NSG mice were fed PLX5622 chow or control chow for 3 weeks prior to NR1 intracerebral transplantation and for 1 week following NR1 transplantation, after which the animals were sacrificed. NR1 survival was observed in both control and PLX5622 groups (FIG. 24).

FIG. 24: NR1 survival in NSG mouse brain. Naïve NSG mice were fed control/PLX5622 mouse chow as described and sacrificed 7 days after NR1 transplantation. In order to detect surviving NR1 in the mouse striata, 30 μm sections of brain tissue from control and PLX5622 samples were stained with HuNu, which will only detect human/NR1 nuclei. a) Representative image of a 30 μm brain section from naïve NSG mice fed control mouse chow showing stained NR1 cells (red arrows). b) Corresponding stained section from naïve NSG mice fed PLX5622 mouse chow.

Study 1: In this study NR1 cells were transplanted into both striatum and cortex using the same needle track. Overall, there was a trend for increased NR1 survival in the PLX5622-treated animals but this was not statistically significant, and NR1 survival remained very low at 2.37%.

Study 2: In this study, NR1 cells were transplanted only into the striatum to minimize the potential for leakage of cells from the brain along the needle track. Quantification of NR1 survival showed a slight trend for increased NR1 survival in the PLX5622-treated animals.

Conclusion. PLX5622 significantly decreases the number of microglia cells in brain. Depleting microglia numbers in NSG mice only marginally increases the percentage of surviving NR1 cells at 7 days after transplantation.

### Example 11

### T2-FLAIR Signal and TSPO-PET as a Predictor of NR1-Induced Functional Recovery in Chronic Stroke

To investigate the immunomodulatory effects of NR1 transplantation in chronic stroke using a T2-FLAIR MRI signal in a rat model as a clinical predictor of NR1-induced functional recovery in stroke patients.

Achieving a ‘cure’ for the chronic disabilities resulting from stroke became one step closer to reality after two recent stem cell clinical trials. In these early phase trials, stem cells were injected into the brains of patients who were >6 months to years after their stroke (i.e., chronic time point), when all natural recovery has plateaued. With stem cell treatment, however, most patients experienced clinically meaningful improvement. Most notably, two patients in the SanBio trial exhibited remarkable recovery regaining significant limb use.

These results raise fundamental questions about what occurred in these patients' brains to trigger such recovery. A potential clue is the surprising discovery in the SanBio trial that the extent of motor recovery at 6, 12 and 24 months after transplant positively correlated with the size of a brain MRI signal-specifically a T2-FLAIR signal located primarily in the premotor cortex. This implies that by identifying the stem cell-induced brain changes causing the FLAIR signal we can start to dissect the cellular and molecular mechanisms involved in stroke recovery.

Study Objective. To investigate the brain changes underlying the stem cell-induced FLAIR signal, we reproduced this phenomenon in a rat model of chronic subcortical stroke with stem cell transplantation at the chronic phase of stroke.

As the T2-MRI FLAIR signal is often associated with inflammation we conducted a preliminary immunohistochemistry analysis of the FLAIR lesion in rats, which was consistent with inflammation. Given the correlation between the FLAIR signal and recovery in the SanBio trial, this implies brain inflammation could be a useful biomarker for stroke recovery at the chronic stage.

To investigate this with greater sensitivity and specificity, we established a second non-invasive imaging modality to monitor brain inflammation in rats—PET imaging of the translocator protein 18 kDa (TSPO) i.e., TSPO-PET. In the healthy brain TSPO levels are low, but under neuroinflammatory conditions TSPO is markedly upregulated. The predominant cell types expressing TSPO are activated immune cells including brain resident immune cells, microglia and astrocytes, and infiltrating myeloid cells, such as monocytes/macrophages, neutrophils and dendritic cells. TSPO-PET radioligands therefore serve as a useful index of neuroinflammation, and increased TSPO-PET signals have been observed in stroke patients, both in the stroke lesion and in remote brain areas, in studies ranging from 5 days to 24 months after stroke.7-10

### Study Design/Methods

The T2-FLAIR studies were performed in Sprague Dawley (SD) rats using a suture model of subcortical stroke. Stem cell transplantation was performed at the chronic phase (1 month after stroke) to best mimic the parameters of the SanBio clinical trial. FLAIR lesion analysis conducted by an experimenter blinded to the treatment groups. Immunohistochemical analysis of the needle tract region associated with the FLAIR lesion was performed according to the Steinberg lab IHC protocol.

The TSPO-PET studies were performed in nude rats using the dMCAO model of cortical stroke with cortical transplantation of NR1 cells at the sub-acute phase of stroke (7 days after stroke).

### Results

T2-MRI FLAIR. The rodent FLAIR signal mimics the human counterpart in that it appears transiently along the needle track in the first week after transplantation but is most prevalent in the motor cortex despite cells being transplanted subcortically. It is diffusion weighted imaging (DWI)-negative, indicating that the FLAIR signal is not due to a new acute ischemic infarct (DWI is more sensitive to early changes after a stroke than more traditional MRI measurements). Most notably, we have not observed a FLAIR signal after vehicle injection, confirming the FLAIR signal is a cell-specific phenomenon. These data were consistently found over three separate experiments.

Immunohistochemical Analysis. Immunohistochemical analysis of the needle track region associated with the FLAIR leison demonstrates qualitative differences in the inflammatory response between NR1- and vehicle-treated animals. At 1d after transplantation, cell-treated animals appear to have more Iba1-positive macrophages/microglia, particularly exhibiting more activated ameboid cells at the border of the needle track. Astrocyte (GFAP) number does not appear grossly different, however astrocytes in the cell animals have thicker primary processes and less branching than in the vehicle animals, indicative of a more activated state, although A1 versus A2 polarization cannot be ascertained by morphology. These data establish a precedent for NR1s modifying the inflammatory response in the FLAIR lesion.

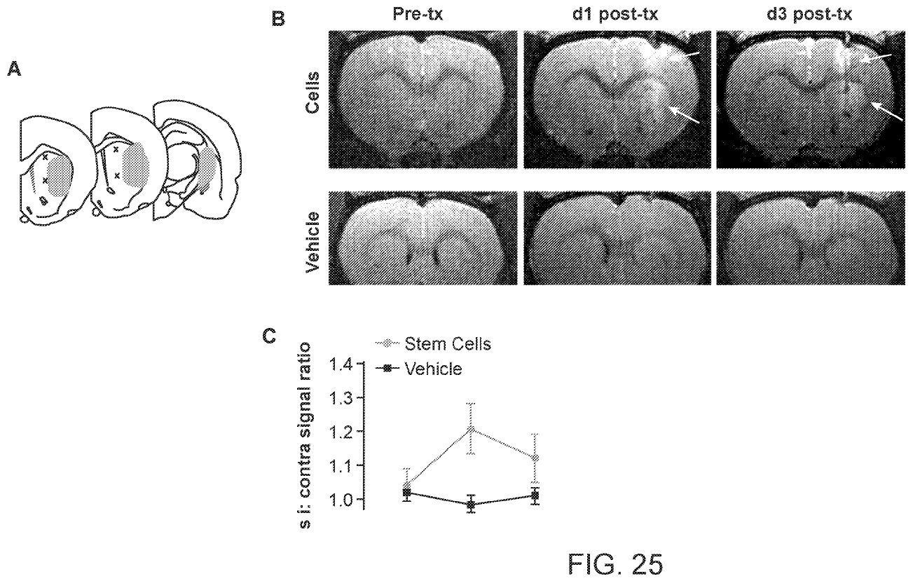

FIG. 25. T2-FLAIR signal in rat cortex before and after NR1 transplantation. (A) Schematic of stroke location (grey) and transplantation coordinates. (B) T2-FLAIR MR images in the needle track region from rats before and after transplantation (tx) (red arrows point to FLAIR signal). (C) Quantitative analysis of the FLAIR signal. Mean±SEM; n=5/group from separate experiments. No statistically significant differences were observed with this small n.

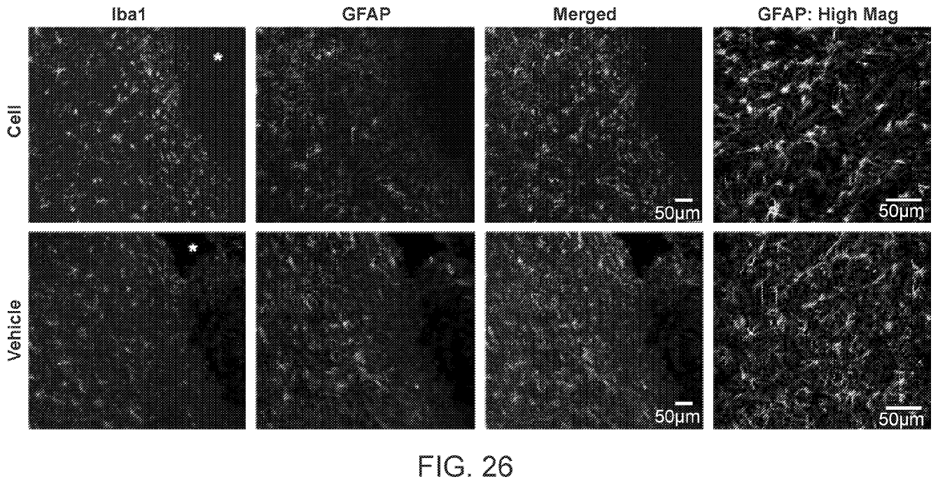

FIG. 26. Immunohistochemical analysis of the needle track region associated with the FLAIR lesion. Analysis of SD rats with cortical stroke injury at 1 day post-treatment with NR1 or vehicle. GFAP indicates astrocytes. *indicates needle track.

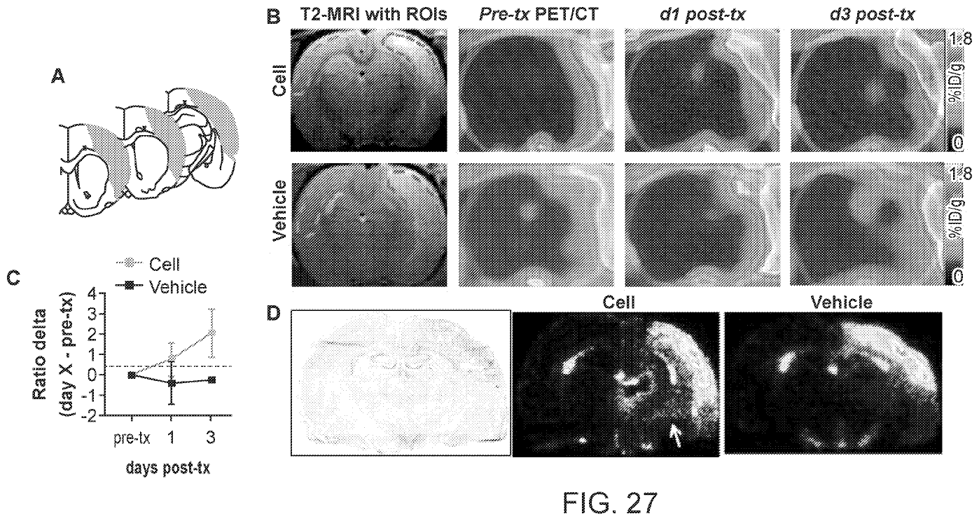

FIG. 27. NR1 transplantation increases the post-stroke TSPO-PET signal. Nude rats with cortical stroke injury and cortical transplantation of NR1 in the subacute phase of stroke (Day 7 after stroke) (A) exhibited an increased cortical TSPO signal over the first 3 days after transplantation in NR1, but not in vehicle-treated, rats (B, C: mean±sem; n=6). The increased TSPO signal at Day 3 after transplantation was in the transplantation vicinity (B), the infarct core (B: red dotted region denotes core), the basal ganglia (arrow in D: autoradiographs and cresyl violet staining), and the hippocampus (not shown).

Our TSPO-PET data are consistent with the FLAIR data, showing higher immune cell activity in the NR1 versus vehicle-treated rats. This was observed both near the transplantation site and also in more distant, but connected brain regions, implying widespread immunomodulatory effects of the NR1s. Remote inflammation is considered detrimental. However, in chronic stroke patients, a TSPI-PET signal in remote brain regions correlated with functional recovery. Thus, remote inflammation could be beneficial at this stage of stroke.

This work, in conjunction with our in vivo secretome analysis and SanBio clinical trial, strongly implicates immunomodulation as a major mechanism of action of stem cells in stroke recovery.

### Example 12

### NR1 Toxicology Summary

A GLP toxicology study and three pilot toxicology studies conducted with the NR1 drug provided consistent results showing the safety and tolerability of the NR1 cell product injected intracerebrally into the parenchyma of the brain. In addition, a GLP biodistribution study, two pharmacology studies and 7 other investigative studies had similar outcomes that demonstrated that NR1 drug product was well tolerated and was not associated with the formation of teratomas in the brain or peripheral tissues at NR1 intracerebral doses as high as 2×10⁶ cells per animal.

The pivotal single-dose GLP toxicology study incorporated a 180-day long treatment-free follow-up period to allow a significant duration of time for the detection of slow-forming teratomas potentially related to treatment with NR1 drug product. The cell injection medium used in the study contained Matrigel, which is a complex protein mixture derived from mouse Engelbreth-Holm-Swarm sarcoma cells that is reported to contain murine growth factors. Matrigel is known to promote survival of stem cell-derived neural progenitors in somatic tissues. The objective of including Matrigel was to fully support cell growth, both of NR1 cells and any contaminating stem cells create a “worst-case-scenario” and thereby increase the likelihood of teratoma development and growth by supplying the optimal growth conditions, thus imposing the highest possible bar for the assessment of the safety of NR1. Even in the presence of the growth-supporting matrix including 40% Matrigel, no teratomas were detected in any study conducted with NR1 and, specifically, none were detected in the GLP toxicology study in which 100 male and female athymic nude rats received up to 2×10⁶ NR1 cells per animal and were followed for up to 6 months after transplantation.

The athymic nude rat model used in the study was capable of supporting the growth of stem cell-derived teratomas under the conditions of the study. Treatment groups that contained both NR1 cells with H9 hESC spiked in at levels ranging from 2×10³ to 1×10⁶ H9 cells per animal (0.1% to 50% of the total NR1 dose, respectively) did result in the formation of teratomas. Teratomas occurred in a dose-dependent manner in the H9-spiked groups, ranging from 67% (12/18 animals) in Group 4 (50% H9 spike) to 5.3% (1/19 animals) in Group 8 (0.1% H9 spike). None of the groups that were treated with unspiked NR1 drug product alone formed teratomas. Microscopically the teratomas present in treatment groups receiving H9-spiked NR1 at levels of 10% and greater consisted mostly of tissues from endoderm, mesoderm and ectoderm, including a mixture of tissues such as skin, glands, cartilage and/or bone formation, as well as nervous tissue. Teratomas in these groups tended to be large. Microscopic, but not macroscopic teratomas, were seen in groups receiving H9-spikes of 1% and 0.1% (Groups 7 and 8), and these teratomas consisted primarily of smaller formations of haphazard-appearing nervous tissue with choroid plexus-like formation, ependymal epithelium, and/or bulging areas of near-normal looking white matter. They did not contain tissues derived from all three germ layers. None of the groups that were treated with NR1 drug product only, formed teratomas. No evidence of teratoma formation outside of the brain or distant from the injection site was observed in any H9-spiked or NR1 IC or IM-treated group.

The role of Matrigel in promoting the growth of teratomas is not entirely understood, but it is clear that the matrix also supports the growth and persistence of NR1, as shown in studies in which NR1 cell persistence was compared in the presence and absence of Matrigel in the human cell injection medium. In all nonclinical studies, NR1 survival was always improved when Matrigel was in the cell injection medium. However, even in the presence of 40% Matrigel, treatment with doses up to 2×10⁶ cells of the cGMP NR1 drug product alone (which has less than 0.01% residual H9 cells) did not result in toxicity or the formation of teratomas or tumors in adult athymic nude rats over the 6-month duration of the toxicology study, demonstrating its safety over long-term follow-up.

TABLE 11

| Type of | Species/Strain | Test | Test Article and | GLP | Testing |
|---|---|---|---|---|---|
| Study | No. per groups | Article | Doses, Route | Compliance | Facility |
| Six-Month | Adult athymic nude rats | NR1 | Group 1: Plasmalyte-A [symbol] 0. [symbol] % | GLP | MP [symbol] |
| Toxicology Study | (C [symbol] :NIH-F [symbol] ) | (CoH batch | HSA with 40% Matrigel, IC | | |
| of NR1 NPCs | n = 5-25 per sex per group | nos. | Group 2: 4e5 NR1, IC | | |
| Administered | | 1299-128-0002, | Group 3: 2e6 NR1, IC | | |
| Intra [symbol] | | 1299-128-0003, | Group 4: 2e6 total dose (50% | | |
| in the Brain or | | 1299-128-0004) | NR1:50% H9), IC | | |
| Intramuscularly in | | H9 | Group 5: 2e6 total dose (75% | | |
| the Athymic Node | | (Stanford Lot | NR1:25% H9), IC | | |
| Rat | | no. P30) | Group 6: 2e6 total dose (90% | | |
| | | | NR1:10% H9), IC | | |
| | | | Group 7: 2e6 total dose (99% | | |
| | | | NR1:1% H9), IC | | |
| | | | Group 8: 2e6 total dose (99.9% | | |
| | | | NR1:0.1% H9), IC | | |
| | | | Group 9: 1e6 H9, IC | | |
| | | | Group 10: 2e3 H9, IC | | |
| | | | Group 11: 1e7 NR1, IM | | |
| | | | Group 12: 1e7 total dose (50% | | |
| | | | NR1:50% H9), IM | | |
| | | | Group 13: 1e7 total dose | | |
| | | | (99.9% NR1:0.1% H9), IM | | |
| Pilot study of NR1 | Adult athymic nude rats | NR1 | Part 1: | Non-GLP | MP [symbol] |
| neural progenitor | (C [symbol] :NIH-F [symbol] ) | (Stanford pilot | Medium: Plasmalyte-A-HSA | | |
| cell persistence in | n = 5-6 females per group | lot #1) | with or without 40% Matrigel | | |
| the brain in three | Neonatal a [symbol] nude rats | | Group 1: Adult nude rats; NR1 | | |
| animal models | (C [symbol] :NIH-F [symbol] ) | | in Matrigel; 2e6 NR1, IC | | |
| | n = 6 females/group | | Group 2: Ne [symbol] nude rats; | | |
| | Adult mice, NSG | | NR1 in Matrigel; 1e6, IC | | |
| | (NOD.Cg.P [symbol] ) | | Group 3: NSG mice; NR1 in | | |
| | n = 6 females/group | | Matrigel; 1e6 NR1, IC | | |
| | | | Part 2: | | |
| | | | Group 4: Adult nude rats; NR1 | | |
| | | | in Matrgiel; 2e6 NR1, IC | | |
| | | | Group 5: Adult nude rats; 2e6 | | |
| | | | NR1 without Matrigel, IC | | |
| 60-Day pilot study | Adult athymic nude rats | NR1 | Group 1: 2e6 NR1 in | Non-GLP | MP [symbol] |
| of NR1 cell in nude | (C [symbol] :NIH-F [symbol] ) | (Stanford pilot | Plasmalyte-HSA, IC | | |
| rats; cell | n = 3-20 females/group | lot #1) | Group 2: 2e6 NR1 in | | |
| persistence, generak | | | Plasmalyte-HSA + 40% | | |
| safety and potential | | | Matrigel, IC | | |
| for tumorigenicity | | | Group 3: Plasmalyte-HSA | | |
| and [symbol] mation of | | | control, IC | | |
| ectopic tissue | | | Group 4: PLasmalyte-HSA + | | |
| | | | 40% Matrigel control, IC | | |
| | | | Group 5: 2e6 NR1 in | | |
| | | | Plasmalyte-HSA, IC | | |
| | | | Group 6: 2e6 NR1 in | | |
| | | | Plasmalyte-HSA + 40% | | |
| | | | Matrigel, IC | | |
| Pilot NR1 cell | Adult athmic nude rats | NR1 | Group 1-6: NR1 in | Non-GLP | STAN [symbol] |
| survival study in | C [symbol] NIH-F [symbol] ) | (CoH batch no. | DMEM/F12; 1e6, IC | | |
| nude rats with and | n = 24 males/group | 1299-128-0002) | Group 7: H9 in DMEM/F12; | | |
| without Matrigel | | H9 | 1e6, IC | | |
| | | (Stanford pilot | Group 8-13: NR1 in | | |
| | | lot) | DMEM/F12 + 30% Matrigel; | | |
| | | | 1e6. IC | | |
| | | | Group 14: H9 in DMEM/F12 + | | |
| | | | 30% Matrigel; 1e6, IC | | |

IC: Intracerebral via intrapa [symbol] injection into the cortex and st [symbol] of both hemispheres of the brain
IM: Intramuscular into the bicep [symbol] fem [symbol] of the left [symbol] [symbol]
MPI Research ( [symbol] Charles River Laboratory, Matta [symbol] ), Matta [symbol] , MI [symbol]
Stanford University, Stanford

[symbol]

| | | | | | |
|---|---|---|---|---|---|
| | Test Article Dose [symbol] , | Sex and | Obs. Max | Approx | |
| Species | cells/animal | No. per | Nonlethal | Lethal | |
| (Strain) | (% H9 [symbol] pike), Route | Group | Dose [symbol] | Dose [symbol] | Noteworthy Findings |
| Adult athymic nude rats | Group 1: Plasmalyte-A + 0.5% | 5-25 | 2 × 10⁶ | Not | No NR1 drug product-related early deaths, |
| (C [symbol] NIH-F [symbol] ) | HSA with 40% Matrigel | per sex | NR1 cells | determined | clinical signs of toxicity, food consumption or |
| | Group 2: 4e5 NR1 | per | | | body weight effects or effects on clinical |
| | (100% NR1), IC | group | | | chemistry, hematology or bone marrow |
| | Group 3: 2e6 NR1 | | | | No NR1 drug product-rformation of |
| | (100% NR1), IC | | | | t [symbol] in brain or any peripheral tissue |
| | Group 4: 2e6 total dose (50% | | | | Animal [symbol] shown to be supportive of the |
| | NR1:50% H9), IC | | | | development of ter [symbol] by including NR1 |
| | Group 5: 2e6 total dose (75% | | | | treatment groups with added H9 [symbol] |
| | NR1:25% H9), IC | | | | H9 spike level dose-dependent occurence of |
| | Group 6: 2e6 total dose (90% | | | | ter [symbol] or [symbol] ranging from [symbol] 7% in |
| | NR1:10% H9), IC | | | | Group 4 to 5.3% in Group [symbol] |
| | Group 7: 2e6 total dose (99% | | | | Ter [symbol] at hiogh levels of added H9 cells |
| | NR1:1% H9), IC | | | | detectable macro- an dmicroscopically and |
| | Group 8: 2e6 total dose | | | | consisted of tissues derived from endoderm, |
| | (99.9% NR1:0.1% H9), IC | | | | mesoderm and ectoderm germ lines |
| | Group 9: 1e6 H9 (100%), IC | | | | Tumors at lower levels (4/20 animals at 1% |
| | Group 10: 2e3 H9 (100%), IC | | | | and 1/19 animals at 0.1% H9 spike) only |
| | Group 11: 1e7 NR1 | | | | detected microscopically and did not incluse |
| | (100% NR1), IM | | | | all three germ lines |
| | Group 12: 1e7 total dose | | | | No cell engraftment or ter [symbol] in H9-only |
| | (50% NR1:50% H9), IM | | | | groups injected IC into the brain [symbol] IM |
| | Group 13: 1e7 total dose | | | | No ter [symbol] were detected in animals that |
| | (90.9% NR1:0.1% H9), IM | | | | received NR1 drug product only at dose up to |
| | | | | | 2e6 per animal and were followed up to 180 [symbol] |
| | | | | | IHC straining for human nuclear protein HuNu |
| | | | | | showed persistence of NR1 cells to 180 days |
| | | | | | post-transplant in NR1-only groups. |
| | | | | | [symbol] PCR analysis detected NR1 cells at the |
| | | | | | injection site at [symbol] 28 days (16 and 2 [symbol] |
| | | | | | copies of NR1 DNA/ [symbol] brain DNA at low |
| | | | | | and high dose) and 180 days (38 amd 50 |
| | | | | | copies at lowe and high dose) post-injection |
| | | | | | No evidence of NR1 cells or [symbol] formation |
| | | | | | distant from the brain injection site |
| Adult athymic nude rats | Part 1: | 5-6 | 1 to 2 × 10⁶ | Not | All animals survived to necropsy |
| (C [symbol] NIH-F [symbol] ) | Meduim: Plasmalyte-A-HSA | females | NR1 cells | determined | No macroscopic findings or adverse effects at |
| Neonatal athymic nude rats | with or without 40% | per | | | 1 × 10⁶ or 2 × 10⁶ NR1 up to 30 days post- |
| (C [symbol] NIH-F [symbol] ) | Matrigel, IC | group | | | transplant |
| Adult mice, NSG | Group 1: Adult athymic nude | | | | No evidence of tumor or ectopic tissue |
| (NOD [symbol] ) | rats; in Plasmalyte-A | | | | formation |
| | HSA + 40% Matrigel; 2e6 | | | | Level of NR1 cell [symbol] was greatest with |
| | NR1, IC | | | | Matrigel in adult nude rats |
| | Group 2: Neonatal athymic | | | | |
| | nude rats; in Plasmalyte-A- | | | | |
| | HSA + 40% Matrigel; 1e6 | | | | |
| | NR1, IC | | | | |
| | Group 3: NSG mice; in | | | | |
| | Plasmalyte-A-HSA + 40% | | | | |
| | Matrigel; 1e6 NR1, IC | | | | |
| | Part 2: | | | | |
| | Group 4: Adult athymic nude | | | | |
| | rin Plasmalyte-A- | | | | |
| | HSA + 40% Matrigel; 2e6 | | | | |
| | NR1, IC | | | | |
| | Group 5: Adult athymic nude | | | | |
| | rats; Plasmalyte-A-HSA + 40% | | | | |
| | Matrigel; 2e6 NR1, IC | | | | |
| Adult athymic nude rats | Group 1: 2e6 NR1 in | 3-20 | 2 × 10⁶ | Not | All animals survived to scheduled necropsy |
| (C [symbol] NIH-F [symbol] ) | Plasmalyte-A-HSA, IC | females | NR1 cells | determined | No macro- or microscopic signs of adverse |
| | Group 2: 2e6 NR1 in | per | | | effects at 2 × 10⁶ NR1 up to 60 days post- |
| | Plasmalyte-A-HSA + 40% | group | | | transplant |
| | Matrigel, IC | | | | No tumor or ectopic tissue fromation |
| | Group 3: Plasmalyte-A-HSA | | | | Cell survival highest in the presence of |
| | control, IC | | | | Matrigel |
| | Group 4: Plasmalyte-A-HSA + | | | | |
| | 40% Matrigel control, IC | | | | |
| | Group 5: 2e6 NR1 in | | | | |
| | Plasmalyte-A-HSA, IC | | | | |
| | Group 6: 2e6 NR1 in | | | | |
| | Plasmalyte-A-HSA + 40% | | | | |
| | Matrigel, IC | | | | |
| Adult athymic nude rats | Groups 1-6: NR1 in | 24 | 1 × 10⁶ | Not | No adverse events or tumors or ectopic tissue |
| (C [symbol] NIH-F [symbol] ) | DMEM/F12; 1e6, IC | males | NR1 cells | determined | formation for up to 6 months at a dose of 1 × 10⁶ |
| | Group 7: H9 in DMEM/F12; | per | | | NR1 |
| | 1e6, IC | group | | | All animals treated with 1 × 10⁶ H9 develpoed |
| | Groups 8-13: NR1 in | | | | brain tumors by 7 weeks |
| | DMEM/F12 + 30% Matrigel; | | | | Matrigel enhanced cell survival |
| | 1e6, IC | | | | |
| | Group 14: H9 in DMEM/F12 + | | | | |
| | 30 % Matrigel; 1e6, IC | | | | |

IC: Intracerebral via intrapa [symbol] injection into the cortex and [symbol] of both hemispheres of the brain
IM: Intermuscular into the biceps f [symbol] of the left [symbol] [symbol]
indicates data missing or illegible when filed

### Example 13

### Potency Assay Studies

Preclinical studies have demonstrated that NR1 cells secrete a spectrum of proteins with functions that could contribute to the repair of stroke-damaged brain tissue. We have chosen six of these proteins as candidate Potency Assay markers, which are measures of NR1 biological activity, based on their known roles in axonal sprouting, neurogenesis, synaptogenesis and matrix/tissue remodeling.

For each candidate protein listed below, commercially available kits that meet FDA requirements were identified (three ELISA kits and three ProCartaPlex®/Luminex kits).

TABLE 12

| | |
|---|---|
| Galectin/LGALS1 | One of the highest expressed NR1 secreted factors both in vivo and in |
| | vitro. Known to polarize macrophages to the M2 state. M2 macrophages |
| | produce anti-inflammatory mediators and are implicated in |
| | immunomodulation and tissue repair responses. |
| SDF1(a) CXCL12 | An angiogenic factor. Also a chemotactic factor for lymphocytes and |
| | neural progenitors. |
| VEGF | An angiogenic factor. Promotes axonal sprouting neurogenesis, |
| | synaptogenesis. |
| CSF3/G-CSF | Role in neural plasticity, inflammation and angiogenesis. |
| SPARC | Involved in cell proliferation, repair of tissue damage, and morphogenic |
| | processes such as remodeling of the extra-cellular matrix. |
| PAI1/serpine1 | Promotes extracellular matrix remodeling. |

Acceptance of each kit as a component of the Potency Assay depends on the kit meeting the following criteria:
Objective-1: Select several proteins secreted by NR1 cells as candidates for a Potency Assay. The criteria used to select these candidates are based on preclinical studies.
Objective-2: Identify commercially available kits that meet FDA requirements of monoclonal antibodies for both the capture and detector antibody.
Objective-3: Determine if the performance of the available kits is not altered in the presence of Complete Media (CM) Determine the Performance the Standard Titer of the assay kits in the presence of NR1 Complete Media.
Objective-4: Confirm the specificity of each portion of the Potency Assay.
Objective-5: Determine if a limited number of growth factors are either detected or cause any interference of the target antigen.
Objective-6: Determine if an on-target candidate is still accurately quantified with varying concentration of an off target candidate, each portion of the Potency Assay will be tested with a) an off-target candidate as a negative control and b) a series of samples where the on-target candidate is diluted with a varying concentration of the off-target candidate.
Objective-7: Determine the appropriate dilutions for each candidate's qualification using NR1 conditions media (CM) generated under various growth conditions. Once that is established, each component of the assay will be tested for intra/inter-assay precision, accuracy by linear dilution, dynamic range and reproducibility.

The three ELISA kits and three of the four ProCartaPlex®/Luminex kits have met Objectives 1-6. We are in the process of completing Objective 7 for all kits. We expect completion of the remaining objectives and assay qualification in time for the submission of NR1 stem cell therapy for any phase Ill clinical trial and subsequent licensing/marketing application.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method for treating a subject suffering a stroke or traumatic brain injury by increasing exogenous trophic factors in the cerebral cortex to augment endogenous neural repair processes in the subject, the method comprising:
administering to the subject a therapeutically effective amount of non-genetically modified NR1 ES-derived neural stem cells having a normal karyotype, which cells express trophic factors, so as to restore neurologic function in the subject, thereby treating the subject suffering a stroke or traumatic brain injury.

2. The method of claim 1, wherein the neural stem cells are isolated non-genetically modified NR1 ES-derived neural stem cells.

3. The method of claim 1, wherein the stroke is an ischemic stroke or hemorrhagic stroke.

4. The method of claim 1, wherein the administration is via cortical or subcortical brain transplantation.

5. The method of claim 1, wherein administration of the NR1 ES-derived neural stem cells comprises implantation of the NR1 ES-derived neural stem cells into or near the cerebral cortex of the subject.

6. The method of claim 5, wherein the cerebral cortex of the subject includes any of the prefrontal cortex, motor association cortex, primary motor cortex or primary somatosensory cortex.

7. The method of claim 1, wherein administration comprises transplantation of NR1 ES-derived neural stem cells into a subcortical area or cortical area of the brain of the subject at least one week after the stroke or traumatic brain injury.

8. The method of claim 7, wherein the subcortical area of the brain is any of the hippocampus, amygdala, extended amygdala, claustrum, basal ganglia, or basal forebrain.

9. The method of claim 7, wherein the cortical areas of the brain is any of the prefrontal cortex, motor association cortex, primary motor cortex or primary somatosensory cortex.

10. The method of claim 1, wherein the NR1 ES-derived neural stem cells express one or more of CoIA1, LGALS1, TGF-B3, TIMP1, COL6A1, COL3A1, MMP2, SPARC, NRG3, SDF1(a), Galectin 1, FGF18, CSF3, CCL2 (aka MCP-1), FGF7, FGF17, PAI1/serpine 1, VEGF-A, MCP1 (CCL2), and/or SDF1α or a combination thereof.

11. The method of claim 1, wherein the trophic factors are factors that can augment the repair processes in neural neovascularization by increasing blood flow and vascular signals within the cortical and/or subcortical brain area and increasing structural plasticity.

12. The method of claim 11, wherein the trophic factors comprise a combination of a growth factor, cytokine and/or extracellular matrix protein.

13. The method of claim 11, wherein the extracellular matrix protein is any of a CoIA1, LGALS1, TGF-B3, TIMP1, COL6A1, COL3A1, MMP2, SPARC, NRG3, SDF1(a), Galectin 1, FGF18, CSF3, CCL2 (aka MCP-1), FGF7, FGF17, PAI1/serpine 1, VEGF-A, MCP1 (CCL2), and/or SDF1a or a combination thereof.

14. The method of claim 11, wherein increased structural plasticity involves any of synaptogenesis, dendritic branching and axonal sprouting.

15. The method of claim 1, wherein the restored neurologic function partially or fully restores an upper extremity function.

16. The method of claim 15, wherein the upper extremity function is raising or lifting an arm of the subject.

17. The method of claim 1, wherein restoring neurologic function includes restoring the ability to walk or balance or reversing a gait impairment, inability to walk, or loss of balance.

18. The method of claim 17, wherein gait impairment comprises any of decreased walking velocity, asymmetric walking pattern, decreased stride length, increased stride width, prolonged swing phase of affected limb, diminished ability to negotiate physical obstacle, diminished ability to adjust walking to changes in terrain, loss of rhythmic movement, diminished ability to move across a beam and a combination thereof.

19. The method of claim 17, wherein reversing the impaired mobility in the subject comprises any of greater walking velocity, an increase in symmetric walking pattern, greater stride length, decreased stride width, reduced duration of swing phase of affected limb, greater ability to negotiate physical obstacle, greater ability to adjust walking to changes in terrain, increased rhythmic movement, greater ability to move across a beam or ladder and a combination thereof.

20. The method of claim 1, wherein the brain lesion is located outside the cortex or subcortical brain region of the subject.

21-41. (canceled)
